(19) United States
(12) Patent Application Publication
(10) Pub. No.: US 2018/0185309 A1
(43) Pub. Date: Jul. 5, 2018

(54) METHODS AND COMPOSITIONS FOR TREATING NEPHROPATHY

(71) Applicants: The Regents of the University of California, Oakland, CA (US); United States of America, as Represented by Secretary of Health and Human Services, Rockville, MD (US)

(72) Inventors: Bruce D. HAMMOCK, Davis, CA (US); Fawaz G. HAJ, Davis, CA (US); Ahmed BETTAIEB, Knoxville, TN (US); Darryl C. Zeldin, Chapel Hill, NC (US)

(73) Assignees: The Regents of the University of California, Oakland, CA (US); United States of America, as Represented by Secretary of Health and Human Services, Rockville, MD (US)

(21) Appl. No.: 15/739,718

(22) PCT Filed: Jun. 2, 2016
(86) PCT No.: PCT/US16/35548

§ 371 Date: Dec. 23, 2017

Related U.S. Application Data
(60) Provisional application No. 62/188,544, filed on Jul. 3, 2015.

(51) Int. Cl. A61K 31/192; A61K 31/20; A61K 31/7105; A61K 31/17; A61K 31/415; A61K 31/4468; A61K 45/06; A61P 13/12

(52) U.S. Cl. CPC A61K 31/192; A61K 31/20; A61K 31/7105; A61P 13/12; A61K 31/415; A61K 31/4468; A61K 45/06; A61K 31/17

(57) ABSTRACT

Provided are compositions and methods for improving podocyte and kidney function and glucose homeostasis in diabetic and pre-diabetic states.

### CROSS-REFERENCE TO RELATED APPLICATIONS

This application is the U.S. national phase under 35 U.S.C. § 371 of Intl. Appl. No. PCT/US2016/035548, filed on Jun. 2, 2016, which claims the benefit under 35 U.S.C. § 119(e) of U.S. Provisional Application No. 62/188,544, filed on Jul. 3, 2015, which are hereby incorporated herein by reference in their entireties for all purposes.

### STATEMENT OF GOVERNMENTAL SUPPORT

This invention was made with government support under Grant numbers R01DK090492, R01DK095359 and K99DK100736, awarded by the National Institutes of Health. The government has certain rights in the invention.

### FIELD

Provided are compositions and methods for improving podocyte and kidney function and glucose homeostasis in diabetic and pre-diabetic states.

### BACKGROUND

The increasing prevalence of diabetes mellitus has become a global health issue and it is projected that the number of people with diabetes worldwide will increase from 382 million in 2013 to 592 by 2035 [1]. Diabetic nephropathy (DN) is one of the most devastating complications of diabetes and the leading cause of end stage kidney disease [2]. DN begins with proteinuria then progresses to renal inflammation and decline in glomerular filtration barrier (GFB) [3, 4]. GFB is composed of two cell types, podocytes and glomerular endothelial cells [5]. The podocyte is particularly important in maintaining the integrity of GFB in humans [6, 7], and there is growing evidence that podocyte dysfunction plays an important role in the pathogenesis of DN [8]. Elucidating the mechanisms underlying podocyte function is critical for understanding disease pathogenesis and developing better therapies.

Arachidonic acids are metabolized by cyclooxygenases (COX), lipoxygenases (LOX), and cytochrome P450's (CYP) to eicosanoids which are key regulators of numerous biological processes. CYP epoxygenase enzymes (including CYP2C, 2J) metabolize arachidonic acid to biologically active epoxyeicosatrienoic acids (EETs) [9] which are anti-hypertensive, anti-inflammatory, and anti-allodynic [10-12]. However, EETs are rapidly hydrolyzed by soluble epoxide hydrolase (sEH, encoded by Ephx2) into the less biologically active metabolites, dihydroxyeicostrienoic acids (DHETs) [9, 13-15]. sEH is a conserved cytosolic enzyme that is widely distributed and highly expressed in the kidney, liver and vasculature [14]. A growing body of evidence implicates sEH in kidney function. Pharmacological inhibition of sEH reduces renal injury and inflammation in salt-sensitive hypertension and in hypertensive type 2 diabetes rats [16-18]. In addition, sEH inhibition prevents renal interstitial fibrosis in unilateral ureteral obstruction mouse model [19, 20]. Moreover, Ephx2 whole-body knockout (KO) mice display reduced renal inflammation in DOCA-salt hypertension model [21] and reduced renal injury [22]. While these studies implicate sEH in kidney function they utilize systemic deletion and inhibition approaches. Tissue- and cell-specific contribution of sEH to kidney function and systemic homeostasis remain to be elucidated.

### SUMMARY

In one aspect, provided are methods for improving, increasing and/or promoting podocyte and/or kidney function and/or mitigating, reducing, inhibiting and/or delaying podocyte and/or kidney degradation and/or failure in a subject in need thereof. In varying embodiments, the methods comprise administering to the subject an inhibitor of endoplasmic reticulum (ER) stress. In varying embodiments, the methods comprise administering to the subject an agent that increases the production and/or level of epoxygenated fatty acids. In varying embodiments, the methods comprise co-administering to the subject an agent that increases the production and/or level of epoxygenated fatty acids and an inhibitor of endoplasmic reticular (ER) stress. In varying embodiments, the agent that increases the production and/or level of epoxygenated fatty acids and the inhibitor of endoplasmic reticular stress are administered at a subtherapeutic dose. In varying embodiments, one or both of the agent that increases the production and/or level of epoxygenated fatty acids and the inhibitor of endoplasmic reticular stress are targeted to the kidneys. In varying embodiments, the agent that increases the production and/or level of epoxygenated fatty acids and the inhibitor of endoplasmic reticular stress are concurrently co-administered. In varying embodiments, the agent that increases the production and/or level of epoxygenated fatty acids and the inhibitor of endoplasmic reticular stress are sequentially co-administered. In varying embodiments, the inhibitor of ER stress acts as a molecular chaperone that facilitates correct protein folding and/or prevents protein aggregation and/or acts to enhance autophagy. In varying embodiments, the inhibitor of ER stress modifies protein folding, regulates glucose homeostasis and/or reduces lipid overload. In varying embodiments, the inhibitor of endoplasmic reticular stress performs one or more of the following: a) prevents, reduces and/or inhibits phosphorylation of PERK (Thr980), Ire1α (Ser727), eIF2α (Ser51), p38 and/or JNK1/2; b) prevents, reduces and/or inhibits cleavage of ATF6 and/or XBP1; and/or c) prevents, reduces and/or inhibits mRNA expression of BiP, ATF4 and/or XBP1. In varying embodiments, the inhibitor of endoplasmic reticular stress is selected from the group consisting of 4-phenyl butyric acid (“PBA”), 3-phenylpropionic acid (3-PPA), 5-phenylvaleric acid (5-PVA), 6-phenylhexanoic acid (6-PHA), butyrate, tauroursodeoxycholic acid, trehalose, deuterated water, docosahexaenoic acid (“DHA”), eicosapentaenoic acid (“EPA”), vitamin C, arabitol, mannose, glycerol, betaine, sarcosine, trimethylamine-N oxide, DMSO and mixtures thereof. In varying embodiments, the inhibitor of endoplasmic reticular stress is selected from the group consisting of 4-phenyl butyric acid (4-PBA), 3-phenylpropionic acid (3-PPA), 5-phenylvaleric acid (5-PVA), 6-phenylhexanoic acid (6-PHA), esters thereof, pharmaceutically acceptable salts thereof, and mixtures thereof. In varying embodiments, the agent that increases the production and/or level of epoxygenated fatty acids comprises one or more epoxygenated fatty acids. In varying embodiments, the epoxygenated fatty acids are selected from the group consisting of cis-epoxyeicosantrienoic acids (“EETs”), epoxides of linoleic acid, epoxides of eicosapentaenoic acid (“EPA”), epoxides of docosahexaenoic acid (“DHA”), epoxides of the arachidonic acid (“AA”), epoxides of cis-7,10,13,16,19-docosapentaenoic acid, and mixtures thereof. In varying embodiments, the agent that increases the production and/or level of epoxygenated fatty acids increases the production and/or levels of cis-epoxyeicosantrienoic acids (“EETs”). In varying embodiments, the agent that increases the production and/or level of EETs is an inhibitor of soluble epoxide hydrolase (“sEH”). In varying embodiments, the inhibitor of sEH comprises an inhibitory nucleic acid that specifically targets soluble epoxide hydrolase (“sEH”). In varying embodiments, the inhibitory nucleic acid is targeted to kidney tissue. In varying embodiments, the inhibitory nucleic acid is targeted to podocyte cells. In varying embodiments, the inhibitory nucleic acid is selected from the group consisting of short interfering RNA (siRNA), short hairpin RNA (shRNA), small temporal RNA (stRNA), and micro-RNA (miRNA). In varying embodiments, the inhibitor of sEH comprises a primary pharmacophore selected from the group consisting of a urea, a carbamate, and an amide. In varying embodiments, the inhibitor of sEH comprises a cyclohexyl moiety, aromatic moiety, substituted aromatic moiety or alkyl moiety attached to the pharmacophore. In varying embodiments, the inhibitor of sEH comprises a cyclohexyl ether moiety attached to the pharmacophore. In varying embodiments, the inhibitor of sEH comprises a phenyl ether or piperidine moiety attached to the pharmacophore. In varying embodiments, the inhibitor of sEH comprises a polyether secondary pharmacophore. In varying embodiments, the inhibitor of sEH has an IC50 of less than about 100 μM. In varying embodiments, the inhibitor of sEH is selected from the group consisting of:
a) 3-(4-chlorophenyl)-1-(3,4-dichlorphenyl)urea or 3,4,4′-trichlorocarbanilide (TCC; compound 295);
b) 12-(3-adamantan-1-yl-ureido) dodecanoic acid (AUDA; compound 700);
c) 1-adamantanyl-3-{5-[2-(2-ethoxyethoxy)ethoxy]pentyl]}urea (AEPU; compound 950);
d) 1-(1-acetypiperidin-4-yl)-3-adamantanylurea (APAU; compound 1153);
e) trans-4-[4-(3-Adamantan-1-yl-ureido)-cyclohexyloxy]-benzoic acid (tAUCB; compound 1471);
f) cis-4-[4-(3-Adamantan-1-yl-ureido)-cyclohexyloxy]-benzoic acid (cAUCB; compound 1686);
g) 1-(1-methylsulfonyl-piperidin-4-yl)-3-(4-trifluoromethoxy-phenyl)-urea (TUPS; compound 1709);
h) trans-4-{4-[3-(4-Trifluoromethoxy-phenyl)-ureido]-cyclohexyloxy}-benzoic acid (tTUCB; compound 1728);
i) 1-trifluoromethoxyphenyl-3-(1-propionylpiperidin-4-yl) urea (TPPU; compound 1770);
j) 1-(1-ethylsulfonyl-piperidin-4-yl)-3-(4-trifluoromethoxy-phenyl)-urea (TUPSE; compound 2213);
k) 1-(1-(cyclopropanecarbonyl)piperidin-4-yl)-3-(4-(trifluoromethoxy)phenyl)urea (CPTU; compound 2214);
l) trans-N-methyl-4-[4-(3-Adamantan-1-yl-ureido)-cyclohexyloxy]-benzamide (tMAUCB; compound 2225);
m) trans-N-methyl-4-[4-((3-trifluoromethyl-4-chlorophenyl)-ureido)-cyclohexyloxy]-benzamide (tMTCUCB; compound 2226);
n) cis-N-methyl-4-{4-[3-(4-trifluoromethoxy-phenyl)-ureido]-cyclohexyloxy}-benzamide (cMTUCB; compound 2228);
o) 1-cycloheptyl-3-(3-(1,5-diphenyl-1H-pyrazol-3-yl)propyl)urea (HDP₃U; compound 2247);
p) trans-2-(4-(4-(3-(4-trifluoromethoxy-phenyl)-ureido)-cyclohexyloxy)-benzamido)-acetic acid (compound 2283);
q) N-(methylsulfonyl)-4-(trans-4-(3-(4-trifluoromethoxy-phenyl)-ureido)-cyclohexyloxy)-benzamide (compound 2728);
r) 1-(trans-4-(4-(1H-tetrazol-5-yl)-phenoxy)-cyclohexyl)-3-(4-(trifluoromethoxy)-phenyl)-urea (compound 2806);
s) 4-(trans-4-(3-(2-fluorophenyl)-ureido)-cyclohexyloxy)-benzoic acid (compound 2736);
t) 4-(4-(3-(4-(trifluoromethoxy)-phenyl)-ureido)-phenoxy)-benzoic acid (compound 2803);
u) 4-(3-fluoro-4-(3-(4-(trifluoromethoxy)-phenyl)-ureido)-phenoxy)-benzoic acid (compound 2807);
v) N-hydroxy-4-(trans-4-(3-(4-(trifluoromethoxy)-phenyl)-ureido)-cyclohexyloxy)-benzamide (compound 2761);
w) (5-methyl-2-oxo-1,3-dioxol-4-yl)methyl 4-((1r,4r)-4-(3-(4-(trifluoromethoxy)-phenyl)-ureido)-cyclohexyloxy)-benzoate (compound 2796);
x) 1-(4-oxocyclohexyl)-3-(4-(trifluoromethoxy)-phenyl)-urea (compound 2809);
y) methyl 4-(4-(3-(4-(trifluoromethoxy)-phenyl)-ureido)-cyclohexylamino)-benzoate (compound 2804);
z) 1-(4-(pyrimidin-2-yloxy)-cyclohexyl)-3-(4-(trifluoromethoxy)-phenyl)-urea (compound 2810); and
aa) 4-(trans-4-(3-(4-(difluoromethoxy)-phenyl)-ureido)-cyclohexyloxy)-benzoic acid (compound 2805). In varying embodiments, the inhibitor of sEH is co-administered at a subtherapeutic dose. In varying embodiments, the subject is a human. In varying embodiments, the subject has or is suspected of having diabetes. In varying embodiments, the subject has or is suspected of having pre-diabetes. In varying embodiments, the subject is exhibiting one or more symptoms of renal function deficiency. In varying embodiments, the subject is exhibiting one or more symptoms selected from the group consisting of proteinuria, renal inflammation and decline in glomerular filtration barrier (GFB). In varying embodiments, the methods further comprise co-administering an inhibitor of sodium-glucose cotransporter-2 (SGLT2). In varying embodiments, the inhibitor of SGLT2 is selected from the group consisting of canagliflozin, dapagliflozin, empagliflozin, metformin, linagliptin, and mixtures thereof.

In another aspect, provided are kits for use in improving, increasing and/or promoting podocyte and/or kidney function and/or mitigating, reducing, inhibiting and/or delaying podocyte and/or kidney degradation and/or failure in a subject in need thereof, the kit comprising an agent that increases the production and/or level of epoxygenated fatty acids and an inhibitor of endoplasmic reticular stress. In varying embodiments, the inhibitor of endoplasmic reticular stress is selected from the group consisting of 4-phenyl butyric acid (“PBA”), 3-phenylpropionic acid (3-PPA), 5-phenylvaleric acid (5-PVA), 6-phenylhexanoic acid (6-PHA), butyrate, tauroursodeoxycholic acid, trehalose, deuterated water, docosahexaenoic acid (“DHA”), eicosapentaenoic acid (“EPA”), vitamin C, arabitol, mannose, glycerol, betaine, sarcosine, trimethylamine-N oxide, DMSO and mixtures thereof. In varying embodiments, the inhibitor of endoplasmic reticular stress is selected from the group consisting of 4-phenyl butyric acid (4-PBA), 3-phenylpropionic acid (3-PPA), 5-phenylvaleric acid (5-PVA), 6-phenylhexanoic acid (6-PHA), esters thereof, pharmaceutically acceptable salts thereof and mixtures thereof. In varying embodiments, the agent that increases the production and/or level of EETs is an inhibitory nucleic acid that specifically targets soluble epoxide hydrolase (“sEH”). In varying embodiments, the agent that increases the production and/or level of EETs is an inhibitor of soluble epoxide hydrolase (“sEH”). In varying embodiments, the inhibitor of sEH comprises a primary pharmacophore selected from the group consisting of a urea, a carbamate, and an amide. In varying embodiments, the inhibitor of sEH comprises a cyclohexyl moiety, aromatic moiety, substituted aromatic moiety or alkyl moiety attached to the pharmacophore. In varying embodiments, the inhibitor of sEH comprises a cyclohexyl ether moiety attached to the pharmacophore. In varying embodiments, the inhibitor of sEH comprises a phenyl ether or piperidine moiety attached to the pharmacophore. In varying embodiments, the inhibitor of sEH comprises a polyether secondary pharmacophore. In varying embodiments, the inhibitor of sEH has an IC50 of less than about 100 μM. Further embodiments of the inhibitor of sEH are as described above and herein. In varying embodiments, the kits further comprise an inhibitor of sodium-glucose cotransporter-2 (SGLT2). In varying embodiments, the inhibitor of SGLT2 is selected from the group consisting of canagliflozin, dapagliflozin, empagliflozin, metformin, linagliptin, and mixtures thereof. In varying embodiments, the agent that increases the production and/or level of epoxygenated fatty acids and the inhibitor of endoplasmic reticular stress are provided in a mixture. In varying embodiments, the agent that increases the production and/or level of epoxygenated fatty acids and the inhibitor of endoplasmic reticular stress are provided in separate containers.

In a further aspect, provided are compositions comprising an agent that increases the production and/or level of epoxygenated fatty acids and an inhibitor of endoplasmic reticular (ER) stress. In varying embodiments, the inhibitor of endoplasmic reticular stress is selected from the group consisting of 4-phenyl butyric acid (“PBA”), 3-phenylpropionic acid (3-PPA), 5-phenylvaleric acid (5-PVA), 6-phenylhexanoic acid (6-PHA), butyrate, tauroursodeoxycholic acid, trehalose, deuterated water, docosahexaenoic acid (“DHA”), eicosapentaenoic acid (“EPA”), vitamin C, arabitol, mannose, glycerol, betaine, sarcosine, trimethylamine-N oxide, DMSO and mixtures thereof. In varying embodiments, the inhibitor of endoplasmic reticular stress is selected from the group consisting of 4-phenyl butyric acid (4-PBA), 3-phenylpropionic acid (3-PPA), 5-phenylvaleric acid (5-PVA), 6-phenylhexanoic acid (6-PHA), esters thereof, pharmaceutically acceptable salts thereof and mixtures thereof. In varying embodiments, the agent that increases the production and/or level of EETs is an inhibitory nucleic acid that specifically targets soluble epoxide hydrolase (“sEH”). In varying embodiments, the agent that increases the production and/or level of EETs is an inhibitor of soluble epoxide hydrolase (“sEH”). In varying embodiments, the inhibitor of sEH comprises a primary pharmacophore selected from the group consisting of a urea, a carbamate, and an amide. In varying embodiments, the inhibitor of sEH comprises a cyclohexyl moiety, aromatic moiety, substituted aromatic moiety or alkyl moiety attached to the pharmacophore. In varying embodiments, the inhibitor of sEH comprises a cyclohexyl ether moiety attached to the pharmacophore. In varying embodiments, the inhibitor of sEH comprises a phenyl ether or piperidine moiety attached to the pharmacophore. In varying embodiments, the inhibitor of sEH comprises a polyether secondary pharmacophore. In varying embodiments, the inhibitor of sEH has an IC50 of less than about 100 μM. Further embodiments of the inhibitor of sEH are as described above and herein.

### Definitions

Units, prefixes, and symbols are denoted in their Systeme International de Unites (SI) accepted form. Numeric ranges are inclusive of the numbers defining the range. Unless otherwise indicated, nucleic acids are written left to right in 5′ to 3′ orientation; amino acid sequences are written left to right in amino to carboxy orientation. The headings provided herein are not limitations of the various aspects or embodiments, which can be had by reference to the specification as a whole. Accordingly, the terms defined immediately below are more fully defined by reference to the specification in its entirety. Terms not defined herein have their ordinary meaning as understood by a person of skill in the art.

The terms “podocytes” and “visceral epithelial cells” interchangeably refer to cells in the Bowman's capsule in the nephron of the kidneys that wrap around the capillaries of the glomerulus. The Bowman's capsule filters blood, holding back large molecules such as proteins, and passing through small molecules such as water, salts, and sugar, as the first step in forming urine. See, Dorland's Medical Dictionary, 32^{nd} edition, 2011, Saunders.

The phrase “endoplasmic reticulum (ER) stress” refers to disruption of processes performed by the endoplasmic reticulum, including the synthesis, modification, folding and delivery of proteins to their proper target sites within the secretory pathway and the extracellular space. ER stress can be caused by, e.g., disruption of protein folding, aberrations in lipid metabolism, or disruption of cell wall biogenesis. See, e.g., Schröder and Kaufman, Mutation Research (2005) 569:29-63.

“cis-Epoxyeicosatrienoic acids” (“EETs”) are biomediators synthesized by cytochrome P450 epoxygenases. As discussed further in a separate section below, while the use of unmodified EETs is the most preferred, derivatives of EETs, such as amides and esters (both natural and synthetic), EETs analogs, and EETs optical isomers can all be used in the methods, both in pure form and as mixtures of these forms. For convenience of reference, the term “EETs” as used herein refers to all of these forms unless otherwise required by context.

“Epoxide hydrolases” (“EH;” EC 3.3.2.3) are enzymes in the alpha beta hydrolase fold family that add water to 3-membered cyclic ethers termed epoxides.

“Soluble epoxide hydrolase” (“sEH”) is an epoxide hydrolase which in endothelial and smooth muscle cells converts EETs to dihydroxy derivatives called dihydroxyeicosatrienoic acids (“DHETs”). The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1):197-201 (1993). The amino acid sequence of human sEH is SEQ ID NO.:1, while the nucleic acid sequence encoding the human sEH is SEQ ID NO.:2. (The sequence set forth as SEQ ID NO.:2 is the coding portion of the sequence set forth in the Beetham et al. 1993 paper and in the NCBI Entrez Nucleotide Browser at accession number L05779, which include the 5′ untranslated region and the 3′ untranslated region.) The evolution and nomenclature of the gene is discussed in Beetham et al., DNA Cell Biol. 14(1):61-71 (1995). Soluble epoxide hydrolase represents a single highly conserved gene product with over 90% homology between rodent and human (Arand et al., FEBS Lett., 338:251-256 (1994)). Unless otherwise specified, as used herein, the terms “soluble epoxide hydrolase” and “sEH” refer to human sEH.

Unless otherwise specified, as used herein, the term “sEH inhibitor” (also abbreviated as “sEHI”) refers to an inhibitor of human sEH. Preferably, the inhibitor does not also inhibit the activity of microsomal epoxide hydrolase by more than 25% at concentrations at which the inhibitor inhibits sEH by at least 50%, and more preferably does not inhibit mEH by more than 10% at that concentration. For convenience of reference, unless otherwise required by context, the term “sEH inhibitor” as used herein encompasses prodrugs which are metabolized to active inhibitors of sEH. Further for convenience of reference, and except as otherwise required by context, reference herein to a compound as an inhibitor of sEH includes reference to derivatives of that compound (such as an ester of that compound) that retain activity as an sEH inhibitor.

Cytochrome P450 (“CYP450”) metabolism produces cis-epoxydocosapentaenoic acids (“EpDPEs”) and cis-epoxyeicosatetraenoic acids (“EpETEs”) from docosahexaenoic acid (“DHA”) and eicosapentaenoic acid (“EPA”), respectively. These epoxides are known endothelium-derived hyperpolarizing factors (“EDHFs”). These EDHFs, and others yet unidentified, are mediators released from vascular endothelial cells in response to acetylcholine and bradykinin, and are distinct from the NOS- (nitric oxide) and COX-derived (prostacyclin) vasodilators. Overall cytochrome P450 (CYP450) metabolism of polyunsaturated fatty acids produces epoxides, such as EETs, which are prime candidates for the active mediator(s). 14(15)-EpETE, for example, is derived via epoxidation of the 14,15-double bond of EPA and is the ω-3 homolog of 14(15)-EpETrE (“14(15)EET”) derived via epoxidation of the 14,15-double bond of arachidonic acid.

“IC₅₀” refers to the concentration of an agent required to inhibit enzyme activity by 50%.

The term “neuroactive steroid” or “neurosteroids” interchangeably refer to steroids that rapidly alter neuronal excitability through interaction with neurotransmitter-gated ion channels, and which may also exert effects on gene expression via intracellular steroid hormone receptors. Neurosteroids have a wide range of applications from sedation to treatment of epilepsy and traumatic brain injury. Neurosteroids can act as allosteric modulators of neurotransmitter receptors, such as GABA_{A}, NMDA, and sigma receptors. Progesterone (PROG) is also a neurosteroid which activates progesterone receptors expressed in peripheral and central glial cells. Several synthetic neurosteroids have been used as sedatives for the purpose of general anaesthesia for carrying out surgical procedures. Exemplary sedating neurosteroids include without limitation alphaxolone, alphadolone, hydroxydione and minaxolone.

By “physiological conditions” is meant an extracellular milieu having conditions (e.g., temperature, pH, and osmolarity) which allows for the sustenance or growth of a cell of interest.

“Micro-RNA” (“miRNA”) refers to small, noncoding RNAs of 18-25 nt in length that negatively regulate their complementary mRNAs at the posttranscriptional level in many eukaryotic organisms. See, e.g., Kurihara and Watanabe, Proc Natl Acad Sci USA 101(34):12753-12758 (2004). Micro-RNA's were first discovered in the roundworm C. elegans in the early 1990s and are now known in many species, including humans. As used herein, it refers to exogenously administered miRNA unless specifically noted or otherwise required by context.

The term “therapeutically effective amount” refers to that amount of the compound being administered sufficient to prevent or decrease the development of one or more of the symptoms of the disease, condition or disorder being treated (e.g., fibrosis and/or inflammation).

The terms “prophylactically effective amount” and “amount that is effective to prevent” refer to that amount of drug that will prevent or reduce the risk of occurrence of the biological or medical event that is sought to be prevented. In many instances, the prophylactically effective amount is the same as the therapeutically effective amount.

“Subtherapeutic dose” refers to a dose of a pharmacologically active agent(s), either as an administered dose of pharmacologically active agent, or actual level of pharmacologically active agent in a subject that functionally is insufficient to elicit the intended pharmacological effect in itself (e.g., to obtain analgesic, anti-inflammatory, and/or anti-fibrotic effects), or that quantitatively is less than the established therapeutic dose for that particular pharmacological agent (e.g., as published in a reference consulted by a person of skill, for example, doses for a pharmacological agent published in the Physicians' Desk Reference, 69th Ed., 2015, PDR Network or Brunton, et al., Goodman & Gilman's The Pharmacological Basis of Therapeutics, 12th edition, 2010, McGraw-Hill Professional). A “subtherapeutic dose” can be defined in relative terms (i.e., as a percentage amount (less than 100%) of the amount of pharmacologically active agent conventionally administered). For example, a subtherapeutic dose amount can be about 1% to about 75% of the amount of pharmacologically active agent conventionally administered. In some embodiments, a subtherapeutic dose can be about 75%, 50%, 30%, 25%, 20%, 10% or less, than the amount of pharmacologically active agent conventionally administered.

The terms “controlled release,” “sustained release,” “extended release,” and “timed release” are intended to refer interchangeably to any drug-containing formulation in which release of the drug is not immediate, i.e., with a “controlled release” formulation, oral administration does not result in immediate release of the drug into an absorption pool. The terms are used interchangeably with “nonimmediate release” as defined in Remington: The Science and Practice of Pharmacy, University of the Sciences in Philadelphia, Eds., 21^{st} Ed., Lippencott Williams & Wilkins (2005).

The terms “sustained release” and “extended release” are used in their conventional sense to refer to a drug formulation that provides for gradual release of a drug over an extended period of time, for example, 12 hours or more, and that preferably, although not necessarily, results in substantially steady-state blood levels of a drug over an extended time period.

As used herein, the term “delayed release” refers to a pharmaceutical preparation that passes through the stomach intact and dissolves in the small intestine.

As used herein, “synergy” or “synergistic” interchangeably refer to the combined effects of two active agents that are greater than their additive effects. Synergy can also be achieved by producing an efficacious effect with combined inefficacious doses of two active agents. The measure of synergy is independent of statistical significance.

The terms “systemic administration” and “systemically administered” refer to a method of administering agent (e.g., an agent that reduces or inhibits ER stress, an agent that increases epoxygenated fatty acids (e.g., an inhibitor of sEH, an EET, an epoxygenated fatty acid, and mixtures thereof), optionally with an anti-inflammatory agent and/or an analgesic agent) to a mammal so that the agent/cells is delivered to sites in the body, including the targeted site of pharmaceutical action, via the circulatory system. Systemic administration includes, but is not limited to, oral, intranasal, rectal and parenteral (i.e., other than through the alimentary tract, such as intramuscular, intravenous, intra-arterial, transdermal and subcutaneous) administration.

The term “co-administration” refers to the presence of both active agents/cells in the blood or body at the same time. Active agents that are co-administered can be delivered concurrently (i.e., at the same time) or sequentially.

The phrase “cause to be administered” refers to the actions taken by a medical professional (e.g., a physician), or a person controlling medical care of a subject, that control and/or permit the administration of the agent(s)/compound(s)/cell(s) at issue to the subject. Causing to be administered can involve diagnosis and/or determination of an appropriate therapeutic or prophylactic regimen, and/or prescribing particular agent(s)/compounds/cell(s) for a subject. Such prescribing can include, for example, drafting a prescription form, annotating a medical record, and the like.

The terms “patient,” “subject” or “individual” interchangeably refers to a non-human mammal, including primates (e.g., macaque, pan troglodyte, pongo), a domesticated mammal (e.g., felines, canines), an agricultural mammal (e.g., bovine, ovine, porcine, equine) and a laboratory mammal or rodent (e.g., rattus, murine, lagomorpha, hamster).

The term “mitigating” refers to reduction or elimination of one or more symptoms of that pathology or disease, and/or a reduction in the rate or delay of onset or severity of one or more symptoms of that pathology or disease, and/or the prevention of that pathology or disease.

The terms “inhibiting,” “reducing,” “decreasing” refers to inhibiting the disease condition of interest (e.g., renal inflammation, fibrosis and/or failure, insulin resistance, pre-diabetes, diabetes) mammalian subject by a measurable amount using any method known in the art. For example, inflammation is inhibited, reduced or decreased if an indicator of inflammation, e.g., swelling, blood levels of prostaglandin PGE2, is at least about 10%, 20%, 30%, 50%, 80%, or 100% reduced, e.g., in comparison to the same inflammatory indicator prior to administration of an agent that increases epoxygenated fatty acids (e.g., an inhibitor of sEH, an EET, an epoxygenated fatty acid, and mixtures thereof). In some embodiments, the disease condition is inhibited, reduced or decreased by at least about 1-fold, 2-fold, 3-fold, 4-fold, or more in comparison to the fibrosis and/or inflammation prior to administration of the agent that increases epoxygenated fatty acids (e.g., an inhibitor of sEH, an EET, an epoxygenated fatty acid, and mixtures thereof). Indicators of renal inflammation and/or failure, insulin resistance, pre-diabetes and diabetes can also be qualitative.

As used herein, the phrase “consisting essentially of” refers to the genera or species of active pharmaceutical agents included in a method or composition, as well as any excipients inactive for the intended purpose of the methods or compositions. In some embodiments, the phrase “consisting essentially of” expressly excludes the inclusion of one or more additional active agents other than the listed active agents, e.g., an agent that increases epoxygenated fatty acids (e.g., an inhibitor of sEH, an EET, an epoxygenated fatty acid, and mixtures thereof) and/or an anti-inflammatory agent.

As used herein, the term “subject suspected of having diabetes” refers to a subject that presents one or more symptoms indicative of diabetes or a diabetes-related condition (e.g., diabetes mellitus type 1, diabetes mellitus type 2, gestational diabetes, pre-diabetes, metabolic syndrome, syndrome X) (e.g., polyuria, polydipsia, nocturia, fatigue, weight loss) or is being screened for diabetes (e.g., during a routine physical). A subject suspected of having diabetes or a diabetes-related condition may also have one or more risk factors. A subject suspected of having diabetes or a diabetes-related condition has generally not been tested for diabetes or a diabetes-related condition. However, a “subject suspected of having diabetes” encompasses an individual for whom a confirmatory test (e.g., fasting glucose plasma level) has not been done or for whom the type of diabetes is not known. A “subject suspected of having diabetes” is sometimes diagnosed with diabetes and is sometimes found to not have diabetes.

As used herein, the phrase “subject diagnosed with diabetes” refers to a subject who has been tested and found to have diabetes or a diabetes-related condition (e.g., diabetes mellitus type 1, diabetes mellitus type 2, gestational diabetes, pre-diabetes, metabolic syndrome, syndrome X) (e.g., a random plasma glucose level of ≥200 mg/dL or greater, a fasting glucose plasma level of ≥126 mg/dL occurring on two separate occasions, 2 hours post glucose load (75 g) plasma glucose of ≥200 mg/dL on two separate occasions). Diabetes may be diagnosed using any suitable method, including but not limited to, measurements of random plasma glucose level, fasting plasma glucose level, hemoglobin A1c (HbA1c or A1c) levels, glycosylated hemoglobin (GHb) levels. A subject having diabetes has hemoglobin A1c (HbA1c or A1c) levels above 6.4%, fasting plasma glucose levels of greater than or equal to 126 mg/dl (wherein fasting means not having anything to eat or drink (except water) for at least 8 hours before the test), and/or blood glucose levels of greater than or equal to 200 mg/dl in an oral glucose tolerance test (OGTT; a two-hour test that checks blood glucose levels before and 2 hours after the subject drinks a sweet drink). A “preliminary diagnosis” is one based only on presenting symptoms (e.g., polyuria, polydipsia, nocturia, fatigue, weight loss). See, www.diabetes.org.

As used herein, a “subject having pre-diabetes” has blood glucose levels that are higher than normal but not yet high enough to be diagnosed as diabetes. A subject having pre-diabetes has impaired glucose tolerance (IGT) and/or impaired fasting glucose (IFG). In quantitative diagnostic tests, a subject having pre-diabetes has hemoglobin A1c (HbA1c or A1c) levels in the range of 5.7% to 6.4%, fasting plasma glucose levels in the range of 100 mg/dl to 125 mg/dl (wherein fasting means not having anything to eat or drink (except water) for at least 8 hours before the test), and/or blood glucose levels in the range of 140 mg/dl to 199 mg/dl in an oral glucose tolerance test (OGTT). See, www.diabetes.org.

As used herein, the phrase “subject at risk for diabetes” refers to a subject with one or more risk factors for developing diabetes or a diabetes-related condition. Risk factors include, but are not limited to, obesity (particularly central or abdominal obesity), race, gender, age, genetic predisposition, diet, lifestyle (particularly sedentary lifestyle), and diseases or conditions that can lead to secondary diabetes (e.g., treatment with glucocorticoids, Cushing syndrome, acromegaly, pheochromocytoma).

As used herein, the phrase “characterizing diabetes in subject or patient or individual” refers to the identification of one or more properties of diabetes disease or a diabetes-related disease in a subject, including but not limited to, plasma glucose levels (random, fasting, or upon glucose challenge); Hemoglobin A1c (HbA1c or A1c) levels; glycosylated hemoglobin (GHb) levels; microalbumin levels or albumin-to-creatinine ratio; insulin levels; C-peptide levels; antibodies to insulin, islet cells, or glutamic acid decarboxylase (GAD); levels of anti-GAD65 antibody (e.g., as an indicator of latent autoimmune diabetes of adults).

### BRIEF DESCRIPTION OF THE DRAWINGS

FIGS. 1A-F illustrate efficient and specific deletion of sEH in podocytes. A) sEH expression is increased in podocytes under high fat and hyperglycemic conditions. Immunoblots of sEH and synaptopodin expression in total kidney lysates of male mice fed regular chow and HFD (for 3 and 6 months) and in mice at 3 months after STZ. B) Primary podocytes lysates of control (Ctrl) and pod-sEHKO (KO) mice fed regular chow and HFD for 3 months (top) and STZ-treated (bottom) were immunoblotted for sEH, synaptopodin and tubulin. C) sEH genomic locus and targeting design. Two loxP sites were designed in an intronic region of the sEH gene. D) Confirmation of sEH floxed and Cre mice by PCR. Genomic DNA from tails were amplified by PCR, primers were designed to distinguish the alleles with & without loxP insertions (left), and Cre (right). E) Immunoblots of sEH expression in lysates of primary podocytes, epididymal fat, liver and muscle of control (Ctrl) and pod-sEHKO (KO) mice. Representative immunoblots are shown. F) Immunostaining of nephrin (green) and sEH (red) in kidney paraffin section of Ctrl and KO mice. Scale bar: 200 μm.

FIGS. 2A-G illustrate improved blood pressure, insulin sensitivity and enhanced glucose tolerance in pod-sEHKO mice. A) Systolic (S) and diastolic (D) blood pressure were measured at week 20 post STZ in control (Ctrl) and pod-sEHKO (KO) mice. *p<0.05; **p<0.01 without vs with STZ, and †p<0.05; Ctrl+STZ vs KO+STZ. Insulin tolerance tests on control and KO mice without and with STZ at 2 (B) and 15 weeks (C) weeks after STZ injection. Glucose tolerance tests on control and KO mice without and with STZ at 3 (D) and 16 weeks (E) after STZ injection. Area under the curve (AUC) calculations for glucose tolerance was calculated. *p<0.05; **p<0.01 between the indicated time point and 0 min, and †p<0.05; ††p<0.01 Ctrl vs KO. Fed PEPCK (F) and G6pase (G) mRNA in liver and kidney of control and pod-sEHKO mice without and with STZ treatment.

FIGS. 3A-B illustrate podocyte sEH deficiency attenuates hyperglycemia-induced glomeruloscelerosis. A) PAS staining in paraffin-embedded section of kidneys from control (Ctrl) and pod-sEHKO (KO) mice without (−) and with (+) STZ at 24 weeks after injection. Arrowhead indicates flattened epithelia. Arrow indicates K-W nodules. Scale bar: 200 μm. B) TEM of podocytes from Ctrl and KO mice without (−) and with (+) STZ at 24 weeks after injection. Arrow indicates foot processes. Scale bar: 2 μm.

FIGS. 4A-B illustrate decreased endoplasmic reticulum stress and inflammation in pod-sEHKO mice. A) Immunoblots of pPERK (Thr⁹⁸⁰), PERK, peIF2α (Ser⁵¹), eIF2 α, pIRE1α (Ser724), IRE1α, spliced xBP1 (sXBP1) and Tubulin in total kidney lysates from Ctrl and KO mice without and with STZ at 24 weeks after injection. Each lane represents lysate from a different animal. Bar charts represent pPERK, peIF2α and pIRE1 normalized to the respective protein expression. B) Immunoblots of NFκB signaling proteins in total kidney lysates from Ctrl and KO mice without and with STZ at 24 weeks after injection. Bar charts normalized data for pIKKα/IKKα, pIKBα/IKBα, pNF-κB/NF-KB and NF-KBp50/Tubulin as means+SEM (A.U: arbitrary units). *p<0.05; **p<0.01 without vs with STZ, and †p<0.05; ††p<0.01 Ctrl vs KO.

FIG. 5A-B illustrate enhanced autophagy in pod-sEHKO mice. Immunoblots of key signaling proteins in the autophagy (pAMPK (Thr¹⁷²), AMPK, PGC1α, Beclin and LC3) and fibrosis (TGFβRII, pSmad2 (Ser⁴⁶⁵) and Smad2) pathways in total kidney lysates from Ctrl and KO mice without and with STZ at 24 weeks after injection. Each lane represents lysate form a different animal. Bar charts represent pAMPK and pSmad2 normalized to the respective protein expression and Beclin, LC3 and TGFβRII normalized to Tubulin as means+SEM (AU: arbitrary units). *p<0.05; **p<0.01 without vs with STZ, and †p<0.05; ††p<0.01 Ctrl vs KO.

FIG. 6 illustrates pharmacological inhibition of sEH in differentiated podocytes attenuates ER stress and enhances autophagy. Immunoblots of key signaling proteins in the ER stress pPERK (Thr⁹⁸⁰), PERK, pIRE1α (Ser⁷²⁴) and IRE1α, autophagy (Beclin and LC3) and fibrosis (TGFβRII, pSmad2 (Ser⁴⁶⁵) and Smad2) pathways in E11 podocytes cultured in low (5.6 mM) and high (25 mM) glucose concentrations for 72 h with or without TPPU (1 μM for 12 h), ER stress inhibitor 4-PBA (250 μM) or the autophagy inhibitor; DBeQ (15 μM). All inhibitors were added 12 h prior to cell harvest.

FIG. 7 illustrates that the mRNA of beclin, Lc3 and additional markers of autophagy cysteine protease ATG4D (Atg4) and Unc-51-like kinase 2 (Ulk2) were enhanced in pod-sEHKO mice under hyperglycemic conditions.

### DETAILED DESCRIPTION

1. Introduction

Provided are methods and compositions of treating diabetes and insulin resistance, e.g., by increasing glucose urine excretion, treating diabetic nephropathy, and improving blood pressure using soluble epoxide hydrolase inhibitors as monotherapy or in combination with other inhibitors.

The present compositions and methods are based, in part, on the investigation and discovery of the effects of podocyte specific sEH deletion on kidney function under normoglycemic and hyperglycemic conditions and the underlying molecular mechanisms. Diabetic nephropathy (DN) is the leading cause of renal failure and is characterized by proteinuria that progresses to renal inflammation and decline in glomerular filtration barrier (GFB). The podocyte is important in maintaining the integrity of GFB and podocyte dysfunction plays a significant role in the pathogenesis of DN. Soluble epoxide hydrolase (sEH) is a cytosolic enzyme whose inhibition has beneficial effects in inflammatory diseases, but its significance in podocytes remains unexplored. To determine whether sEH in podocytes affects renal function in vivo, we generated mice with podocyte-specific deletion of sEH (hereafter termed pod-sEHKO). These animals exhibit moderate improvement in kidney function and systemic glucose homeostasis in a normoglycemic environment but display significant improvement under hyperglycemic condition. Electron microscopy revealed that sEH deficiency protected podocyte structure and foot processes against hyperglycemia-induced toxicity. Moreover, podocyte sEH deficiency was associated with decreased endoplasmic reticulum stress and enhanced autophagy with corresponding decrease in inflammation and fibrosis in the kidney. These effects were likely cell-autonomous since they were recapitulated in differentiated mouse podocytes treated with sEH pharmacological inhibitor. Collectively, these findings identify sEH in podocytes as a key and significant contributor to kidney function and systemic glucose homeostasis which may have potential therapeutic implications.

The increasing prevalence of diabetes mellitus has become a global health issue and it is projected that the number of people with diabetes worldwide will increase from 382 million in 2013 to 592 by 2035. Diabetic nephropathy (DN) is one of the most devastating complications of diabetes and the leading cause of end stage kidney disease. DN begins with proteinuria then progresses to renal inflammation and decline in glomerular filtration barrier (GFB). GFB is composed of two cell types, podocytes and glomerular endothelial cells. The podocyte is particularly important in maintaining the integrity of GFB in humans, and there is growing evidence that podocyte dysfunction plays an important role in the pathogenesis of DN. Elucidating the mechanisms underlying podocyte function is critical for understanding disease pathogenesis and developing better therapies.

We generated mice with podocyte-specific deletion of sEH. These animals exhibit moderate improvement in kidney function and systemic glucose homeostasis in a normoglycemic environment but display significant improvement under hyperglycemic condition. Electron microscopy revealed that sEH deficiency protected podocyte structure and foot processes against hyperglycemia-induced toxicity. Moreover, podocyte sEH deficiency was associated with decreased endoplasmic reticulum stress and enhanced autophagy with corresponding decrease in inflammation and fibrosis in the kidney. These effects were likely cell-autonomous since they were recapitulated in differentiated mouse podocytes treated with selective sEH pharmacological inhibitor. Collectively, these findings identify sEH in podocytes as a key and significant contributor to kidney function. Importantly, these novel and unexpected findings demonstrate that sEH inhibitors (pharmacological and gene-based) can be deployed to decrease blood glucose levels in diabetes by increasing glucose clearance in urine. In addition, these inhibitors will improve glucose homeostasis in insulin-resistant pre-diabetic state. Moreover, sEH inhibitors improve kidney function and blood pressure and protect podocytes from hyperglycemia-induced injury. Finally, these inhibitors have additional salutary effects by increasing serum HDL levels under hyperglycemic conditions.

2. Subjects Who May Benefit—Conditions Subject to Treatment

Subjects who may benefit generally have symptomatic renal dysfunction or impaired renal function. For example, the subject may suffer congenital or chronic nephropathy. In varying embodiments, the nephropathy is secondary to or caused by diabetes, e.g., the subject has or is suspected of having diabetic kidney disease (DKD). In varying embodiments subjects who may benefit have pre-diabetes or diabetes, or be suspected of having pre-diabetes or diabetes. In varying embodiments, the subject may be exhibiting symptoms of renal dysfunction or reduced renal function or renal failure. For example, in varying embodiments, the subject may be exhibiting one or more symptoms of impaired renal function, including proteinuria, renal inflammation and/or decline in glomerular filtration barrier (GFB).

In varying embodiments, the subject is a child, a juvenile or an adult. In varying embodiments, the subject is a mammal, for example, a human or a domesticated mammal (e.g., a canine or a feline).

3. Agents that Reduce and/or Inhibit Endoplasmic Reticular (ER) Stress

Methods and compositions described herein involve the co-formulation and/or co-administration of an agent that increases the production and/or level of epoxygenated fatty acids and an inhibitor of endoplasmic reticular (ER) stress. Any agent known in the art to reduce and/or inhibit levels of ER stress can be used. Illustrative agents that reduce and/or inhibit ER stress include without limitation, e.g., 4-phenyl butyric acid (“PBA”), butyrate, 3-phenylpropionic acid (3-PPA), 5-phenylvaleric acid (5-PVA), 6 phenylhexanoic acid (6-PHA), dimethyl-celecoxib (DMCx), tauroursodeoxycholic acid, trehalose, deuterated water, docosahexaenoic acid (“DHA”), eicosapentaenoic acid (“EPA”), vitamin C, arabitol, mannose, glycerol, betaine, sarcosine, trimethylamine-N oxide and DMSO.

4. Agents that Increase the Production and/or Level of Epoxygenated Fatty Acids

Agents that increase epoxygenated fatty acids include epoxygenated fatty acids (e.g., including EETs), and inhibitors of soluble epoxide hydrolase (sEH).

a. Inhibitors of Soluble Epoxide Hydrolase (sEH)

Scores of sEH inhibitors are known, of a variety of chemical structures. Derivatives in which the urea, carbamate or amide pharmacophore are particularly useful as sEH inhibitors. As used herein, “pharmacophore” refers to the section of the structure of a ligand that binds to the sEH. In various embodiments, the urea, carbamate or amide pharmacophore is covalently bound to both an adamantane and to a 12 carbon chain dodecane. Derivatives that are metabolically stable are preferred, as they are expected to have greater activity in vivo. Selective and competitive inhibition of sEH in vitro by a variety of urea, carbamate, and amide derivatives is taught, for example, by Morisseau et al., Proc. Natl. Acad. Sci. U.S.A., 96:8849-8854 (1999), which provides substantial guidance on designing urea derivatives that inhibit the enzyme.

Derivatives of urea are transition state mimetics that form a preferred group of sEH inhibitors. Within this group, N, N′-dodecyl-cyclohexyl urea (DCU), is preferred as an inhibitor, while N-cyclohexyl-N′-dodecylurea (CDU) is particularly preferred. Some compounds, such as dicyclohexylcarbodiimide (a lipophilic diimide), can decompose to an active urea inhibitor such as DCU. Any particular urea derivative or other compound can be easily tested for its ability to inhibit sEH by standard assays, such as those discussed herein. The production and testing of urea and carbamate derivatives as sEH inhibitors is set forth in detail in, for example, Morisseau et al., Proc Natl Acad Sci (USA) 96:8849-8854 (1999).

N-Adamantyl-N′-dodecyl urea (“ADU”) is both metabolically stable and has particularly high activity on sEH. (Both the 1- and the 2-admamantyl ureas have been tested and have about the same high activity as an inhibitor of sEH. Thus, isomers of adamantyl dodecyl urea are preferred inhibitors. It is further expected that N, N′-dodecyl-cyclohexyl urea (DCU), and other inhibitors of sEH, and particularly dodecanoic acid ester derivatives of urea, are suitable for use in the methods. Preferred inhibitors include:
12-(3-Adamantan-1-yl-ureido)dodecanoic acid (AUDA),

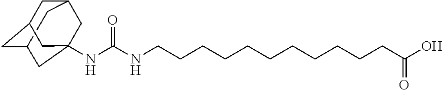 [SMILES: O=C(O)CCCCCCCCCCCNC(=O)NC12CC3CC(CC(C3)C1)C2]

12-(3-Adamantan-1-yl-ureido)dodecanoic acid butyl ester (AUDA-BE),

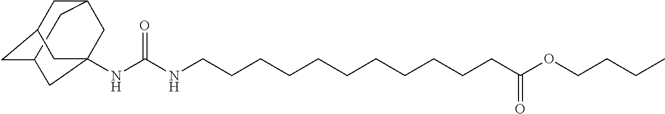 [SMILES: CCCCOC(=O)CCCCCCCCCCCNC(=O)NC12CC3CC(CC(C3)C1)C2]

Adamantan-1-yl-3-{5-[2-(2-ethoxyethoxy)ethoxy]pentyl}urea (compound 950, also referred to herein as “AEPU”), and

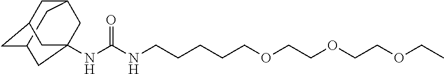 [SMILES: CCOCCOCCOCCCCCNC(=O)NC12CC3CC(CC(C3)C1)C2]

Another preferred group of inhibitors are piperidines. The following Tables sets forth some exemplar inhibitors of sEH and their ability to inhibit sEH activity of the human enzyme and sEH from equine, ovine, porcine, feline and canine, expressed as the amount needed to reduce the activity of the enzyme by 50% (expressed as “IC₅₀”).

TABLE 1
IC₅₀ values for selected alkylpiperidine-based sEH inhibitors against human sEH

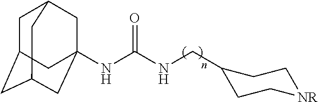

| | | n = 0 | | n = 1 | |
|---|---|---|---|---|---|
| | | Compound | IC₅₀ (μM)^{a} | Compound | IC₅₀ (μM)^{a} |
| R: | H | I | 0.30 | II | 4.2 |
| | 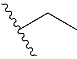 [SMILES: *CC] | 3a | 3.8 | 4.a | 3.9 |
| | 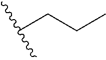 [SMILES: *CCC] | 3b | 0.81 | 4b | 2.6 |
| | 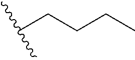 [SMILES: *CCCC] | 3c | 1.2 | 4c | 0.61 |
| | 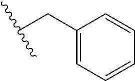 [SMILES: *Cc1ccccc1] | 3d | 0.01 | 4d | 0.11 |

^{a}As determined via a kinetic fluorescent assay.

TABLE 2
sEH inhibitors

| Structure | Name | sEHi # |
|---|---|---|
| 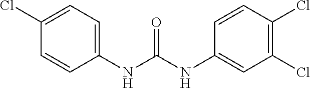 [SMILES: O=C(Nc1ccc(Cl)cc1)Nc1ccc(Cl)c(Cl)c1] | 3-(4-chlorophenyl)-1-(3,4-dichlorophenyl)urea or 3,4,4′-trichlorocarbanilide | 295 (TCC) |
| 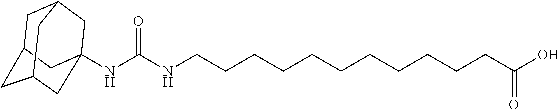 [SMILES: O=C(O)CCCCCCCCCCCNC(=O)NC12CC3CC(CC(C3)C1)C2] | 12-(3-adamantan-1-yl-ureido)dodecanoic acid | 700 (AUDA) |
| 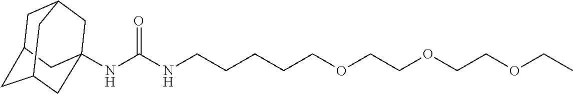 [SMILES: CCOCCOCCOCCCCCNC(=O)NC12CC3CC(CC(C3)C1)C2] | 1-adamantanyl-3-{5-[2-(2-ethoxyethoxy)ethoxy]pentyl}urea | 950 (AEPU) |
| 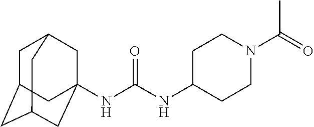 [SMILES: CC(=O)N1CCC(NC(=O)NC23CC4CC(CC(C4)C2)C3)CC1] | 1-(1-acetypiperidin-4-yl)-3-adamantanylurea | 1153 (APAU) |
| 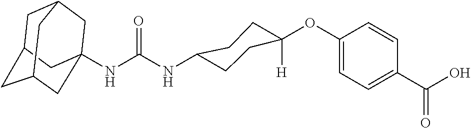 [SMILES: O=C(NC1CCC(Oc2ccc(C(=O)O)cc2)CC1)NC12CC3CC(CC(C3)C1)C2] | trans-4-[4-(3-Aamantan-1-yl-ureido)-cyclohexyloxy]-benzoic acid | 1471 (tAUCB) |
| 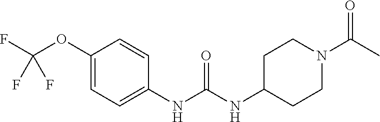 [SMILES: CC(=O)N1CCC(NC(=O)Nc2ccc(OC(F)(F)F)cc2)CC1] | 1-trifluoromethoxyphenyl-3-(1-acetylpiperidin-4-yl)urea | 1555 (TPAU) |
| 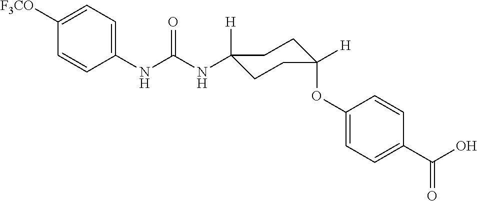 [SMILES: O=C(Nc1ccc(OC(F)(F)F)cc1)NC1CCC(Oc2ccc(C(=O)O)cc2)CC1] | cis-4-[4-(3-Adamantan-1-yl-ureido)-cyclohexyloxy]-benzoic acid | 1686 (cAUCB) |
| 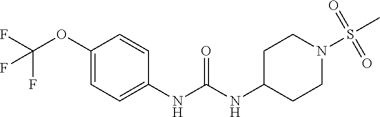 [SMILES: CS(=O)(=O)N1CCC(NC(=O)Nc2ccc(OC(F)(F)F)cc2)CC1] | 1-(1-methylsulfonyl-piperidin-4-yl)-3-(4-trifluoromethoxy-phenyl)-urea | 1709 (TUPS) |
| 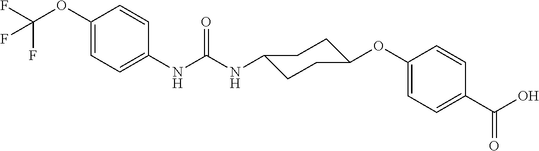 [SMILES: O=C(Nc1ccc(OC(F)(F)F)cc1)NC1CCC(Oc2ccc(C(=O)O)cc2)CC1] | trans-4-{4-[3-(4-Trifluoromethoxy-phenyl)-ureido]-cyclohexyloxy}-benzoic acid | 1728 (tTUCB) |
| 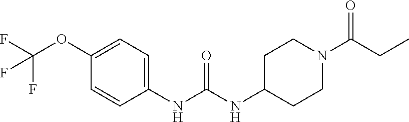 [SMILES: CCC(=O)N1CCC(NC(=O)Nc2ccc(OC(F)(F)F)cc2)CC1] | 1-trifluoromethoxyphenyl-3-(1-propionylpiperidin-4-yl)urea | 1770 (TPPU) |
| 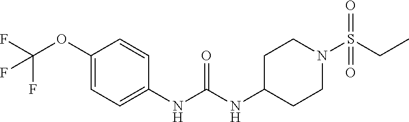 [SMILES: CCS(=O)(=O)N1CCC(NC(=O)Nc2ccc(OC(F)(F)F)cc2)CC1] | 1-(1-ethylsulfonyl-piperidin-4-yl)-3-(4-trifluoromethoxy-phenyl)-urea | 2213 (TUPSE) |
| 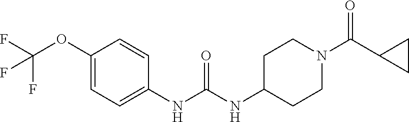 [SMILES: O=C(Nc1ccc(OC(F)(F)F)cc1)NC1CCN(C(=O)C2CC2)CC1] | 1-(1-(cyclopropanecarbonyl)piperidin-4-yl)-3-(4-(trifluoromethoxy)phenyl)urea | 2214 (CPTU) |
| 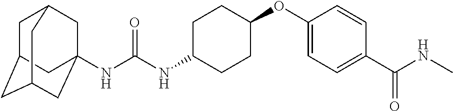 | trans-N-methyl-4-[4-(3-Adamantan-1-yl-ureido)-cyclohexyloxy]-benzamide | 2225 (tMAUCB) |
| 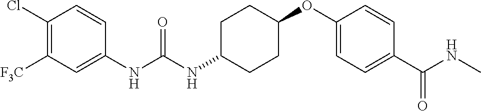 | trans-N-methyl-4-[4-((3-trifluoromethyl-4-chlorophenyl)-ureido)-cyclohexyloxy]-benzamide | 2226 (tMTCUCB) |
| 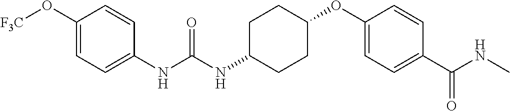 | cis-N-methyl-4-{4-[3-(4-trifluoromethoxy-phenyl)-ureido]-cyclohexyloxy}-benzamide | 2228 (cMTUCB) |
| 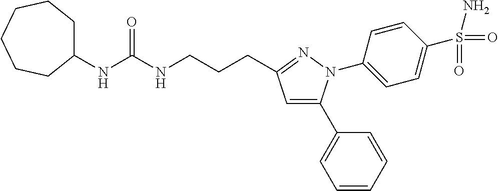 [SMILES: NS(=O)(=O)c1ccc(-n2nc(CCCNC(=O)NC3CCCCCC3)cc2-c2ccccc2)cc1] | 1-cycloheptyl-3-(3-(1,5-diphenyl-1H-pyrazol-3-yl)propyl)urea | 2247 (HDP3U) |

A number of other sEH inhibitors which can be used in the methods and compositions are set forth in co-owned applications PCT/US2013/024396, PCT/US2012/025074, PCT/US2011/064474, PCT/US2011/022901, PCT/US2008/072199, PCT/US2007/006412, PCT/US2005/038282, PCT/US2005/08765, PCT/US2004/010298 and U.S. Published Patent Application Publication Nos: 2014/0088156, 2014/0038923, 2013/0274476, 2013/0143925, 2013/0137726, 2011/0098322, 2005/0026844, each of which is hereby incorporated herein by reference in its entirety for all purposes.

U.S. Pat. No. 5,955,496 (the '496 patent) also sets forth a number of sEH inhibitors which can be used in the methods. One category of these inhibitors comprises inhibitors that mimic the substrate for the enzyme. The lipid alkoxides (e.g., the 9-methoxide of stearic acid) are an exemplar of this group of inhibitors. In addition to the inhibitors discussed in the '496 patent, a dozen or more lipid alkoxides have been tested as sEH inhibitors, including the methyl, ethyl, and propyl alkoxides of oleic acid (also known as stearic acid alkoxides), linoleic acid, and arachidonic acid, and all have been found to act as inhibitors of sEH.

In another group of embodiments, the '496 patent sets forth sEH inhibitors that provide alternate substrates for the enzyme that are turned over slowly. Exemplars of this category of inhibitors are phenyl glycidols (e.g., S, S-4-nitrophenylglycidol), and chalcone oxides. The '496 patent notes that suitable chalcone oxides include 4-phenylchalcone oxide and 4-fluourochalcone oxide. The phenyl glycidols and chalcone oxides are believed to form stable acyl enzymes.

Additional inhibitors of sEH suitable for use in the methods are set forth in U.S. Pat. No. 6,150,415 (the '415 patent) and U.S. Pat. No. 6,531,506 (the '506 patent). Two preferred classes of sEH inhibitors are compounds of Formulas 1 and 2, as described in the '415 and '506 patents. Means for preparing such compounds and assaying desired compounds for the ability to inhibit epoxide hydrolases are also described. The '506 patent, in particular, teaches scores of inhibitors of Formula 1 and some twenty sEH inhibitors of Formula 2, which were shown to inhibit human sEH at concentrations as low as 0.1 μM. Any particular sEH inhibitor can readily be tested to determine whether it will work in the methods by standard assays. Esters and salts of the various compounds discussed above or in the cited patents, for example, can be readily tested by these assays for their use in the methods.

As noted above, chalcone oxides can serve as an alternate substrate for the enzyme. While chalcone oxides have half-lives which depend in part on the particular structure, as a group the chalcone oxides tend to have relatively short half-lives (a drug's half-life is usually defined as the time for the concentration of the drug to drop to half its original value. See, e.g., Thomas, G., Medicinal Chemistry: an introduction, John Wiley & Sons Ltd. (West Sussex, England, 2000)). Since the various uses contemplate inhibition of sEH over differing periods of time which can be measured in days, weeks, or months, chalcone oxides, and other inhibitors which have a half-life whose duration is shorter than the practitioner deems desirable, are preferably administered in a manner which provides the agent over a period of time. For example, the inhibitor can be provided in materials that release the inhibitor slowly. Methods of administration that permit high local concentrations of an inhibitor over a period of time are known, and are not limited to use with inhibitors which have short half-lives although, for inhibitors with a relatively short half-life, they are a preferred method of administration.

In addition to the compounds in Formula 1 of the '506 patent, which interact with the enzyme in a reversible fashion based on the inhibitor mimicking an enzyme-substrate transition state or reaction intermediate, one can have compounds that are irreversible inhibitors of the enzyme. The active structures such as those in the Tables or Formula 1 of the '506 patent can direct the inhibitor to the enzyme where a reactive functionality in the enzyme catalytic site can form a covalent bond with the inhibitor. One group of molecules which could interact like this would have a leaving group such as a halogen or tosylate which could be attacked in an SN2 manner with a lysine or histidine. Alternatively, the reactive functionality could be an epoxide or Michael acceptor such as an α/β-unsaturated ester, aldehyde, ketone, ester, or nitrile.

Further, in addition to the Formula 1 compounds, active derivatives can be designed for practicing the invention. For example, dicyclohexyl thio urea can be oxidized to dicyclohexylcarbodiimide which, with enzyme or aqueous acid (physiological saline), will form an active dicyclohexylurea. Alternatively, the acidic protons on carbamates or ureas can be replaced with a variety of substituents which, upon oxidation, hydrolysis or attack by a nucleophile such as glutathione, will yield the corresponding parent structure. These materials are known as prodrugs or protoxins (Gilman et al., The Pharmacological Basis of Therapeutics, 7th Edition, MacMillan Publishing Company, New York, p. 16 (1985)) Esters, for example, are common prodrugs which are released to give the corresponding alcohols and acids enzymatically (Yoshigae et al., Chirality, 9:661-666 (1997)). The drugs and prodrugs can be chiral for greater specificity. These derivatives have been extensively used in medicinal and agricultural chemistry to alter the pharmacological properties of the compounds such as enhancing water solubility, improving formulation chemistry, altering tissue targeting, altering volume of distribution, and altering penetration. They also have been used to alter toxicology profiles.

There are many prodrugs possible, but replacement of one or both of the two active hydrogens in the ureas described here or the single active hydrogen present in carbamates is particularly attractive. Such derivatives have been extensively described by Fukuto and associates. These derivatives have been extensively described and are commonly used in agricultural and medicinal chemistry to alter the pharmacological properties of the compounds. (Black et al., Journal of Agricultural and Food Chemistry, 21(5):747-751 (1973); Fahmy et al, Journal of Agricultural and Food Chemistry, 26(3):550-556 (1978); Jojima et al., Journal of Agricultural and Food Chemistry, 31(3):613-620 (1983); and Fahmy et al., Journal of Agricultural and Food Chemistry, 29(3):567-572 (1981).)

Such active proinhibitor derivatives are within the scope of the present invention, and the just-cited references are incorporated herein by reference. Without being bound by theory, it is believed that suitable inhibitors mimic the enzyme transition state so that there is a stable interaction with the enzyme catalytic site. The inhibitors appear to form hydrogen bonds with the nucleophilic carboxylic acid and a polarizing tyrosine of the catalytic site.

In some embodiments, the sEH inhibitor used in the methods taught herein is a “soft drug.” Soft drugs are compounds of biological activity that are rapidly inactivated by enzymes as they move from a chosen target site. EETs and simple biodegradable derivatives administered to an area of interest may be considered to be soft drugs in that they are likely to be enzymatically degraded by sEH as they diffuse away from the site of interest following administration. Some sEHI, however, may diffuse or be transported following administration to regions where their activity in inhibiting sEH may not be desired. Thus, multiple soft drugs for treatment have been prepared. These include but are not limited to carbamates, esters, carbonates and amides placed in the sEHI, approximately 7.5 angstroms from the carbonyl of the central pharmacophore. These are highly active sEHI that yield biologically inactive metabolites by the action of esterase and/or amidase. Groups such as amides and carbamates on the central pharmacophores can also be used to increase solubility for applications in which that is desirable in forming a soft drug. Similarly, easily metabolized ethers may contribute soft drug properties and also increase the solubility.

In some embodiments, sEH inhibition can include the reduction of the amount of sEH. As used herein, therefore, sEH inhibitors can therefore encompass nucleic acids that inhibit expression of a gene encoding sEH. Many methods of reducing the expression of genes, such as reduction of transcription and siRNA, are known, and are discussed in more detail below.

In various embodiments, a compound with combined functionality to concurrently inhibit sEH and COX-2 is administered. Urea-containing pyrazoles that function as dual inhibitors of cyclooxygenase-2 and soluble epoxide hydrolase are described, e.g., in Hwang, et al., J Med Chem. (2011) 28; 54(8):3037-50.

Preferably, the inhibitor inhibits sEH without also significantly inhibiting microsomal epoxide hydrolase (“mEH”). Preferably, at concentrations of 100 μM, the inhibitor inhibits sEH activity by at least 50% while not inhibiting mEH activity by more than 10%. Preferred compounds have an IC₅₀ (inhibition potency or, by definition, the concentration of inhibitor which reduces enzyme activity by 50%) of less than about 100 μM. Inhibitors with IC₅₀s of less than 100 μM are preferred, with IC₅₀s of less than 75 μM being more preferred and, in order of increasing preference, an IC₅₀ of 50 μM, 40 μM, 30 μM, 25 μM, 20 μM, 15 μM, 10 μM, 5 μM, 3 μM, 2 μM, 1 μM, 100 nM, 10 nM, 1.0 nM, or even less, being still more preferred. Assays for determining sEH activity are known in the art and described elsewhere herein. The IC₅₀ determination of the inhibitor can be made with respect to an sEH enzyme from the species subject to treatment (e.g., the subject receiving the inhibitor of sEH).

b. Cis-Epoxyeicosantrienoic Acids (“EETs”)

EETs, which are epoxides of arachidonic acid, are known to be effectors of blood pressure, regulators of inflammation, and modulators of vascular permeability. Hydrolysis of the epoxides by sEH diminishes this activity. Inhibition of sEH raises the level of EETs since the rate at which the EETs are hydrolyzed into dihydroxyeicosatrienoic acids (“DHETs”) is reduced.

It has long been believed that EETs administered systemically would be hydrolyzed too quickly by endogenous sEH to be helpful. For example, in one prior report of EETs administration, EETs were administered by catheters inserted into mouse aortas. The EETs were infused continuously during the course of the experiment because of concerns over the short half-life of the EETs. See, Liao and Zeldin, International Publication WO 01/10438 (hereafter “Liao and Zeldin”). It also was not known whether endogenous sEH could be inhibited sufficiently in body tissues to permit administration of exogenous EET to result in increased levels of EETs over those normally present. Further, it was thought that EETs, as epoxides, would be too labile to survive the storage and handling necessary for therapeutic use.

Studies from the laboratory of the present inventors, however, showed that systemic administration of EETs in conjunction with inhibitors of sEH had better results than did administration of sEH inhibitors alone. EETs were not administered by themselves in these studies since it was anticipated they would be degraded too quickly to have a useful effect. Additional studies from the laboratory of the present inventors have since shown, however, that administration of EETs by themselves has had therapeutic effect. Without wishing to be bound by theory, it is surmised that the exogenous EET overwhelms endogenous sEH, and allows EETs levels to be increased for a sufficient period of time to have therapeutic effect. Thus, EETs can be administered without also administering an sEHI to provide a therapeutic effect. Moreover, EETs, if not exposed to acidic conditions or to sEH are stable and can withstand reasonable storage, handling and administration.

In short, sEHI, EETs, or co-administration of sEHIs and of EETs, can be used in the methods of the present invention. In some embodiments, one or more EETs are administered to the patient without also administering an sEHI. In some embodiments, one or more EETs are administered shortly before or concurrently with administration of an sEH inhibitor to slow hydrolysis of the EET or EETs. In some embodiments, one or more EETs are administered after administration of an sEH inhibitor, but before the level of the sEHI has diminished below a level effective to slow the hydrolysis of the EETs.

EETs useful in the methods of the present invention include 14,15-EET, 8,9-EET and 11,12-EET, and 5,6 EETs. Preferably, the EETs are administered as the methyl ester, which is more stable. Persons of skill will recognize that the EETs are regioisomers, such as 8S,9R- and 14R,15S-EET. 8,9-EET, 11,12-EET, and 14R,15S-EET, are commercially available from, for example, Sigma-Aldrich (catalog nos. E5516, E5641, and E5766, respectively, Sigma-Aldrich Corp., St. Louis, Mo.).

If desired, EETs, analogs, or derivatives that retain activity can be used in place of or in combination with unmodified EETs. Liao and Zeldin, supra, define EET analogs as compounds with structural substitutions or alterations in an EET, and include structural analogs in which one or more EET olefins are removed or replaced with acetylene or cyclopropane groups, analogs in which the epoxide moiety is replaced with oxitane or furan rings and heteroatom analogs. In other analogs, the epoxide moiety is replaced with ether, alkoxides, urea, amide, carbamate, difluorocycloprane, or carbonyl, while in others, the carboxylic acid moiety is stabilized by blocking beta oxidation or is replaced with a commonly used mimic, such as a nitrogen heterocycle, a sulfonamide, or another polar functionality. In preferred forms, the analogs or derivatives are relatively stable as compared to an unmodified EET because they are more resistant than an unmodified EET to sEH and to chemical breakdown. “Relatively stable” means the rate of hydrolysis by sEH is at least 25% less than the hydrolysis of the unmodified EET in a hydrolysis assay, and more preferably 50% or more lower than the rate of hydrolysis of an unmodified EET. Liao and Zeldin show, for example, episulfide and sulfonamide EETs derivatives. Amide and ester derivatives of EETs and that are relatively stable are preferred embodiments. Whether or not a particular EET analog or derivative has the biological activity of the unmodified EET can be readily determined by using it in standard assays, such as radio-ligand competition assays to measure binding to the relevant receptor. As mentioned in the Definition section, above, for convenience of reference, the term “EETs” as used herein refers to unmodified EETs, and EETs analogs and derivatives unless otherwise required by context.

In some embodiments, the EET or EETs are embedded or otherwise placed in a material that releases the EET over time. Materials suitable for promoting the slow release of compositions such as EETs are known in the art. Optionally, one or more sEH inhibitors may also be placed in the slow release material.

Conveniently, the EET or EETs can be administered orally. Since EETs are subject to degradation under acidic conditions, EETs intended for oral administration can be coated with a coating resistant to dissolving under acidic conditions, but which dissolve under the mildly basic conditions present in the intestines. Suitable coatings, commonly known as “enteric coatings” are widely used for products, such as aspirin, which cause gastric distress or which would undergo degradation upon exposure to gastric acid. By using coatings with an appropriate dissolution profile, the coated substance can be released in a chosen section of the intestinal tract. For example, a substance to be released in the colon is coated with a substance that dissolves at pH 6.5-7, while substances to be released in the duodenum can be coated with a coating that dissolves at pH values over 5.5. Such coatings are commercially available from, for example, Rohm Specialty Acrylics (Rohm America LLC, Piscataway, N.J.) under the trade name “Eudragit®”. The choice of the particular enteric coating is not critical to the practice.

c. Phosphodiesterase Inhibitors (PDEi)

Phosphodiesterase inhibitors (PDEi) are well known anti-inflammatory agents. Many different classes of isozyme selective PDEi lead to remarkable increases in the plasma levels of a broad range of epoxy-fatty acids (EFA). The magnitude of this increase is so dramatic that PDEi can elevate epoxy-fatty acids as well as highly potent inhibitors of soluble epoxide hydrolase. Accordingly, levels of epoxygenated fatty acids (e.g., in blood, plasma, serum) can be increased by administration of a phosphodiesterase inhibitor (PDEi).

The PDEi may or may not be selective, specific or preferential for cAMP. Exemplary PDEs that degrade cAMP include without limitation PDE3, PDE4, PDE7, PDE8 and PDE10. Exemplary cAMP selective hydrolases include PDE4, 7 and 8. Exemplary PDEs that hydrolyse both cAMP and cGMP include PDE1, PDE2, PDE3, PDE10 and PDE11. Isoenzymes and isoforms of PDEs are well known in the art. See, e.g., Boswell-Smith et al., Brit. J. Pharmacol. 147:S252-257 (2006), and Reneerkens, et al., Psychopharmacology (2009) 202:419-443, the contents of which are incorporated herein by reference.

In some embodiments, the PDE inhibitor is a non-selective inhibitor of PDE. Exemplary non-selective PDE inhibitors that find use include without limitation caffeine, theophylline, isobutylmethylxanthine, aminophylline, pentoxifylline, vasoactive intestinal peptide (VIP), secretin, adrenocorticotropic hormone, pilocarpine, alpha-melanocyte stimulating hormone (MSH), beta-MSH, gamma-MSH, the ionophore A23187, prostaglandin E1.

In some embodiments, the PDE inhibitor used specifically or preferentially inhibits PDE4. Exemplary inhibitors that selectively inhibit PDE4 include without limitation rolipram, roflumilast, cilomilast, ariflo, HT0712, ibudilast and mesembrine.

In some embodiments, the PDE inhibitor used specifically or preferentially inhibits a cAMP PDE, e.g., PDE4, PDE7 or PDE8. In some embodiments, the PDE inhibitor used inhibits a cAMP PDE, e.g., PDE1, PDE2, PDE3, PDE4, PDE7, PDE8, PDE10 or PDE11. Exemplary agents that inhibit a cAMP phosphodiesterase include without limitation rolipram, roflumilast, cilomilast, ariflo, HT0712, ibudilast, mesembrine, cilostamide, enoxamone, milrinone, siguazodan and BRL-50481.

In some embodiments, the PDE inhibitor used specifically inhibits PDE5. Exemplary inhibitors that selectively inhibit PDE5 include without limitation sildenafil, zaprinast, tadalafil, udenafil, avanafil and vardenafil.

d. Assays for Epoxide Hydrolase Activity

Any of a number of standard assays for determining epoxide hydrolase activity can be used to determine inhibition of sEH. For example, suitable assays are described in Gill, et al., Anal Biochem 131:273-282 (1983); and Borhan, et al., Analytical Biochemistry 231:188-200 (1995)). Suitable in vitro assays are described in Zeldin et al., J Biol. Chem. 268:6402-6407 (1993). Suitable in vivo assays are described in Zeldin et al., Arch Biochem Biophys 330:87-96 (1996). Assays for epoxide hydrolase using both putative natural substrates and surrogate substrates have been reviewed (see, Hammock, et al. In: Methods in Enzymology, Volume III, Steroids and Isoprenoids, Part B, (Law, J. H. and H. C. Rilling, eds. 1985), Academic Press, Orlando, Fla., pp. 303-311 and Wixtrom et al., In: Biochemical Pharmacology and Toxicology, Vol. 1: Methodological Aspects of Drug Metabolizing Enzymes, (Zakim, D. and D. A. Vessey, eds. 1985), John Wiley & Sons, Inc., New York, pp. 1-93. Several spectral based assays exist based on the reactivity or tendency of the resulting diol product to hydrogen bond (see, e.g., Wixtrom, supra, and Hammock. Anal. Biochem. 174:291-299 (1985) and Dietze, et al. Anal. Biochem. 216:176-187 (1994)).

The enzyme also can be detected based on the binding of specific ligands to the catalytic site which either immobilize the enzyme or label it with a probe such as dansyl, fluoracein, luciferase, green fluorescent protein or other reagent. The enzyme can be assayed by its hydration of EETs, its hydrolysis of an epoxide to give a colored product as described by Dietze et al., 1994, supra, or its hydrolysis of a radioactive surrogate substrate (Borhan et al., 1995, supra). The enzyme also can be detected based on the generation of fluorescent products following the hydrolysis of the epoxide. Numerous methods of epoxide hydrolase detection have been described (see, e.g., Wixtrom, supra).

The assays are normally carried out with a recombinant enzyme following affinity purification. They can be carried out in crude tissue homogenates, cell culture or even in vivo, as known in the art and described in the references cited above.

e. Other Means of Inhibiting sEH Activity

Other means of inhibiting sEH activity or gene expression can also be used in the methods. For example, a nucleic acid molecule complementary to at least a portion of the human sEH gene can be used to inhibit sEH gene expression. Means for inhibiting gene expression using short RNA molecules, for example, are known. Among these are short interfering RNA (siRNA), small temporal RNAs (stRNAs), and micro-RNAs (miRNAs). Short interfering RNAs silence genes through a mRNA degradation pathway, while stRNAs and miRNAs are approximately 21 or 22 nt RNAs that are processed from endogenously encoded hairpin-structured precursors, and function to silence genes via translational repression. See, e.g., McManus et al., RNA, 8(6):842-50 (2002); Morris et al., Science, 305(5688):1289-92 (2004); He and Hannon, Nat Rev Genet. 5(7):522-31 (2004).

“RNA interference,” a form of post-transcriptional gene silencing (“PTGS”), describes effects that result from the introduction of double-stranded RNA into cells (reviewed in Fire, A. Trends Genet 15:358-363 (1999); Sharp, P. Genes Dev 13:139-141 (1999); Hunter, C. Curr Biol 9:R440-R442 (1999); Baulcombe. D. Curr Biol 9:R599-R601 (1999); Vaucheret et al. Plant J 16: 651-659 (1998)). RNA interference, commonly referred to as RNAi, offers a way of specifically inactivating a cloned gene, and is a powerful tool for investigating gene function.

The active agent in RNAi is a long double-stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown at this time. While RNAi was shown initially to work well in lower eukaryotes, for mammalian cells, it was thought that RNAi might be suitable only for studies on the oocyte and the preimplantation embryo.

In mammalian cells other than these, however, longer RNA duplexes provoked a response known as “sequence non-specific RNA interference,” characterized by the non-specific inhibition of protein synthesis.

Further studies showed this effect to be induced by dsRNA of greater than about 30 base pairs, apparently due to an interferon response. It is thought that dsRNA of greater than about 30 base pairs binds and activates the protein PKR and 2′,5′-oligonucleotide synthetase (2′,5′-AS). Activated PKR stalls translation by phosphorylation of the translation initiation factors eIF2α, and activated 2′,5′-AS causes mRNA degradation by 2′,5′-oligonucleotide-activated ribonuclease L. These responses are intrinsically sequence-nonspecific to the inducing dsRNA; they also frequently result in apoptosis, or cell death. Thus, most somatic mammalian cells undergo apoptosis when exposed to the concentrations of dsRNA that induce RNAi in lower eukaryotic cells.

More recently, it was shown that RNAi would work in human cells if the RNA strands were provided as pre-sized duplexes of about 19 nucleotide pairs, and RNAi worked particularly well with small unpaired 3′ extensions on the end of each strand (Elbashir et al. Nature 411: 494-498 (2001)). In this report, siRNA were applied to cultured cells by transfection in oligofectamine micelles. These RNA duplexes were too short to elicit sequence-nonspecific responses like apoptosis, yet they efficiently initiated RNAi. Many laboratories then tested the use of siRNA to knock out target genes in mammalian cells. The results demonstrated that siRNA works quite well in most instances.

For purposes of reducing the activity of sEH, siRNAs to the gene encoding sEH can be specifically designed using computer programs. The cloning, sequence, and accession numbers of the human sEH sequence are set forth in Beetham et al., Arch. Biochem. Biophys. 305(1):197-201 (1993). An exemplary amino acid sequence of human sEH (GenBank Accession No. L05779; SEQ ID NO:1) and an exemplary nucleotide sequence encoding that amino acid sequence (GenBank Accession No. AAA02756; SEQ ID NO:2) are set forth in U.S. Pat. No. 5,445,956. The nucleic acid sequence of human sEH is also published as GenBank Accession No. NM_001979.4; the amino acid sequence of human sEH is also published as GenBank Accession No. NP_001970.2.

A program, siDESIGN from Dharmacon, Inc. (Lafayette, Colo.), permits predicting siRNAs for any nucleic acid sequence, and is available on the World Wide Web at dharmacon.com. Programs for designing siRNAs are also available from others, including Genscript (available on the Web at genscript.com/ssl-bin/app/rnai) and, to academic and non-profit researchers, from the Whitehead Institute for Biomedical Research found on the worldwide web at “jura.wi.mit.edu/pubint/http://iona.wi.mit.edu/siRNAext/.”

For example, using the program available from the Whitehead Institute, the following sEH target sequences and siRNA sequences can be generated:

1) Target:
(SEQ ID NO: 3)
CAGTGTTCATTGGCCATGACTGG
Sense-siRNA:
(SEQ ID NO: 4)
5′-GUGUUCAUUGGCCAUGACUTT-3′
Antisense-siRNA:
(SEQ ID NO: 5)
5′-AGUCAUGGCCAAUGAACACTT-3′
2) Target:
(SEQ ID NO: 6)
GAAAGGCTATGGAGAGTCATCTG
Sense-siRNA:
(SEQ ID NO: 7)
5′-AAGGCUAUGGAGAGUCAUCTT-3′
Antisense-siRNA:
(SEQ ID NO: 8)
5′-GAUGACUCUCCAUAGCCUUTT-3′
3) Target
(SEQ ID NO: 9)
AAAGGCTATGGAGAGTCATCTGC
Sense-siRNA:
(SEQ ID NO: 10)
5′-AGGCUAUGGAGAGUCAUCUTT-3′
Antisense-siRNA:
(SEQ ID NO: 11)
5′-AGAUGACUCUCCAUAGCCUTT-3′
4) Target:
(SEQ ID NO: 12)
CAAGCAGTGTTCATTGGCCATGA
Sense-siRNA:
(SEQ ID NO: 13)
5′-AGCAGUGUUCAUUGGCCAUTT-3′
Antisense-siRNA:
(SEQ ID NO: 14)
5′-AUGGCCAAUGAACACUGCUTT-3′
5) Target:
(SEQ ID NO: 15)
CAGCACATGGAGGACTGGATTCC
Sense-siRNA:
(SEQ ID NO: 16)
5′-GCACAUGGAGGACUGGAUUTT-3′
Antisense-siRNA:
(SEQ ID NO: 17)
5′-AAUCCAGUCCUCCAUGUGCTT-3′

Alternatively, siRNA can be generated using kits which generate siRNA from the gene. For example, the “Dicer siRNA Generation” kit (catalog number T510001, Gene Therapy Systems, Inc., San Diego, Calif.) uses the recombinant human enzyme “dicer” in vitro to cleave long double stranded RNA into 22 bp siRNAs. By having a mixture of siRNAs, the kit permits a high degree of success in generating siRNAs that will reduce expression of the target gene. Similarly, the Silencer™ siRNA Cocktail Kit (RNase III) (catalog no. 1625, Ambion, Inc., Austin, Tex.) generates a mixture of siRNAs from dsRNA using RNase III instead of dicer. Like dicer, RNase III cleaves dsRNA into 12-30 bp dsRNA fragments with 2 to 3 nucleotide 3′ overhangs, and 5′-phosphate and 3′-hydroxyl termini. According to the manufacturer, dsRNA is produced using T7 RNA polymerase, and reaction and purification components included in the kit. The dsRNA is then digested by RNase III to create a population of siRNAs. The kit includes reagents to synthesize long dsRNAs by in vitro transcription and to digest those dsRNAs into siRNA-like molecules using RNase III. The manufacturer indicates that the user need only supply a DNA template with opposing T7 phage polymerase promoters or two separate templates with promoters on opposite ends of the region to be transcribed.

The siRNAs can also be expressed from vectors. Typically, such vectors are administered in conjunction with a second vector encoding the corresponding complementary strand. Once expressed, the two strands anneal to each other and form the functional double stranded siRNA. One exemplar vector suitable for use in the invention is pSuper, available from OligoEngine, Inc. (Seattle, Wash.). In some embodiments, the vector contains two promoters, one positioned downstream of the first and in antiparallel orientation. The first promoter is transcribed in one direction, and the second in the direction antiparallel to the first, resulting in expression of the complementary strands. In yet another set of embodiments, the promoter is followed by a first segment encoding the first strand, and a second segment encoding the second strand. The second strand is complementary to the palindrome of the first strand. Between the first and the second strands is a section of RNA serving as a linker (sometimes called a “spacer”) to permit the second strand to bend around and anneal to the first strand, in a configuration known as a “hairpin.”

The formation of hairpin RNAs, including use of linker sections, is well known in the art. Typically, an siRNA expression cassette is employed, using a Polymerase III promoter such as human U6, mouse U6, or human H1. The coding sequence is typically a 19-nucleotide sense siRNA sequence linked to its reverse complementary antisense siRNA sequence by a short spacer. Nine-nucleotide spacers are typical, although other spacers can be designed. For example, the Ambion website indicates that its scientists have had success with the spacer TTCAAGAGA (SEQ ID NO:18). Further, 5-6 T's are often added to the 3′ end of the oligonucleotide to serve as a termination site for Polymerase III. See also, Yu et al., Mol Ther 7(2):228-36 (2003); Matsukura et al., Nucleic Acids Res 31(15):e77 (2003).

As an example, the siRNA targets identified above can be targeted by hairpin siRNA as follows. To attack the same targets by short hairpin RNAs, produced by a vector (permanent RNAi effect), sense and antisense strand can be put in a row with a loop forming sequence in between and suitable sequences for an adequate expression vector to both ends of the sequence. The following are non-limiting examples of hairpin sequences that can be cloned into the pSuper vector:

1) Target:
(SEQ ID NO: 19)
CAGTGTTCATTGGCCATGACTGG
Sense strand:
(SEQ ID NO: 20)
5′-GATCCCCGTGTTCATTGGCCATGACTTTCAA
GAGAAGTCATGGCCAATGAACACTTTTT-3′
Antisense strand:
(SEQ ID NO: 21)
5′-AGCTAAAAAGTGTTCATTGGCCATGACTTCTCTT
GAAAGTCATGGCCAATGAACACGGG-3′
2) Target:
(SEQ ID NO: 22)
GAAAGGCTATGGAGAGTCATCTG
Sense strand:
(SEQ ID NO: 23)
5′-GATCCCCAAGGCTATGGAGAGTCATCTTCAAGAGAGA
TGACTCTCCATAGCCTTTTTTT-3′
Antisense strand:
(SEQ ID NO: 24)
5′-AGCTAAAAAAAGGCTATGGAGAGTCATCTCTCTTGAA
GATGACTCTCCATAGCCTTGGG-3′
3) Target:
(SEQ ID NO: 25)
AAAGGCTATGGAGAGTCATCTGC
Sense strand:
(SEQ ID NO: 26)
5′-GATCCCCAGGCTATGGAGAGTCATCTTTCAAGAGAAG
ATGACTCTCCATAGCCTTTTTT-3′
Antisense strand:
(SEQ ID NO: 27)
5′-AGCTAAAAAAGGCTATGGAGAGTCATCATCTCTTGAAAGATGACTCT
CCATAGCCTGGG-3′
4) Target:
(SEQ ID NO: 28)
CAAGCAGTGTTCATTGGCCATGA
Sense strand:
(SEQ ID NO: 29)
5′-GATCCCCAGCAGTGTTCATTGGCCATTTCAAGAGAATG
GCCAATGAACACTGCTTTTTT-3′
Antisense strand:
(SEQ ID NO: 30)
5′-AGCTAAAAAAGCAGTGTTCATTGGCCATTCTCTTGAAATG
GCCAATGAACACTGCTGGG-3′
5) Target:
(SEQ ID NO: 31)
CAGCACATGGAGGACTGGATTCC
Sense strand
(SEQ ID NO: 32)
5′-GATCCCCGCACATGGAGGACTGGATTTTCAAGAGAAATC
CAGTCCTCCATGTGCTTTTT-3′
Antisense strand:
(SEQ ID NO: 33)
5′-AGCTAAAAAGCACATGGAGGACTGGATTTCTCTTGAAAA
TCCAGTCCTCCATGTGCGGG-3′

In addition to siRNAs, other means are known in the art for inhibiting the expression of antisense molecules, ribozymes, and the like are well known to those of skill in the art. The nucleic acid molecule can be a DNA probe, a riboprobe, a peptide nucleic acid probe, a phosphorothioate probe, or a 2′-O methyl probe.

Generally, to assure specific hybridization, the antisense sequence is substantially complementary to the target sequence. In certain embodiments, the antisense sequence is exactly complementary to the target sequence. The antisense polynucleotides may also include, however, nucleotide substitutions, additions, deletions, transitions, transpositions, or modifications, or other nucleic acid sequences or non-nucleic acid moieties so long as specific binding to the relevant target sequence corresponding to the sEH gene is retained as a functional property of the polynucleotide. In one embodiment, the antisense molecules form a triple helix-containing, or “triplex” nucleic acid. Triple helix formation results in inhibition of gene expression by, for example, preventing transcription of the target gene (see, e.g., Cheng et al., 1988, J. Biol. Chem. 263:15110; Ferrin and Camerini-Otero, 1991, Science 354:1494; Ramdas et al., 1989, J. Biol. Chem. 264:17395; Strobel et al., 1991, Science 254:1639; and Rigas et al., 1986, Proc. Natl. Acad. Sci. U.S.A. 83:9591)

Antisense molecules can be designed by methods known in the art. For example, Integrated DNA Technologies (Coralville, Iowa) makes available a program found on the worldwide web “biotools.idtdna.com/antisense/AntiSense.aspx”, which will provide appropriate antisense sequences for nucleic acid sequences up to 10,000 nucleotides in length. Using this program with the sEH gene provides the following exemplar sequences:

1)
(SEQ ID NO: 34)
UGUCCAGUGCCCACAGUCCU
2)
(SEQ ID NO: 35)
UUCCCACCUGACACGACUCU
3)
(SEQ ID NO: 36)
GUUCAGCCUCAGCCACUCCU
4)
(SEQ ID NO: 37)
AGUCCUCCCGCUUCACAGA
5)
(SEQ ID NO: 38)
GCCCACUUCCAGUUCCUUUCC

In another embodiment, ribozymes can be designed to cleave the mRNA at a desired position. (See, e.g., Cech, 1995, Biotechnology 13:323; and Edgington, 1992, Biotechnology 10:256 and Hu et al., PCT Publication WO 94/03596).

The antisense nucleic acids (DNA, RNA, modified, analogues, and the like) can be made using any suitable method for producing a nucleic acid, such as the chemical synthesis and recombinant methods disclosed herein and known to one of skill in the art. In one embodiment, for example, antisense RNA molecules may be prepared by de novo chemical synthesis or by cloning. For example, an antisense RNA can be made by inserting (ligating) a sEH gene sequence in reverse orientation operably linked to a promoter in a vector (e.g., plasmid). Provided that the promoter and, preferably termination and polyadenylation signals, are properly positioned, the strand of the inserted sequence corresponding to the noncoding strand are transcribed and act as an antisense oligonucleotide.

It are appreciated that the oligonucleotides can be made using nonstandard bases (e.g., other than adenine, cytidine, guanine, thymine, and uridine) or nonstandard backbone structures to provides desirable properties (e.g., increased nuclease-resistance, tighter-binding, stability or a desired Tm). Techniques for rendering oligonucleotides nuclease-resistant include those described in PCT Publication WO 94/12633. A wide variety of useful modified oligonucleotides may be produced, including oligonucleotides having a peptide-nucleic acid (PNA) backbone (Nielsen et al., 1991, Science 254:1497) or incorporating 2′-O-methyl ribonucleotides, phosphorothioate nucleotides, methyl phosphonate nucleotides, phosphotriester nucleotides, phosphorothioate nucleotides, phosphoramidates.

Proteins have been described that have the ability to translocate desired nucleic acids across a cell membrane. Typically, such proteins have amphiphilic or hydrophobic subsequences that have the ability to act as membrane-translocating carriers. For example, homeodomain proteins have the ability to translocate across cell membranes. The shortest internalizable peptide of a homeodomain protein, Antennapedia, was found to be the third helix of the protein, from amino acid position 43 to 58 (see, e.g., Prochiantz, Current Opinion in Neurobiology 6:629-634 (1996). Another subsequence, the h (hydrophobic) domain of signal peptides, was found to have similar cell membrane translocation characteristics (see, e.g., Lin et al., J. Biol. Chem. 270:14255-14258 (1995)). Such subsequences can be used to translocate oligonucleotides across a cell membrane. Oligonucleotides can be conveniently derivatized with such sequences. For example, a linker can be used to link the oligonucleotides and the translocation sequence. Any suitable linker can be used, e.g., a peptide linker or any other suitable chemical linker.

More recently, it has been discovered that siRNAs can be introduced into mammals without eliciting an immune response by encapsulating them in nanoparticles of cyclodextrin. Information on this method can be found on the worldwide web at “nature.com/news/2005/050418/full/050418-6.html.”

In another method, the nucleic acid is introduced directly into superficial layers of the skin or into muscle cells by a jet of compressed gas or the like. Methods for administering naked polynucleotides are well known and are taught, for example, in U.S. Pat. No. 5,830,877 and International Publication Nos. WO 99/52483 and WO 94/21797. Devices for accelerating particles into body tissues using compressed gases are described in, for example, U.S. Pat. Nos. 6,592,545, 6,475,181, and 6,328,714. The nucleic acid may be lyophilized and may be complexed, for example, with polysaccharides to form a particle of appropriate size and mass for acceleration into tissue. Conveniently, the nucleic acid can be placed on a gold bead or other particle which provides suitable mass or other characteristics. Use of gold beads to carry nucleic acids into body tissues is taught in, for example, U.S. Pat. Nos. 4,945,050 and 6,194,389.

The nucleic acid can also be introduced into the body in a virus modified to serve as a vehicle without causing pathogenicity. The virus can be, for example, adenovirus, fowlpox virus or vaccinia virus.

miRNAs and siRNAs differ in several ways: miRNA derive from points in the genome different from previously recognized genes, while siRNAs derive from mRNA, viruses or transposons, miRNA derives from hairpin structures, while siRNA derives from longer duplexed RNA, miRNA is conserved among related organisms, while siRNA usually is not, and miRNA silences loci other than that from which it derives, while siRNA silences the loci from which it arises. Interestingly, miRNAs tend not to exhibit perfect complementarity to the mRNA whose expression they inhibit. See, McManus et al., supra. See also, Cheng et al., Nucleic Acids Res. 33(4):1290-7 (2005); Robins and Padgett, Proc Natl Acad Sci USA. 102(11):4006-9 (2005); Brennecke et al., PLoS Biol. 3(3):e85 (2005). Methods of designing miRNAs are known. See, e.g., Zeng et al., Methods Enzymol. 392:371-80 (2005); Krol et al., J Biol Chem. 279(40):42230-9 (2004); Ying and Lin, Biochem Biophys Res Commun. 326(3):515-20 (2005).

In some embodiments, the endogenous polynucleotide encoding sEH in the subject can be rendered non-functional or non-expressing, e.g., by employing gene therapy methodologies. This can be accomplished using any method known in the art, including the working embodiment described herein. In varying embodiments, the endogenous gene encoding sEH in the subject is rendered non-functional or non-expressing in certain desired tissues, e.g., in renal tissue or more specifically in podocyte cells, as demonstrated herein. In varying embodiments, the endogenous gene encoding sEH in the subject is rendered non-functional or non-expressing by employing homologous recombination, mutating, replacing or eliminating the functional or expressing gene encoding sEH. Illustrative methods are known in the art and described, e.g., in Flynn, et al., Exp Hematol. (2015) Jun. 19. pii: S0301-472X(15)00207-6 (using CRISPR); Truong, et al, Nucleic Acids Res. (2015) Jun. 16. pii: gkv601 (using split-Cas9); Yang, Mil Med Res. (2015) May 9; 2:11 (using CRISPR-Cas9); and Imai, et al., Intern Med. (2004) February; 43(2):85-96.

f. Epoxygenated Fatty Acids

In some embodiments, an epoxygenated fatty acid is administered as an agent that increases epoxygenated fatty acids. Illustrative epoxygenated fatty acids include epoxides of linoleic acid, eicosapentaenoic acid (“EPA”) and docosahexaenoic acid (“DHA”).

The fatty acids eicosapentaenoic acid (“EPA”) and docosahexaenoic acid (“DHA”) have recently become recognized as having beneficial effects, and fish oil tablets, which are a good source of these fatty acids, are widely sold as supplements. In 2003, it was reported that these fatty acids reduced pain and inflammation. Sethi, S. et al., Blood 100: 1340-1346 (2002). The paper did not identify the mechanism of action, nor the agents responsible for this relief.

Cytochrome P450 (“CYP450”) metabolism produces cis-epoxydocosapentaenoic acids (“EpDPEs”) and cis-epoxyeicosatetraenoic acids (“EpETEs”) from docosahexaenoic acid (“DHA”) and eicosapentaenoic acid (“EPA”), respectively. These epoxides are known endothelium-derived hyperpolarizing factors (“EDHFs”). These EDHFs, and others yet unidentified, are mediators released from vascular endothelial cells in response to acetylcholine and bradykinin, and are distinct from the NOS- (nitric oxide) and COX-derived (prostacyclin) vasodilators. Overall cytochrome P450 (CYP450) metabolism of polyunsaturated fatty acids produces epoxides, such as EETs, which are prime candidates for the active mediator(s). 14(15)-EpETE, for example, is derived via epoxidation of the 14,15-double bond of EPA and is the ω-3 homolog of 14(15)-EpETrE (“14(15)EET”) derived via epoxidation of the 14,15-double bond of arachidonic acid.

As mentioned, it is beneficial to elevate the levels of EETs, which are epoxides of the fatty acid arachidonic acid. Our studies of the effects of EETs has led us to realization that the anti-inflammatory effect of EPA and DHA are likely due to increasing the levels of the epoxides of these two fatty acids. Thus, increasing the levels of epoxides of EPA, of DHA, or of both, will act to reduce pain and inflammation, and symptoms associated with diabetes and metabolic syndromes, in mammals in need thereof. This beneficial effect of the epoxides of these fatty acids has not been previously recognized. Moreover, these epoxides have not previously been administered as agents, in part because, as noted above, epoxides have generally been considered too labile to be administered.

Like EETs, the epoxides of EPA and DHA are substrates for sEH. The epoxides of EPA and DHA are produced in the body at low levels by the action of cytochrome P450s. Endogenous levels of these epoxides can be maintained or increased by the administration of sEHI. However, the endogeous production of these epoxides is low and usually occurs in relatively special circumstances, such as the resolution of inflammation. Our expectation is that administering these epoxides from exogenous sources will aid in the resolution of inflammation and in reducing pain, as well as with symptoms of diabetes and metabolic syndromes. It is further beneficial with pain or inflammation to inhibit sEH with sEHI to reduce hydrolysis of these epoxides, thereby maintaining them at relatively high levels.

EPA has five unsaturated bonds, and thus five positions at which epoxides can be formed, while DHA has six. The epoxides of EPA are typically abbreviated and referred to generically as “EpETEs”, while the epoxides of DHA are typically abbreviated and referred to generically as “EpDPEs”. The specific regioisomers of the epoxides of each fatty acid are set forth in the following Table 3:

TABLE 3
Regioisomers of Eicosapentaenoic acid (“EPA”) epoxides:
1. Formal name: (±)5(6)-epoxy-8Z,11Z,14Z,17Z-eicosatetraenoic acid,
Synonym 5(6)-epoxy Eicosatetraenoic acid
Abbreviation 5(6)-EpETE
2. Formal name: (±)8(9)-epoxy-5Z,11Z,14Z,17Z-eicosatetraenoic acid,
Synonym 8(9)-epoxy Eicosatetraenoic acid
Abbreviation 8(9)-EpETE
3. Formal name: (±)11(12)-epoxy-5Z,8Z,14Z,17Z-eicosatetraenoic acid,
Synonym 11(12)-epoxy Eicosatetraenoic acid
Abbreviation 11(12)-EpETE
4. Formal name: (±)14(15)-epoxy-5Z,8Z,11Z,17Z-eicosatetraenoic acid,
Synonym 14(15)-epoxy Eicosatetraenoic acid
Abbreviation 14(15)-EpETE
5. Formal name: (±)17(18)-epoxy-5Z,8Z,11Z,14Z-eicosatetraenoic acid,
Synonym 17(18)-epoxy Eicosatetraenoic acid
Abbreviation 17(18)-EpETE
Regioisomers of Docosahexaenoic acid (“DHA”) epoxides:
1. Formal name: (±) 4(5)-epoxy-7Z,10Z,13Z,16Z,19Z-docosapentaenoic
acid,
Synonym 4(5)-epoxy Docosapentaenoic acid
Abbreviation 4(5)-EpDPE
2. Formal name: (±) 7(8)-epoxy-4Z,10Z,13Z,16Z,19Z-docosapentaenoic
acid,
Synonym 7(8)-epoxy Docosapentaenoic acid
Abbreviation 7(8)-EpDPE
3. Formal name: (±)10(11)-epoxy-4Z,7Z,13Z,16Z,19Z-docosapentaenoic
acid,
Synonym 10(11)-epoxy Docosapentaenoic acid
Abbreviation 10(11)-EpDPE
4. Formal name: (±)13(14)-epoxy-4Z,7Z,10Z,16Z,19Z-docosapentaenoic
acid,
Synonym 13(14)-epoxy Docosapentaenoic acid
Abbreviation 13(14)-EpDPE
5. Formal name: (±) 16(17)-epoxy-4Z,7Z,10Z,13Z,19Z-docosapentaenoic
acid,
Synonym 16(17)-epoxy Docosapentaenoic acid
Abbreviation 16(17)-EpDPE
6. Formal name: (±) 19(20)-epoxy-4Z,7Z,10Z,13Z,16Z-docosapentaenoic
acid,
Synonym 19(20)-epoxy Docosapentaenoic acid
Abbreviation 19(20)-EpDPE

Any of these epoxides, or combinations of any of these, can be administered in the compositions and methods.

5. Formulation and Administration

In various embodiments of the compositions, the agent that increases epoxygenated fatty acids (e.g., an inhibitor of sEH, an EET, an epoxygenated fatty acid, and mixtures thereof) is co-administered with the agent that reduces and/or inhibits ER stress (e.g., PBA). In some embodiments, the agent that increases epoxygenated fatty acids comprises an epoxide of EPA, an epoxide of DHA, or epoxides of both, and an sEHI.

The agent that increases epoxygenated fatty acids and the agent that inhibits and/or reduces ER stress independently can be prepared and administered in a wide variety of oral, parenteral and topical dosage forms. The agent that increases epoxygenated fatty acids and the agent that inhibits and/or reduces ER stress can be administered via the same or different routes of administration. In varying embodiments, the agent that increases epoxygenated fatty acids and the agent that inhibits and/or reduces ER stress independently can be administered orally, by injection, that is, intravenously, intramuscularly, intracutaneously, subcutaneously, intraduodenally, or intraperitoneally. The agent that increases epoxygenated fatty acids and the agent that inhibits and/or reduces ER stress can also be administered by inhalation, for example, intranasally. Additionally, the agent that increases epoxygenated fatty acids and the agent that inhibits and/or reduces ER stress can be administered transdermally.

In varying embodiments, one or both of the agent that increases epoxygenated fatty acids (e.g., an sEHI or a pharmaceutically acceptable salt of the inhibitor and, optionally, one or more EETs or epoxides of EPA or of DHA, or of both), and/or the agent that reduces and/or inhibits ER stress are specifically, predominantly or preferentially targeted to the kidneys. Methods for preferentially targeting therapeutic agents to renal tissues are known in the art and find use. Illustrative methods are described, e.g., Zuckerman, et al., Adv Chronic Kidney Dis. (2013) 20(6):500-7; Wang, et al., Int J Pharm. (2013) 456(1):223-34; Lin, et al., J Control Release. (2013) 167(2):148-56; Geng, et al., Bioconjug Chem. (2012) 23(6):1200-10; Dolman, et al., Int J Nanomedicine. (2012) 7:417-33; Tomita, et al., J Gene Med. (2002) 4(5):527-35; and Zhou, et al., Acta Pharmaceutica Sinica B (2014) 4(1):37-42.

Furthermore, the agent that increases epoxygenated fatty acids and the agent that inhibits and/or reduces ER stress can be co-formulated in a single composition or can be formulated for separate co-administration. Accordingly, in some embodiments, the methods contemplate administration of compositions comprising a pharmaceutically acceptable carrier or excipient, an agent that increases epoxygenated fatty acids (e.g., an sEHI or a pharmaceutically acceptable salt of the inhibitor and, optionally, one or more EETs or epoxides of EPA or of DHA, or of both), and optionally an agent that reduces and/or inhibits ER stress. In some embodiments, the methods comprise administration of an sEHI and one or more epoxides of EPA or of DHA, or of both.

For preparing the pharmaceutical compositions, the pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component. In tablets, the active component is mixed with the carrier having the necessary binding properties in suitable proportions and compacted in the shape and size desired. The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution. Transdermal administration can be performed using suitable carriers. If desired, apparatuses designed to facilitate transdermal delivery can be employed. Suitable carriers and apparatuses are well known in the art, as exemplified by U.S. Pat. Nos. 6,635,274, 6,623,457, 6,562,004, and 6,274,166.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active components in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

A variety of solid, semisolid and liquid vehicles have been known in the art for years for topical application of agents to the skin. Such vehicles include creams, lotions, gels, balms, oils, ointments and sprays. See, e.g., Provost C. “Transparent oil-water gels: a review,” Int J Cosmet Sci. 8:233-247 (1986), Katz and Poulsen, Concepts in biochemical pharmacology, part I. In: Brodie B B, Gilette J R, eds. Handbook of Experimental Pharmacology. Vol. 28. New York, N.Y.: Springer; 107-174 (1971), and Hadgcraft, “Recent progress in the formulation of vehicles for topical applications,” Br J Dermatol., 81:386-389 (1972). A number of topical formulations of analgesics, including capsaicin (e.g., Capsin®), so-called “counter-irritants” (e.g., Icy-Hot®, substances such as menthol, oil of wintergreen, camphor, or eucalyptus oil compounds which, when applied to skin over an area presumably alter or off-set pain in joints or muscles served by the same nerves) and salicylates (e.g. BenGay®), are known and can be readily adapted for topical administration of sEHI by replacing the active ingredient or ingredient with an sEHI, with or without EETs. It is presumed that the person of skill is familiar with these various vehicles and preparations and they need not be described in detail herein.

The agent that increases epoxygenated fatty acids (e.g., an inhibitor of sEH, an EET, an epoxygenated fatty acid, and mixtures thereof), optionally mixed with an anti-inflammatory and/or analgesic agent, can be mixed into such modalities (creams, lotions, gels, etc.) for topical administration. In general, the concentration of the agents provides a gradient which drives the agent into the skin. Standard ways of determining flux of drugs into the skin, as well as for modifying agents to speed or slow their delivery into the skin are well known in the art and taught, for example, in Osborne and Amann, eds., Topical Drug Delivery Formulations, Marcel Dekker, 1989. The use of dermal drug delivery agents in particular is taught in, for example, Ghosh et al., eds., Transdermal and Topical Drug Delivery Systems, CRC Press, (Boca Raton, Fla., 1997).

In some embodiments, the agents are in a cream. Typically, the cream comprises one or more hydrophobic lipids, with other agents to improve the “feel” of the cream or to provide other useful characteristics. In one embodiment, for example, a cream may contain 0.01 mg to 10 mg of sEHI, with or without one or more EETs, per gram of cream in a white to off-white, opaque cream base of purified water USP, white petrolatum USP, stearyl alcohol NF, propylene glycol USP, polysorbate 60 NF, cetyl alcohol NF, and benzoic acid USP 0.2% as a preservative. In various embodiments, an agent that increases epoxygenated fatty acids (e.g., an sEHI or a pharmaceutically acceptable salt of the inhibitor and, optionally, one or more EETs or epoxides of EPA or of DHA, or of both), and/or an agent that reduces and/or inhibits ER stress can be mixed into a commercially available cream, Vanicream® (Pharmaceutical Specialties, Inc., Rochester, Minn.) comprising purified water, white petrolatum, cetearyl alcohol and ceteareth-20, sorbitol solution, propylene glycol, simethicone, glyceryl monostearate, polyethylene glycol monostearate, sorbic acid and BHT.

In other embodiments, the agent or agents are in a lotion. Typical lotions comprise, for example, water, mineral oil, petrolatum, sorbitol solution, stearic acid, lanolin, lanolin alcohol, cetyl alcohol, glyceryl stearate/PEG-100 stearate, triethanolamine, dimethicone, propylene glycol, microcrystalline wax, tri (PPG-3 myristyl ether) citrate, disodium EDTA, methylparaben, ethylparaben, propylparaben, xanthan gum, butylparaben, and methyldibromo glutaronitrile.

In some embodiments, the agent is, or agents are, in an oil, such as jojoba oil. In some embodiments, the agent is, or agents are, in an ointment, which may, for example, white petrolatum, hydrophilic petrolatum, anhydrous lanolin, hydrous lanolin, or polyethylene glycol. In some embodiments, the agent is, or agents are, in a spray, which typically comprise an alcohol and a propellant. If absorption through the skin needs to be enhanced, the spray may optionally contain, for example, isopropyl myristate.

Whatever the form in which the agents that inhibit sEH are topically administered (that is, whether by solid, liquid, lotion, gel, spray, etc.), in various embodiments they are administered at a dosage of about 0.01 mg to 10 mg per 10 cm². An exemplary dose for systemic administration of an inhibitor of sEH is from about 0.001 μg/kg to about 100 mg/kg body weight of the mammal. In various embodiments, dose and frequency of administration of an sEH inhibitor are selected to produce plasma concentrations within the range of 2.5 μM and 30 nM.

The agent that increases epoxygenated fatty acids (e.g., an inhibitor of sEH, an EET, an epoxygenated fatty acid, and mixtures thereof), optionally mixed with an anti-inflammatory and/or analgesic agent, can be introduced into the bowel by use of a suppository. As is known in the art, suppositories are solid compositions of various sizes and shapes intended for introduction into body cavities. Typically, the suppository comprises a medication, which is released into the immediate area from the suppository. Typically, suppositories are made using a fatty base, such as cocoa butter, that melts at body temperature, or a water-soluble or miscible base, such as glycerinated gelatin or polyethylene glycol.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The term “unit dosage form”, as used in the specification, refers to physically discrete units suitable as unitary dosages for human subjects and animals, each unit containing a predetermined quantity of active material calculated to produce the desired pharmaceutical effect in association with the required pharmaceutical diluent, carrier or vehicle. The specifications for the novel unit dosage forms of this invention are dictated by and directly dependent on (a) the unique characteristics of the active material and the particular effect to be achieved and (b) the limitations inherent in the art of compounding such an active material for use in humans and animals, as disclosed in detail in this specification.

A therapeutically effective amount or a sub-therapeutic amount of the agent that increases epoxygenated fatty acids can be co-administered with the agent that reduces and/or inhibits ER stress (e.g., PBA). The dosage of the specific compounds depends on many factors that are well known to those skilled in the art. They include for example, the route of administration and the potency of the particular compound. An exemplary dose is from about 0.001 μg/kg to about 100 mg/kg body weight of the mammal. Determination of an effective amount is well within the capability of those skilled in the art, especially in light of the detailed disclosure provided herein. Generally, an efficacious or effective amount of a combination of one or more polypeptides of the present invention is determined by first administering a low dose or small amount of a polypeptide or composition and then incrementally increasing the administered dose or dosages, adding a second or third medication as needed, until a desired effect of is observed in the treated subject with minimal or no toxic side effects. Applicable methods for determining an appropriate dose and dosing schedule for administration of a combination of the present invention are described, for example, in Goodman and Gilman's The Pharmacological Basis of Therapeutics, 12th Edition, 2010, McGraw-Hill Professional; in a Physicians' Desk Reference (PDR), 69^{th} Edition, 2015, PDR Network; in Remington: The Science and Practice of Pharmacy, 21^{st} Ed., 2005, supra; and in Martindale: The Complete Drug Reference, Sweetman, 2005, London: Pharmaceutical Press., and in Martindale, Martindale: The Extra Pharmacopoeia, 31st Edition., 1996, Amer Pharmaceutical Assn, each of which are hereby incorporated herein by reference.

EETs, EpDPEs, or EpETEs are unstable, and can be converted to the corresponding diols, in acidic conditions, such as those in the stomach. To avoid this, EETs, EpDPEs, or EpETEs can be administered intravenously or by injection. EETs, EpDPEs, or EpETEs intended for oral administration can be encapsulated in a coating that protects the compounds during passage through the stomach. For example, the EETs, EpDPEs, or EpETEs can be provided with a so-called “enteric” coating, such as those used for some brands of aspirin, or embedded in a formulation. Such enteric coatings and formulations are well known in the art. In some formulations, the compositions are embedded in a slow-release formulation to facilitate administration of the agents over time.

It is understood that, like all drugs, sEHIs have half-lives defined by the rate at which they are metabolized by or excreted from the body, and that the sEHIs will have a period following administration during which they are present in amounts sufficient to be effective. If EETs, EpDPEs, or EpETEs are administered after the sEHI is administered, therefore, it is desirable that the EETs, EpDPEs, or EpETEs be administered during the period during which the sEHI are present in amounts to be effective in delaying hydrolysis of the EETs, EpDPEs, or EpETEs. Typically, the EETs, EpDPEs, or EpETEs are administered within 48 hours of administering an sEH inhibitor. Preferably, the EETs, EpDPEs, or EpETEs are administered within 24 hours of the sEHI, and even more preferably within 12 hours. In increasing order of desirability, the EETs, EpDPEs, or EpETEs are administered within 10, 8, 6, 4, 2, hours, 1 hour, or one half hour after administration of the inhibitor. When co-administered, the EETs, EpDPEs, or EpETEs are preferably administered concurrently with the sEHI.

6. Methods of Monitoring

Clinical efficacy can be monitored using any method known in the art. Measurable parameters to monitor efficacy will depend on the condition being treated. For monitoring the status or improvement of one or more symptoms associated with nephropathy and/or diabetes, both subjective parameters (e.g., patient reporting) and objective parameters (e.g., urine protein and/or glucose levels, blood urea nitrogen (BUN) levels, plasma glucose levels (random, fasting, or upon glucose challenge); blood hemoglobin A1c (HbA1c or A1c) levels; glycosylated hemoglobin (GHb) levels; microalbumin levels or albumin-to-creatinine ratio; insulin levels; C-peptide levels). Applicable assays for the monitoring of nephropathy and diabetes are known in the art. Behavioral changes in the subject (e.g., appetite, the ability to eat solid foods, grooming, sociability, energy levels, increased activity levels, weight gain, exhibition of increased comfort) are also relevant to all diseases and disease conditions associated with and/or caused at least in part by ER stress. These parameters can be measured using any methods known in the art. In varying embodiments, the different parameters can be assigned a score. Further, the scores of two or more parameters can be combined to provide an index for the subject.

Observation of the stabilization, improvement and/or reversal of one or more symptoms or parameters by a measurable amount indicates that the treatment or prevention regime is efficacious. Observation of the progression, increase or exacerbation of one or more symptoms indicates that the treatment or prevention regime is not efficacious. For example, in the case of diabetes, observation the improvement of one or both of subjective parameters (e.g., patient reporting) and objective parameters (e.g., urine protein and/or glucose levels, blood urea nitrogen (BUN) levels, plasma glucose levels (random, fasting, or upon glucose challenge); blood hemoglobin A1c (HbA1c or A1c) levels; glycosylated hemoglobin (GHb) levels; microalbumin levels or albumin-to-creatinine ratio; insulin levels; C-peptide levels) and/or behavioral changes in the subject (e.g., increased appetite, the ability to eat solid foods, improved/increased grooming, improved/increased sociability, increased energy levels, improved/increased activity levels, weight gain and/or stabilization, exhibition of increased comfort) after one or more co-administrations of the agent that reduces and/or inhibits ER stress (e.g., PBA) with an agent that increases epoxygenated fatty acids (e.g., an inhibitor of sEH) indicates that the treatment or prevention regime is efficacious. In the case of nephropathy, observation of the improvement of renal or kidney function (e.g., changes in urinary and/or blood markers), and/or behavioral changes in the subject (e.g., increased appetite, the ability to eat solid foods, improved/increased grooming, improved/increased sociability, increased energy levels, improved/increased activity levels, weight gain and/or stabilization, exhibition of increased comfort) after one or more co-administrations of the agent that reduces and/or inhibits ER stress (e.g., PBA) with an agent that increases epoxygenated fatty acids (e.g., an inhibitor of sEH) indicates that the treatment or prevention regime is efficacious. Likewise, observation of reduction or decline, lack of improvement or worsending of one or both of subjective parameters (e.g., patient reporting) and objective parameters (e.g., urine protein and/or glucose levels, blood urea nitrogen (BUN) levels, plasma glucose levels (random, fasting, or upon glucose challenge); blood hemoglobin A1c (HbA1c or A1c) levels; glycosylated hemoglobin (GHb) levels; microalbumin levels or albumin-to-creatinine ratio; insulin levels; C-peptide levels) related to renal or kidney function (e.g., changes in urinary and/or blood markers), and/or behavioral changes in the subject (e.g., decreased appetite, the inability to eat solid foods, decreased grooming, decreased sociability, decreased energy levels, decreased activity levels, weight loss, exhibition of increased discomfort) after one or more co-administrations of the agent that reduces and/or inhibits ER stress (e.g., PBA) with an agent that increases epoxygenated fatty acids (e.g., an inhibitor of sEH) indicates that the treatment or prevention regime is not efficacious.

In certain embodiments, the monitoring methods can entail determining a baseline value of a measurable biomarker or disease parameter in a subject before administering a dosage of the one or more active agents described herein, and comparing this with a value for the same measurable biomarker or parameter after a course of treatment.

In other methods, a control value (i.e., a mean and standard deviation) of the measurable biomarker or parameter is determined for a control population. In certain embodiments, the individuals in the control population have not received prior treatment and do not have the disease condition subject to treatment (e.g., nephropathy, pre-diabetes, diabetes and/or another disease condition associated with or caused at least in part by ER stress), nor are at risk of developing the disease condition subject to treatment (e.g., nephropathy, pre-diabetes, diabetes and/or another disease condition associated with or caused at least in part by ER stress). In such cases, if the value of the measurable biomarker or clinical parameter approaches the control value, then treatment is considered efficacious. In other embodiments, the individuals in the control population have not received prior treatment and have been diagnosed with the disease condition subject to treatment (e.g., nephropathy, pre-diabetes, diabetes, and/or another disease condition associated with or caused at least in part by ER stress). In such cases, if the value of the measurable biomarker or clinical parameter approaches the control value, then treatment is considered inefficacious.

In other methods, a subject who is not presently receiving treatment but has undergone a previous course of treatment is monitored for one or more of the biomarkers or clinical parameters to determine whether a resumption of treatment is required. The measured value of one or more of the biomarkers or clinical parameters in the subject can be compared with a value previously achieved in the subject after a previous course of treatment. Alternatively, the value measured in the subject can be compared with a control value (mean plus standard deviation) determined in population of subjects after undergoing a course of treatment. Alternatively, the measured value in the subject can be compared with a control value in populations of prophylactically treated subjects who remain free of symptoms of disease, or populations of therapeutically treated subjects who show amelioration of disease characteristics. In such cases, if the value of the measurable biomarker or clinical parameter approaches the control value, then treatment is considered efficacious and need not be resumed. In all of these cases, a significant difference relative to the control level (i.e., more than a standard deviation) is an indicator that treatment should be resumed in the subject.

7. Kits

Further provided are kits. In varying embodiments, the kits comprise one or more agents that increase the production and/or level of epoxygenated fatty acids and one or more inhibitors of endoplasmic reticular stress. Embodiments of the agents that increase the production and/or level of epoxygenated fatty acids and embodiments of inhibitors of endoplasmic reticular stress are as described above and herein. Embodiments of formulations of the agents are as described above and herein. In varying embodiments, the agent that increases the production and/or level of epoxygenated fatty acids and the inhibitor of endoplasmic reticular stress can be co-formulated for administration as a single composition. In some embodiments, the agent that increases the production and/or level of epoxygenated fatty acids and the inhibitor of endoplasmic reticular stress are formulated for separate administration, e.g., via the same or different route of administration. In varying embodiments, one or both the agent that increases the production and/or level of epoxygenated fatty acids and the inhibitor of endoplasmic reticular stress are provided in unitary dosages in the kits.

### EXAMPLES

The following examples are offered to illustrate, but not to limit the claimed invention.

### Example 1

### Soluble Epoxide Hydrolase in the Glomerular Podocyte is a Significant Contributor to Kidney Function

### Materials

Mouse Studies.

sEH-floxed (sEH^{fl/fl}) mice were backcrossed on C57Bl/6J background five times and were generated and kindly provided by Dr. D. Zeldin laboratory (NIEHS). Transgenic mice expressing Cre recombinase under the control of podocin promoter on C57Bl/6J background were purchased from Jackson laboratories. sEH^{fl/fl} mice were bred to podocin-Cre to generate mice lacking sEH in podocytes as described [23]. Genotyping for the sEH foxed allele and for the presence of Cre was performed by polymerase chain reaction (PCR), using DNA extracted from tails. Mice were maintained on a 12-hour light-dark cycle with free access to water and food. Mice were fed standard lab chow (Purina lab chow, #5001). For straptozotocin (STZ)-induced hyperglycemia studies 8-12 weeks old pod-sEHKO and control male mice received a single intraperitoneal injection of STZ (Sigma-Aldrich) (160 μg/g body weight) in 50 mM sodium citrate buffer as described [22, 24]. Metabolic studies were performed as detailed later and mice sacrificed 24 weeks after STZ injection. Kidneys were harvested and processed for biochemical and histological analyses. All mouse studies were conducted in line with federal regulations and were approved by the Institutional Animal Care and Use Committee at University of California Davis.

Metabolic Measurements.

Metabolic variables were determined in serum and urine samples from fed and fasted animals. Fed measurements were taken between 7-9 am and fasted measurements were done on mice fasted for at least 12 hours. Serum and urine albumin and creatinine concentrations were measured using corresponding kits (Sigma) according to manufacturer's instructions. Serum glucose was measured in blood using a glucometer (Home Aide Diagnostics) and in urine using Thermo Scientific™ infinity Glucose Hexokinase kit (Thermo Fisher Scientific). HDL-cholesterol concentrations were measured by an enzymatic colorimetric method using a commercial kit (Wako Pure Chemical Industries). For insulin tolerance tests (ITTs), mice were fasted for 4 h and injected intraperitoneally with 0.75 U/kg body weight human insulin (HumulinR; Eli Lilly). Blood glucose values were measured before and at 15, 30, 45, 60, 90 and 120 min post-injection. For glucose tolerance tests (GTTs), overnight-fasted mice were injected with 20% D-glucose at 2 mg/g body weight, and glucose was measured before and at 30, 60, 90 and 120 min following injection. Blood pressure was determined using the tail cuff method. Briefly, systolic and diastolic blood pressure was measured using the noninvasive blood pressure monitor (Columbus Instruments) by the tail cuff method wherein the average of three days blood pressure was used.

Histology and Electron Microscopy.

Kidney sections were fixed in 4% paraformaldehyde, embedded in paraffin, and deparaffinized in xylene, and then 4 μm sections were stained with hematoxylin-eosin, and periodic acid Schiff (PAS) using commercially available kits (Sigma) according to manufacturer’ recommendations. Transmission electron microscopy was carried on kidney cortical tissue from two mice per group. Kidneys were cut into two pieces on ice, fixed with 2.5% glutaraldehyde dissolved in 0.1 M sodium cacodylate (pH 7.4) at 4° C. overnight and washed in the same buffer. Tissue fragments were postfixed in 1% cacodylate-buffered OsO₄ for 2 h, dehydrated, and embedded in Epon. Ultrathin sections were stained with uranyl acetate and lead citrate and examined by transmission electron microscopy.

Podocyte Isolation.

Podocytes were isolated from control and pod-sEHKO mice using established protocols with modifications. Podocytes were isolated using a successive sieving approach using 3 screens with pore sizes of 250, 100, and 71 μm. Under aseptic conditions, kidneys from three animals were decapsulated and minced with a razor blade in Krebs-Henseleit saline solution (KHS) (119 mM NaCl, 4.7 mM KCl, 1.9 mM CaCl₂, 1.2 mM KH2PO4, 1.2 mM MgSO₄.7H₂O, and 25 mM NaHCO3, pH 7.4). Samples were pooled and pelleted at 500 g for 10 min then washed twice with KHS buffer. Prior the second wash, samples were passed through a 250 μm sieve and pelleted again at 500 g for 10 min then digested for 30 min at 37° C. in Hanks buffer containing collagenase D (0.1%), trypsin (0.25%), and DNase I (0.01%) in Hanks buffer. Solutions were then sieved through 100 μm sieve placed on the top of a 53-μm sieve. Podocytes were centrifuged for 5 min at 1500 g, 4° C. and resuspended in RPMI 1640 medium.

Cell Culture.

Murine kidney podocyte cell line E11 was purchased from Cell Lines Service (Eppelheim, Germany). These cells proliferate at a “permissive” temperature (33° C.). After transfer to “nonpermissive” temperature (37° C.), they enter growth arrest and express markers of differentiated in vivo podocytes [25]. This is important as in vivo podocytes are terminally differentiated cells and the immortalized podocytes express cellular markers and morphologically resemble differentiated podocytes. E11 cells were cultured at 33° C. in in RPMI medium supplemented with 2 mM L-glutamine and 10% fetal bovine serum. To induce differentiation, podocytes were grown at 37° C. for 14 days. Cells were then switched to high glucose (25 mM) or low glucose (5.6 mM) medium RPMI medium supplemented with 2 mM L-glutamine and 10% fetal bovine serum for 72 h. Twelve hours prior harvesting cells were treated with sEH inhibitor (1-trifluoromethoxyphenyl-3-(1-propionylpiperidin-4-yl) urea TPPU; 10 uM), ER stress inhibitor sodium 4-phenylbutyrate (4-PBA; 250 uM) and autophagy inhibitor (DBeQ:15 uM).

Biochemical Analyses.

Tissues were ground in liquid nitrogen and lysed using RIPA buffer. Lysates were clarified by centrifugation at 13,000 rpm for 10 min and protein concentrations were determined using bicinchoninic acid protein assay kit (Pierce Chemical). Proteins were resolved by SDS-PAGE and transferred to PVDF membranes. Immunoblotting of lysates was performed with antibodies for sEH that were developed in the Hammock laboratory [11], pIKKα/β (Ser178/180), IKKα/β, pIκBα (Ser32), IκBα, pNF-κBp65 (Ser536), NF-κBp65 and NF-κBp50, pERK1/2 (Tyr202/Thr204), pp38 (Thr180/Tyr182), p38, pJNK (Thr183/Tyr185), JNK, (all from Cell Signaling Technology; Danvers, Mass.) and cleaved Caspases 8, 9 and 3, pAMPK(Thr172), AMPK, PGC1α, ERK1/2, pPERK (Thr980), peIF2α (Ser51), eIF2α, sXBP1, IRE1α, Beclin, LC3, pSmad2(ser465), Smad2, TGFβRII, MCP1 and Tubulin (all from Santa Cruz Biotechnology). Antibodies for pIRE1α (Ser⁷²⁴) was purchased from Abcam (Cambridge, Mass.). Proteins were visualized using enhanced chemiluminescence (ECL, Amersham Biosciences) and pixel intensities of immuno-reactive bands were quantified using FluorChem 8900 (Alpha Innotech). Data for phosphorylated proteins are presented as phosphorylation level normalized to protein expression.

Total RNA was extracted from kidney and liver samples using TRIzol reagent (Invitrogen). cDNA was generated using high-capacity cDNA synthesis Kit (Applied Biosystems). mRNA levels were assessed by SYBR Green quantitative real time PCR using SsoAdvanced™ Universal SYBR® Green Supermix (iCycler, BioRad). Relative gene expression was quantitated using the ΔCT method with appropriate primers (Table 4) and normalized to Tata-box binding protein (Tbp). Briefly, the threshold cycle (Ct) was determined and relative gene expression was calculated as follows: fold change=2−Δ(ΔCt), where ΔCt=Ct target gene−Ct TBP (cycle difference) and Δ(ΔCt)=Ct (treated mice)−/Ct (control mice).

Statistical Analyses.

Data are expressed as means+standard error of the mean (SEM). Statistical analyses were performed using the IMP program (SAS Institute). ITTs, GTTs, body weight and adiposity data were analyzed by analysis of variance (ANOVA). Post-hoc analysis was performed using Tukey-Kramer honestly significant difference test. For biochemistry studies, comparisons between groups were performed using unpaired two-tailed Student's t test. Differences were considered significant at p<0.05 and highly significant at p<0.01.

### Results

Generation of Podocyte-Specific sEH Knockout Mice.

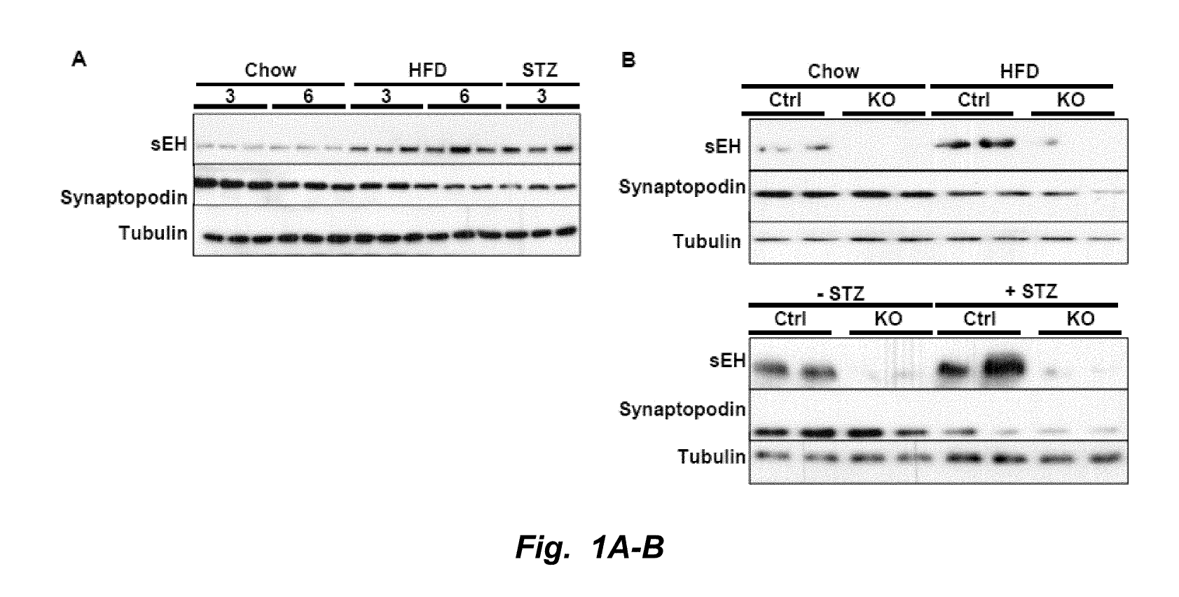

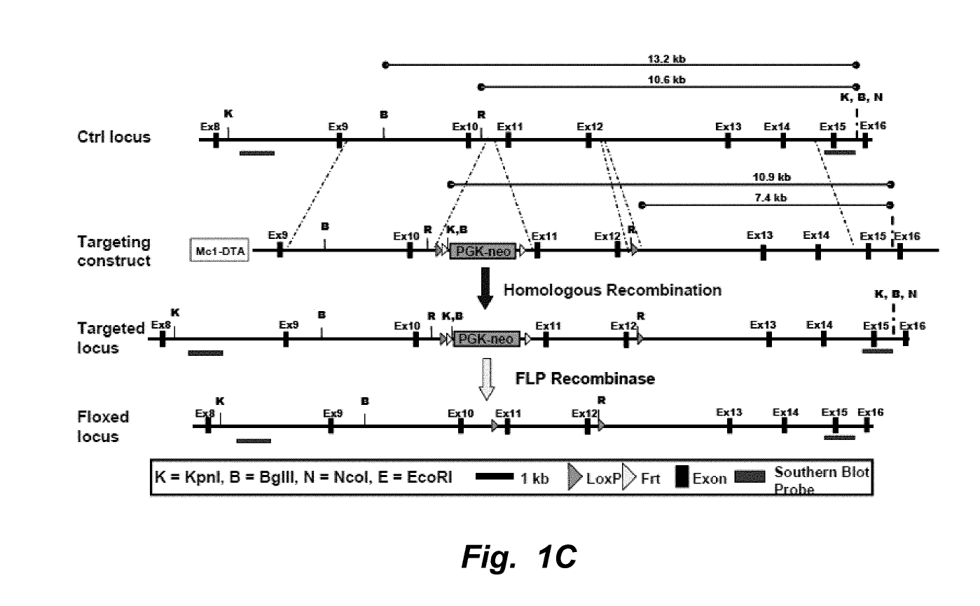

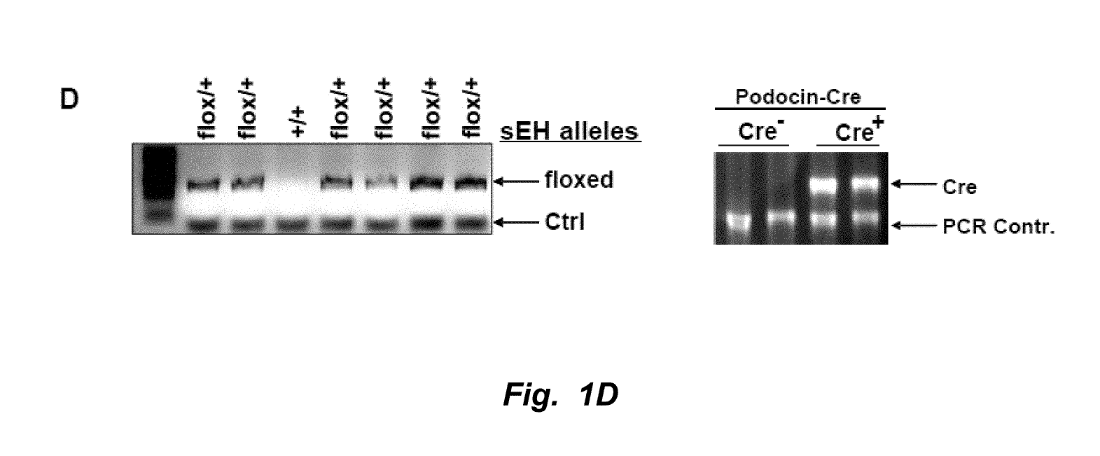

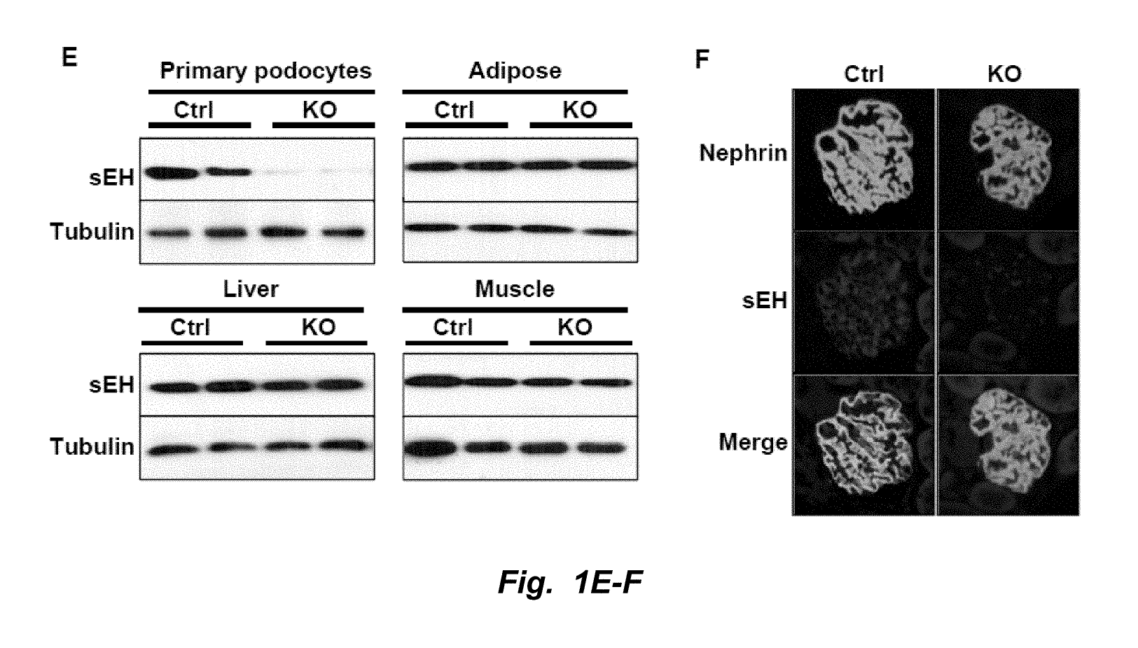

Cell type-specific expression of sEH in the kidney remains unclear. In total kidney lysates of diabetic mice some report attenuation of sEH protein expression [26] while others indicate increased expression [27]. We investigated sEH expression in podocytes and determined if it is modulated under high fat feeding and hyperglycemic conditions. Immunoblot analyses of total kidney lysates from wild type mice fed regular chow and HFD (3 and 6 months) and straptozotocin-treated mice revealed increased sEH protein expression in kidney upon high fat feeding and STZ treatment (FIG. 1A). In addition, primary podocytes were isolated from mice fed regular chow and HFD and those treated with STZ, then immunoblotted for sEH. To ensure that sEH antibodies were specific podocyte lysates from podocyte-specific sEH KO mice (see later) were included. In line with findings using total kidney lysates, sEH expression increased in isolated podocytes from HFD fed and STZ-treated mice compared with controls (FIG. 1B). Importantly, sEH expression was not observed in podocyte lysates from sEH knockout mice.

Regulated Expression of sEH in Podocytes Suggests that Dysregulation of sEH Signaling May be Relevant to Renal Function.

To determine the role of sEH in podocyte function we generated mice with podocyte sEH deletion by crossing sEH^{fl/fl} mice (FIG. 1C, D) to transgenic mice expressing Cre recombinase under the control of podocin promoter as described [23]. Podocyte sEH knockout (hereafter termed pod-sEHKO) mice survived to adulthood and did not display gross defects in the kidneys. Immunoblot analyses of isolated primary podocytes from control and pod-sEHKO mice revealed ablation of sEH expression in knockout mice compared with controls (FIG. 1E). In addition, sEH protein expression was comparable in different tissues (adipose, liver and muscle) suggesting specificity of deletion. Consistent with immunoblotting data, co-immunostaining of sEH and nephrin in kidney sections of control and pod-sEHKO mice demonstrated significant reduction of sEH in knockout mice confirming its ablation in podocytes (FIG. 1F). Collectively, these data demonstrate efficient and specific deletion of sEH in podocytes of pod-sEHKO mice.

Podocyte sEH Deficiency Improves Kidney Function and Blood Pressure Under Hyperglycemic Conditions.

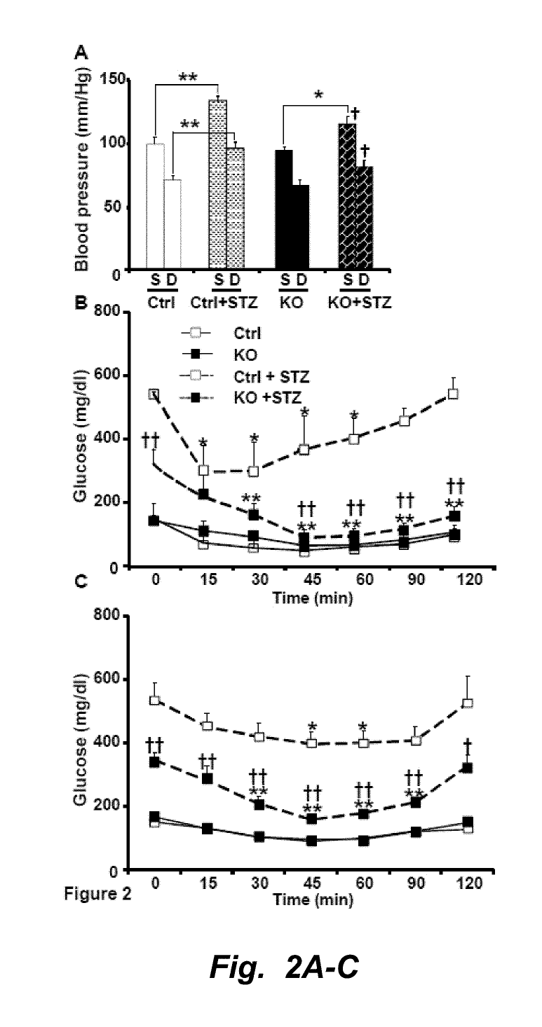

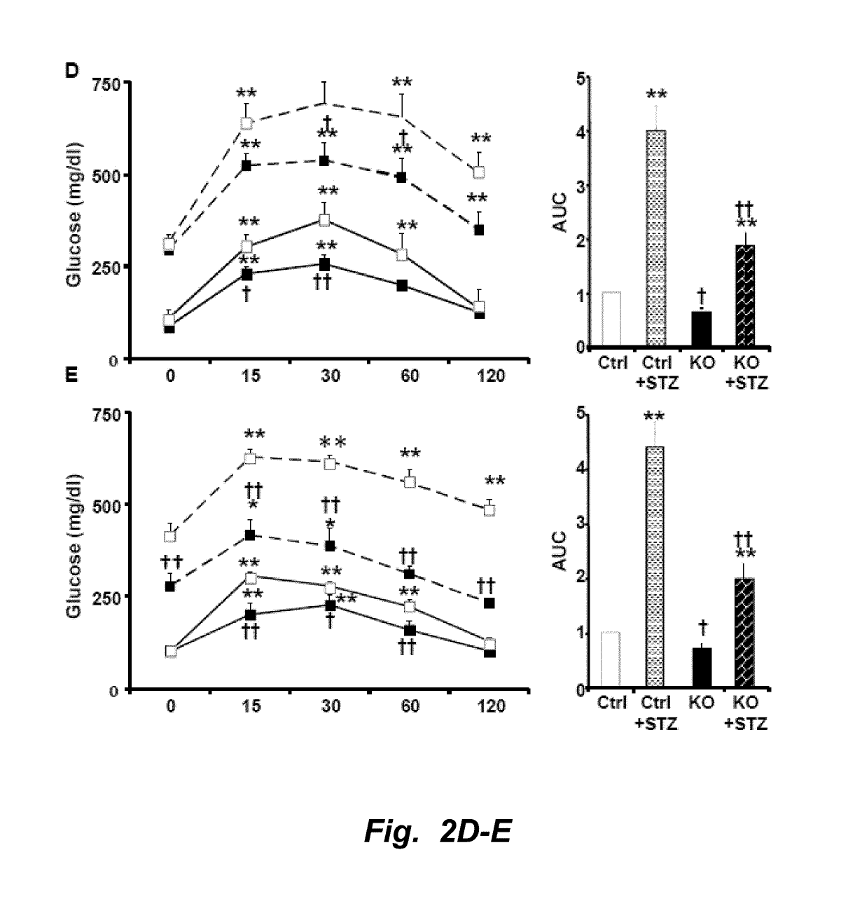

Diabetic nephropathy is characterized by proteinuria and progressive renal failure [28]. In addition, podocytes are involved in the early onset of type 1 and type 2 diabetes [29, 30]. These observations indicate that DN is an excellent model where the molecular events contributing to podocyte damage can be studied in animals. The effects of podocyte sEH deletion on kidney function were evaluated in the established STZ model of DN. Control and pod-sEHKO mice exhibited similar body weights while STZ treatment led to comparable decrease in weight of control and pod-sEHKO mice (Table 4). Kidney weights were increased in STZ-treated animals but to a lesser extent in pod-sEHKO mice. A key monitor for renal injury is albuminuria which is an early and sensitive marker of kidney damage in many types of chronic kidney diseases [31, 32]. In addition, creatinine concentration is a marker for impaired kidney function and for estimated glomerular filtration rate. Serum albumin and creatinine levels were comparable between controls and knockout mice before induction of diabetes. Notably, pod-sEHKO mice exhibited significantly less STZ-induced decrease in serum albumin and increase in serum creatinine compared with controls (Table 4). Consistent with these findings, urine albumin/urine creatinine was lower in pod-sEHKO mice compared with controls. In addition, blood pressure was measured using the tail cuff approach as detailed in Methods [33]. There was no significant difference in basal blood pressure between control and pod-sEHKO mice consistent with findings from whole-body Ephx2 KO mice (FIG. 2A) [21]. Induction of diabetes produced mild but significant increase in blood pressure of control mice. Importantly, hyperglycemia-induced increase in blood pressure was significantly less in pod-sEHKO mice compared with controls (FIG. 2A). Comparable findings were observed in two other independent cohorts of mice. These studies demonstrate protective effects of sEH podocyte deficiency on kidney function and blood pressure under hyperglycemic conditions, and establish podocytes as key and significant contributor to the renal protective effects of sEH deficiency.

Improved Glucose Homeostasis in Podocyte sEH-Deficient Mice.

Kidneys play an important role in regulating glucose homeostasis [34]. In addition, sEH deficiency and pharmacological inhibition in mice improve glucose tolerance [35, 36]. We determined the effects of podocyte sEH deficiency on glucose homeostasis under normal and hyperglycemic conditions. Fasted serum glucose concentration was significantly lower in pod-sEHKO mice compared with controls under hyperglycemic condition (Table 4).

TABLE 4
Metabolic parameters of control and pod-sEHKO mice

| | Ctrl | Ctrl + STZ | KO | KO + STZ |
|---|---|---|---|---|
| | (n = 6) | (n = 7) | (n = 6) | (n = 8) |
| Body weight (g) | 28.09 ± 0.17 | 25.32 ± 0.28** | 28.76 ± 0.26 | 25.89 ± 0.19** |
| Kidney weight (g) | 0.35 ± 0.01 | 0.43 ± 0.01** | 0.33 ± 0.01 | 0.38 ± 0.01**^{††} |
| Serum albumin (mg/dl) | 20.3 ± 2.4 | 11.5 ± 1.3** | 22.6 ± 1.6 | 15.7 ± 2.3**^{††} |
| Serum creatinine (mg/dl) | 0.30 ± 0.05 | 0.47 ± 0.03** | 0.28 ± 0.03 | 0.37 ± 0.02**^{††} |
| Urine Albumin/Creatinine (μg/ml) | ND | 47.33 ± 4.3** | ND | 36.0 ± 2.7**^{††} |
| Fasted serum glucose (mg/dl) | 89.8 ± 5.7 | 313.3 ± 19.5** | 80.5 ± 6.2 | 247.6 ± 16.6**^{†} |
| Fed serum glucose (mg/dl) | 164.3 ± 9.6 | 416.9 ± 33.3** | 146.1 ± 7.1^{†} | 361.1 ± 22.2**^{††} |
| Fasted urine glucose (mg/dl) | ND | 299.1 ± 23.5** | ND | 377.6 ± 33.3**^{††} |
| Fed urine glucose (mg/dl) | ND | 511.8 ± 44.7** | ND | 658.17 ± 17.3**^{††} |
| HDL-C (mg/dl) | 92.7 ± 1.2 | 74.6 ± 2.3** | 64.2 ± 0.8^{††} | 131.7 ± 2.1**^{††} |

Plasma and urine levels of albumin, creatinine and glucose at fed or fasted states in control and pod-sEHKO mice without and with STZ at 24 weeks after injection.
*p < 0.05;
**p < 0.01 without vs with STZ, and
^{†}p < 0.05;
^{††}p < 0.01 Ctrl vs KO.

Similarly, fed serum glucose was significantly lower in pod-sEHKO mice than controls under basal and hyperglycemic conditions. In addition, no glucose was detected in urine under basal conditions as expected. Importantly, under hyperglycemic conditions fasted and fed urine glucose concentrations were significantly higher in pod-sEHKO mice compared with controls indicative of enhanced clearance (Table 4). Moreover, fed high density lipoprotein cholesterol (HDL) concentration was higher in pod-sEHKO mice compared with controls under hyperglycemic conditions. To directly assess insulin sensitivity, mice were subjected to ITTs at 2 and 15 weeks after STZ injection. Basally (without STZ) control and pod-sEHKO mice exhibited comparable insulin sensitivity (FIG. 2B, C). However, upon STZ treatment pod-sEHKO mice exhibited improved insulin sensitivity compared with controls. To determine glucose tolerance mice were subjected to GTTs at 3 and 16 weeks after STZ injection. Under basal condition pod-sEHKO mice displayed moderate increase in ability to clear glucose from peripheral circulation compared with controls suggesting enhanced glucose tolerance. Further, pod-sEHKO mice displayed significantly enhanced glucose tolerance compared with controls under hyperglycemic condition (FIG. 2D, E). Renal gluconeogenesis is a significant contributor to glucose homeostasis under normal and hyperglycemic conditions [37, 38]. Semi-quantitative RTPCR was used to determine the effects of podocyte sEH deficiency on expression of genes implicated in gluconeogenesis in liver and kidney. Hyperglycemia induced significant increase in fed phosphoenolpyruvate carboxykinase (PEPCK) and glucose 6 phosphatase (G6Pase) mRNA in control mice (FIG. 2F, G). Consistent with improved glucose tolerance pod-sEHKO mice exhibited significantly less hyperglycemia-induced expression of PEPCK and G6Pase in liver and kidney. Together, these findings demonstrate that podocyte sEH deficiency leads to mild and pronounced improvement in glucose homeostasis under basal and hyperglycemic conditions, respectively.

Podocyte sEH Deficiency Attenuates Hyperglycemia-Induced Glomeruloscelerosis.

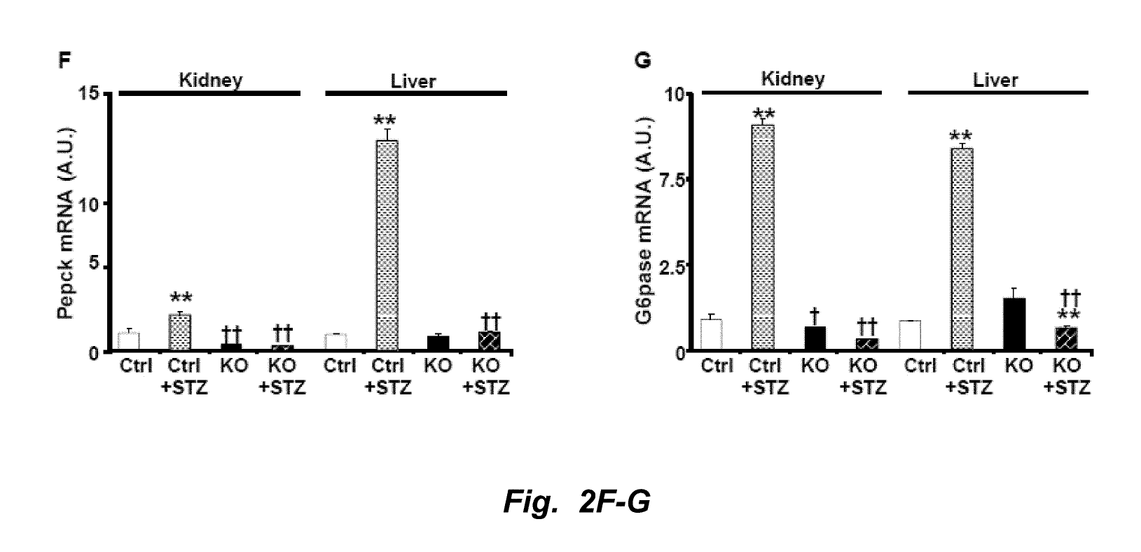

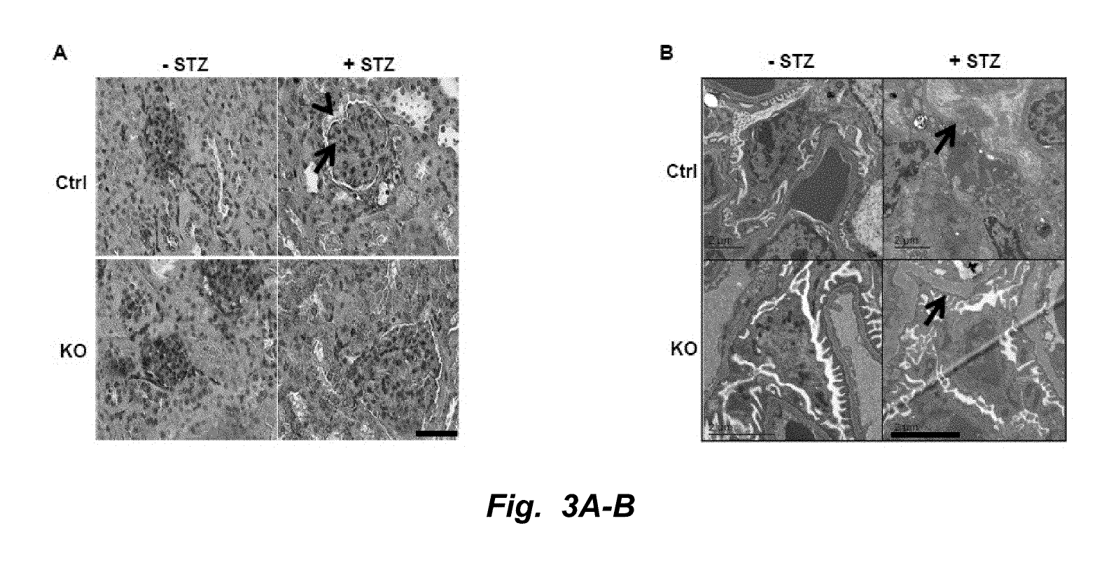

Ephx2 deficiency and sEH pharmacological inhibition prevent renal interstitial fibrosis in unilateral ureteral obstruction model [19, 20]. The effects of sEH podocyte deficiency on glomerulosclerosis were determined using Periodic acid-Schiff (PAS) staining (FIG. 3A). While no significant differences were noted under basal conditions, hyperglycemia caused severe damage to the kidneys of control mice but to a lesser extent in pod-sEHKO as evidenced by distorted architecture of the glomerules and tubules, flattened epithelia and nuclear and epithelial debris in the lumina. In addition, Kimmelstiel-Wilson lesion nodules (black arrow) were observed at higher frequency in STZ-treated controls compared with knockouts (FIG. 3A). Moreover, electron microscopy revealed significant alterations in the morphology of podocytes upon STZ administration (FIG. 3B). Swollen podocytes with large cytoplasmic vacuoles and effaced foot processes (arrow) were observed in STZ treated control mice. In contrast, pod-sEHKO mice exhibited mild focal foot process effacement indicative of lower STZ-induced renal damage. These data suggest that sEH deficiency protects podocyte structure and foot processes against hyperglycemia-induced toxicity.

Podocyte sEH Deficiency Mitigates Hyperglycemia-Induced Endoplasmic Reticulum Stress and Inflammation.

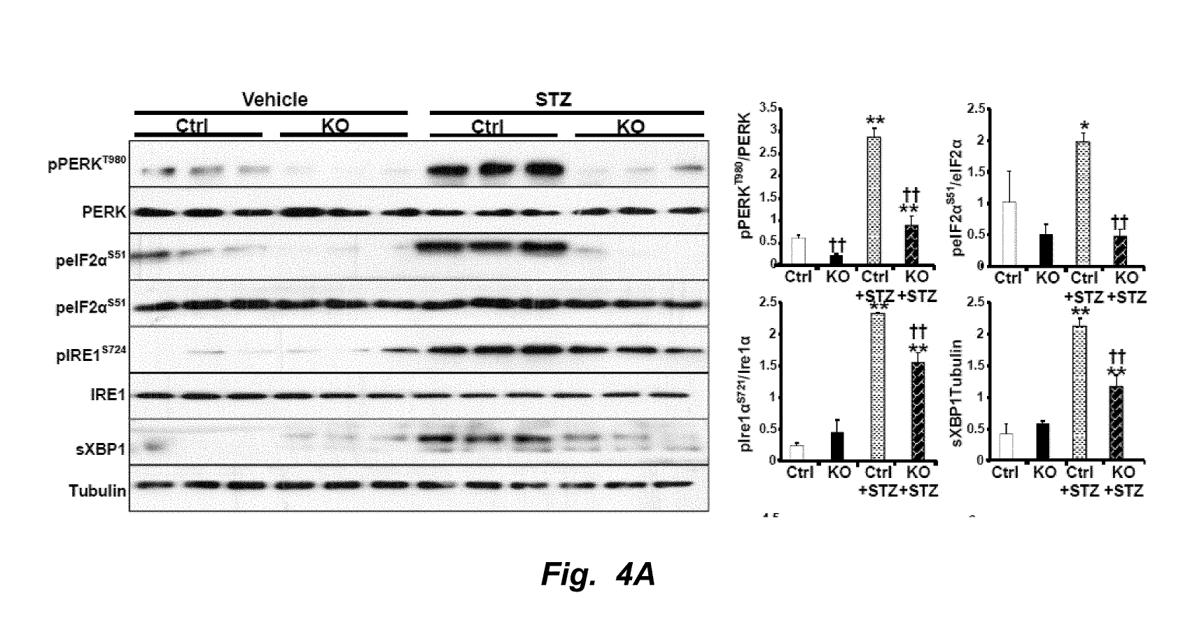

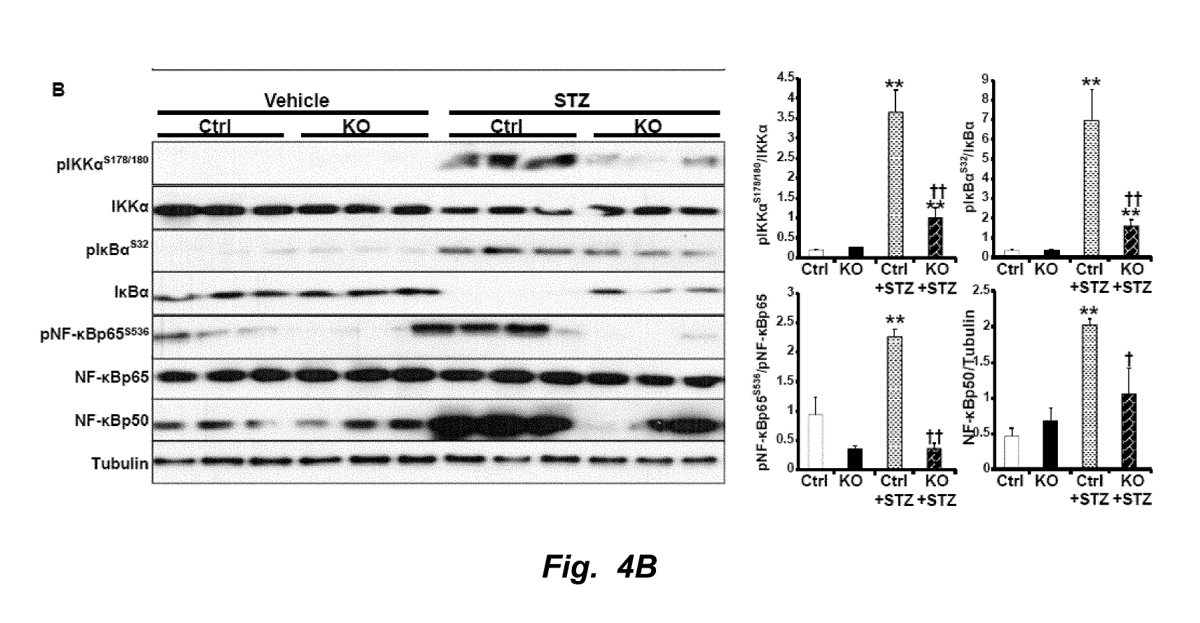

The ER plays an important role in folding of newly synthesized proteins and in humans it is estimated that protein synthesis by the kidneys is ˜40% of total daily load [39] indicating that kidney cells could be highly susceptible to ER stress. ER stress is implicated in the pathogenesis of kidney disease and DN [40], and sEH deficiency and inhibition attenuate ER stress [41-44]. The effects of sEH podocyte deficiency on ER stress were determined in control and pod-sEHKO mice under normal and hyperglycemic conditions. We evaluated activation of ER transmembrane proteins PKR-like ER-regulated kinase (PERK) and inositol requiring enzyme 1α (IRE1α), and their downstream targets α-subunit of eukaryotic translation initiation factor 2 (eIF2α) and X-box binding protein 1 (XBP1), respectively [45, 46]. Hyperglycemia induced ER stress as evidenced by increased PERK (Thr⁹⁸⁰), eIF2α (Ser⁵¹) and IRE1α (Ser⁷²⁴) phosphorylation, and sXBP1 expression (FIG. 4A). Under basal conditions pod-sEHKO mice exhibited mild attenuation of ER stress compared with controls. Importantly, pod-sEHKO mice exhibited significant attenuation of ER stress compared with controls under hyperglycemic conditions (FIG. 4A). sEH deficiency and pharmacological inhibition exhibit anti-inflammatory effects through NF-κB inhibition [47]. Accordingly, we determined the activation of NF-κB signaling in control and pod-sEHKO mice. Notably, hyperglycemia-induced IKKα, IₖBα and NF-κBp65 phosphorylation and NF-κBp50 expression were decreased in pod-sEHKO mice compared with controls (FIG. 4B). Collectively, these data establish that podocyte sEH deficiency attenuates hyperglycemia-induced ER stress and inflammation.

Podocyte sEH Deficiency Enhances Autophagy and Attenuates Hyperglycemia-Induced Fibrosis.

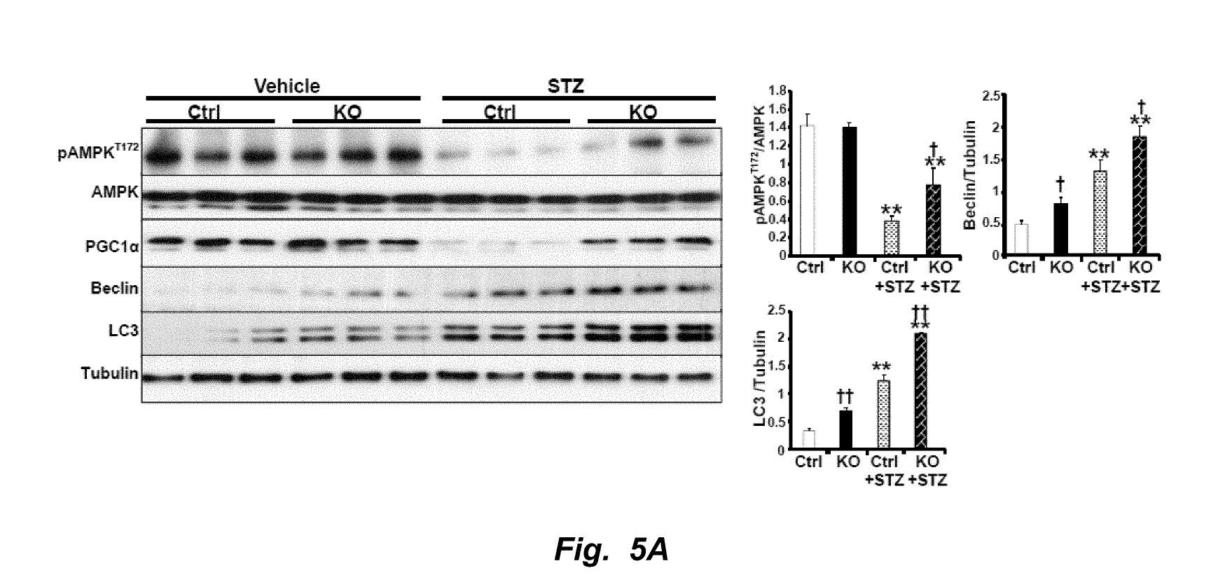

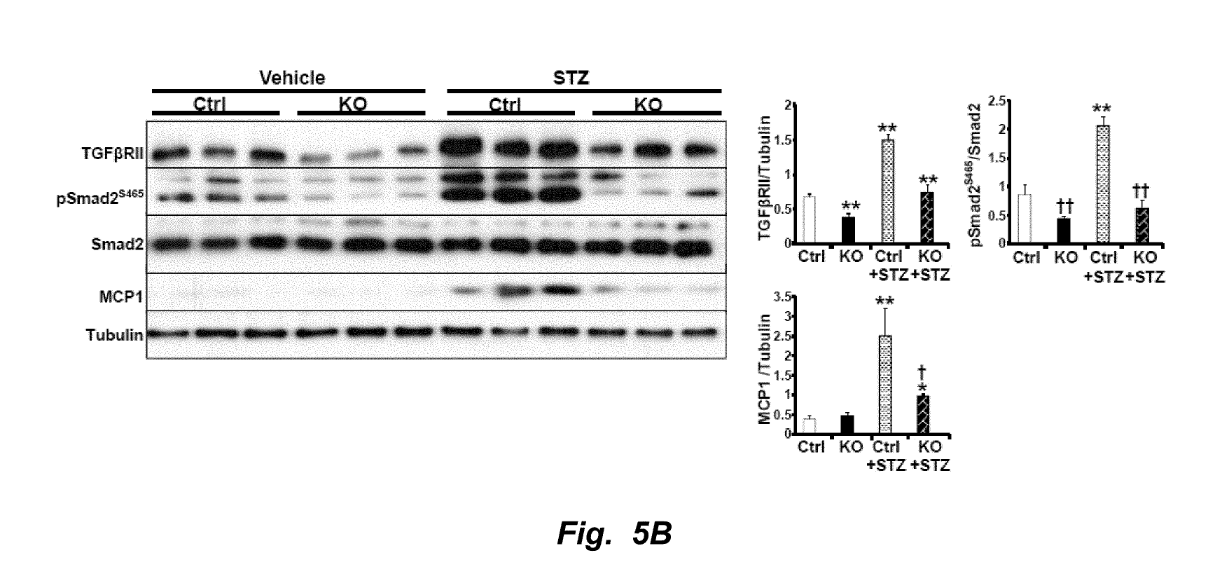

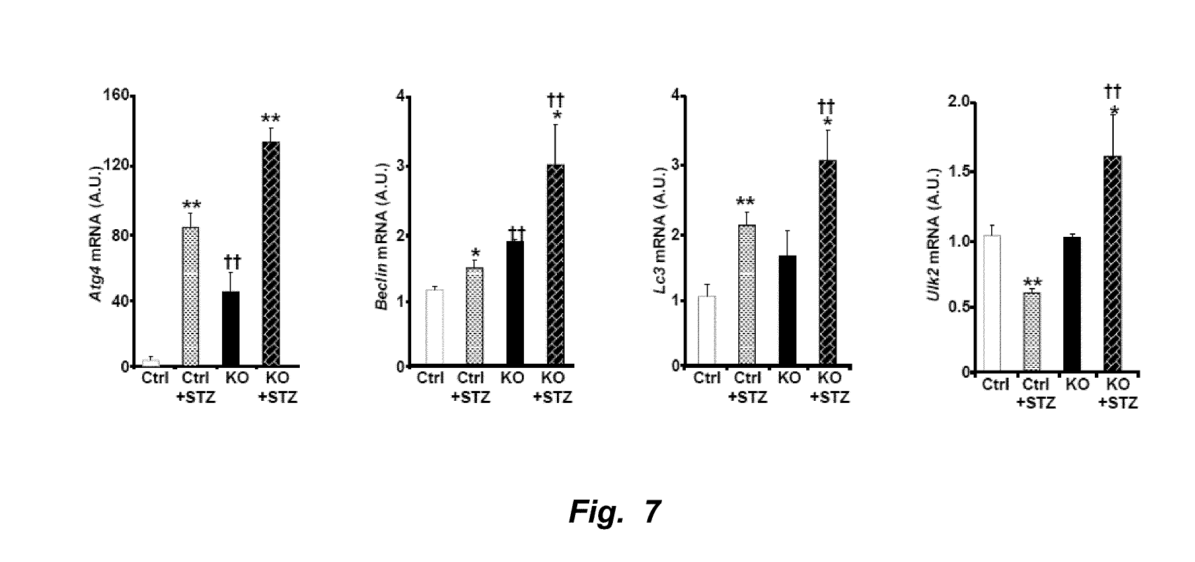

Autophagy is a multi-step, well-coordinated fundamental cell process that delivers intracellular constituents to lysosomes for degradation to maintain homeostasis [48]. Accumulating evidence implicates autophagy in regulating critical aspects of normal and diabetic kidney [49, 50]. In the diabetic kidney autophagy is regulated by several molecular modulators including AMP-activated protein kinase (AMPK) and mTOR complex 1 (mTORC1). AMPK is a nutrient sensing kinase and is a potent positive regulator of autophagy [51-53], while mTORC1 is a negative regulator of autophagy [54, 55]. The effects of sEH podocyte deficiency on autophagy were evaluated in control and pod-sEHKO mice under normal and hyperglycemic conditions. In line with published reports [51, 52, 56] hyperglycemia decreased AMPK activation and phosphorylation (Thr¹⁷²) but to a lesser level in pod-sEHKO compared with controls (FIG. 5A). Similarly, pod-sEHKO mice exhibited less hyperglycemia-induced downregulation of PGC1α expression. PGC1α is required for AMPK action on gene expression in several tissues including kidney [57]. Additionally, pod-sEHKO mice exhibited enhanced autophagy compared with controls under basal and hyperglycemia conditions as evidenced by increased Beclin1 and microtubule-associated protein 1A/1B-light chain 3 (LC3) expression [58, 59] (FIG. 5A). In line with these findings mRNA of beclin, Lc3 and additional markers of autophagy cysteine protease ATG4D (Atg4) [60] and Unc-51-like kinase 2 (Ulk2) [61] were similarly enhanced in pod-sEHKO mice under hyperglycemic conditions (FIG. 7). Consistent with enhanced autophagy, pod-sEHKO mice exhibited decreased hyperglycemic-induced fibrosis compared with controls as evidenced by decreased TGFβRII expression and decreased phosphorylation of Smad2 [62, 63]. Collectively, these findings demonstrate that podocyte sEH deficiency leads to enhanced autophagy with corresponding decrease in fibrosis.

Decreased ER Stress and Enhanced Autophagy in Differentiated E11 Podocytes with Pharmacological Inhibition of sEH.

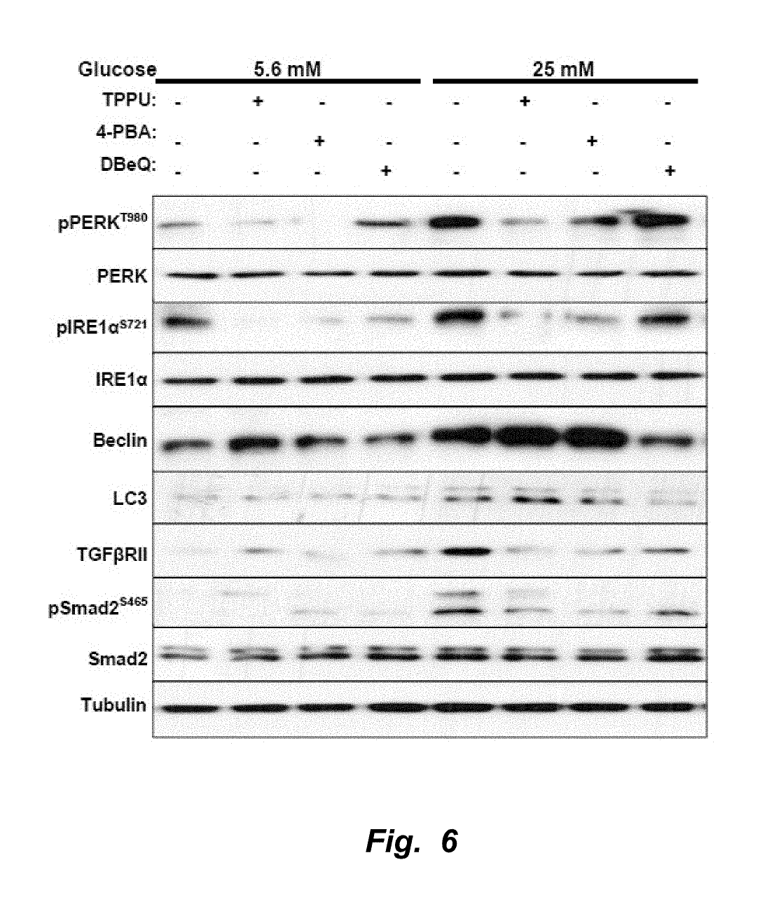

To determine if effects of podocyte-sEH deletion in vivo were cell autonomous differentiated podocytes were treated with selective sEH pharmacological inhibitor. Differentiated culture mouse E11 podocytes were treated with 1-trifluoromethoxyphenyl-3-(1-propionylpiperidin-4-yl) urea (TPPU) as detailed in Methods. TPPU is an effective inhibitor of sEH with an IC50 of 1.1 and 2.1 nM for murine and human sEH, respectively [64-66]. Recently we demonstrated that sEH inhibition using TPPU recapitulates the effects of sEH deficiency on acute pancreatitis in mice [43, 44]. Alterations in ER stress, autophagy and fibrosis were biochemically evaluated in differentiated podocytes treated with TPPU, ER stress inhibitor 4-phenybutyrate (4-PBA) [67] and autophagy inhibitor N2,N4-dibenzylquinazoline-2,4-diamine (DBeQ) [68] under normal (5.6 mM) and high (25 mM) glucose conditions. TPPU treated podocytes exhibited decreased ER stress, enhanced autophagy and attenuated fibrosis compared with controls under basal and high glucose conditions (FIG. 6). These findings are in line with observations in pod-sEHKO kidney lysates and suggest that the effects of podocyte sEH deletion are likely cell-autonomous.

### DISCUSSION

Diabetic nephropathy is the leading cause of end stage kidney disease and podocyte dysfunction plays a significant role in the pathogenesis of DN. Elucidating the mechanisms underlying podocyte function is critical for understanding disease pathogenesis and developing better therapies. In the current study, we investigated the role of sEH in podocyte function under normoglycemic and hyperglycemic conditions. Podocyte sEH deficient mice exhibited moderate improvement in kidney function and systemic glucose homeostasis in a normoglycemic environment, and the salutary effects of podocyte sEH deficiency were significantly improved under hyperglycemic condition. This was associated with cell-autonomous decrease in endoplasmic reticulum stress and enhanced autophagy with corresponding decrease in inflammation and fibrosis in the kidney. Collectively, these findings identify sEH in podocytes as a significant contributor to kidney function and may have potential therapeutic implications.

Using a genetic approach, we demonstrated that sEH deficiency in podocytes ameliorated the effects of hyperglycemia as evidenced by improved kidney function and blood pressure and systemic glucose homeostasis. We observed increased sEH expression in podocytes under hyperglycemic conditions, and enhanced sEH expression is often associated with inflammation [11]. It is not clear if changes in sEH expression in podocytes are correlative or underlie disease pathogenesis but suggest that dysregulation of sEH signaling in podocytes may be relevant to renal function and DN. We utilized podocin-Cre mice to selectively disrupt sEH expression in podocytes since this Cre transgenic line achieves efficient and selective deletion as previously reported [23]. Indeed, biochemical studies on tissues and isolated podocytes as well as immunohistochemistry presented herein are consistent with efficient and selective deletion of sEH in podocytes using this strategy. Remarkably, podocyte sEH deficiency improved kidney function and significantly reduced renal injury during diabetes. Decreased hyperglycemia-induced albuminuria and blood pressure in pod-sEHKO mice are in line with the renal protective effects of whole-body Ephx2 deletion [22], and establish podocytes as major contributors to the renal protective effects of sEH deficiency. Diabetic albuminuria in humans is associated with the development of characteristic histopathologic features, including glomerular hypertrophy and thickening of the glomerular basement membrane [69]. Consistent with the decreased albuminuria in pod-sEHKO mice PAS staining and electron microscopy reveal mild focal foot process effacement indicative of lower hyperglycemia-induced renal damage. These findings suggest that podocyte sEH inhibition may be a valuable approach to prevent glomerular and podocyte injury. Moreover, we demonstrate for the first time improved systemic glucose homeostasis in podocyte-sEH deficient mice. The kidneys play an important role in regulating glucose homeostasis through gluconeogenesis, glucose utilization and glucose reabsorption via glucose transporters and sodium glucose cotransporters (SGTLs) [34]. Indeed, selective inhibition of SGTL2 emerged as novel therapy that lowers plasma glucose levels by reducing reabsorption of filtered glucose in patients with T2D [70-72]. Improved glucose homeostasis in pod-sEHKO mice is likely due to: 1) Increased glucosuria in pod-sEHKO mice indicative of enhanced glucose clearance under diabetic conditions, and 2) Improved renal gluconeogenesis as evidenced by attenuated PEPCK and G6pase expression in pod-sEHKO. It is worth noting that in healthy individuals the kidneys contribute 20%-25% of the glucose released into circulation via gluconeogenesis [37]. However, in patients with T2D both hepatic and renal gluconeogenesis are increased, but the relative increase in renal gluconeogenesis is substantially greater than hepatic gluconeogenesis (300% vs. 30%) [38]. We cannot rule out that sEH deletion in podocytes affects other tissue(s) (such as liver) that contribute significantly to glucose homeostasis. These novel findings have potentially significant translational implications and raise the possibility of deploying sEH inhibitors to treat patients with T2D (inadequately controlled with other glucose-lowering drugs) as monotherapy and/or in combination with other drugs such as SGTL2 inhibitors.

At the molecular level, podocyte sEH deficiency led to cell-autonomous attenuation of ER stress and enhanced autophagy with corresponding decrease in NF-□B inflammatory response and fibrosis. The kidney cells are highly susceptible to stress and ER stress has been implicated in the pathogenesis of kidney disease and DN [40]. Importantly, chemical chaperones that inhibit ER stress 4-PBA [73] and TUDCA [74] slow DN disease progression. Attenuated basal and hyperglycemia-induced ER stress in pod-sEHKO mice is consistent with previous findings demonstrating that sEH deficiency and pharmacological inhibition attenuate HFD-induced ER stress in liver and adipose tissue [41, 42], and in pancreas during acute pancreatitis [43, 44]. In line with decreased ER stress, podocyte sEH deficiency attenuated hypeglycemia-induced NF-κB inflammatory response. Hence it is reasonable to stipulate that the renal protective effects of podocyte sEH deficiency are mediated, at least partly, by attenuated ER stress. A growing body of evidence underscores the importance of autophagy as a protective mechanism against podocyte injury. Autophagy regulates many critical aspects of normal and diabetic kidney [49, 50]. Cellular autophagy is inhibited in kidneys of STZ-induced diabetic rodents [75-77], and impaired autophagy is observed in kidney samples of T2D patients [78]. In addition, podocytes exhibit a high level of autophagy which may serve as a mechanism for maintaining their cellular homeostasis [79, 80]. Conceivably, sEH can regulate autophagy directly or indirectly through modulating effector(s) such as AMPK. Regardless of the prcise mechanism enhanced autophagy in pod-sEH KO mice is consistent with decreased fibrosis and is likely a significant contributor to the renal protective effects of podocyte deficiency.

The current studies uncover a novel role for sEH in podocytes and identify podocytes as major and significant contributor to the renal protective effects of sEH deficiency. These findings suggest that sEH inhibition in podocytes may represent a potential approach for improving kidney function and treating diabetic nephropathy.

### REFERENCES

1. Shi, Y. and F. B. Hu, The global implications of diabetes and cancer. Lancet, 2014. 383(9933): p. 1947-8.
2. de Boer, I. H., et al., Central obesity, incident microalbuminuria, and change in creatinine clearance in the epidemiology of diabetes interventions and complications study. J Am Soc Nephrol, 2007. 18(1): p. 235-43.
3. Rivero, A., et al., Pathogenic perspectives for the role of inflammation in diabetic nephropathy. Clin Sci (Lond), 2009. 116(6): p. 479-92.
4. Sassy-Prigent, C., et al., Early glomerular macrophage recruitment in streptozotocin-induced diabetic rats. Diabetes, 2000. 49(3): p. 466-75.
5. Pavenstadt, H., W. Kriz, and M. Kretzler, Cell biology of the glomerular podocyte. Physiol Rev, 2003. 83(1): p. 253-307.
6. Lavin, P. J., et al., Therapeutic targets in focal and segmental glomerulosclerosis. Curr Opin Nephrol Hypertens, 2008. 17(4): p. 386-92.
7. Brown, E. J., et al., Mutations in the formin gene INF2 cause focal segmental glomerulosclerosis. Nat Genet, 2010. 42(1): p. 72-6.
8. Wolf, G., S. Chen, and F. N. Ziyadeh, From the periphery of the glomerular capillary wall toward the center of disease: podocyte injury comes of age in diabetic nephropathy. Diabetes, 2005. 54(6): p. 1626-34.
9. Spector, A. A. and A. W. Norris, Action of epoxyeicosatrienoic acids on cellular function. Am J Physiol Cell Physiol, 2007. 292(3): p. C996-1012.
10. Node, K., et al., Anti-inflammatory properties of cytochrome P450 epoxygenase-derived eicosanoids. Science, 1999. 285(5431): p. 1276-9.
11. Imig, J. D., et al., Soluble epoxide hydrolase inhibition lowers arterial blood pressure in angiotensin II hypertension. Hypertension, 2002. 39(2 Pt 2): p. 690-4.
12. Wagner, K., et al., Epoxygenated fatty acids and soluble epoxide hydrolase inhibition: novel mediators of pain reduction. J Agric Food Chem. 59(7): p. 2816-24.
13. Yu, Z., et al., Soluble epoxide hydrolase regulates hydrolysis of vasoactive epoxyeicosatrienoic acids. Circ Res, 2000. 87(11): p. 992-8.
14. Enayetallah, A. E., et al., Distribution of soluble epoxide hydrolase and of cytochrome P450 2C8, 2C9, and 2J2 in human tissues. J Histochem Cytochem, 2004. 52(4): p. 447-54.
15. Newman, J. W., C. Morisseau, and B. D. Hammock, Epoxide hydrolases: their roles and interactions with lipid metabolism. Prog Lipid Res, 2005. 44(1): p. 1-51.
16. Imig, J. D., et al., An orally active epoxide hydrolase inhibitor lowers blood pressure and provides renal protection in salt-sensitive hypertension. Hypertension, 2005. 46(4): p. 975-81.
17. Olearczyk, J. J., et al., Administration of a substituted adamantyl urea inhibitor of soluble epoxide hydrolase protects the kidney from damage in hypertensive Goto-Kakizaki rats. Clin Sci (Lond), 2009. 116(1): p. 61-70.
18. Zhao, X., et al., Soluble epoxide hydrolase inhibition protects the kidney from hypertension-induced damage. J Am Soc Nephrol, 2004. 15(5): p. 1244-53.
19. Kim, J., et al., Inhibition of soluble epoxide hydrolase prevents renal interstitial fibrosis and inflammation. Am J Physiol Renal Physiol, 2014. 307(8): p. F971-80.
20. Kim, J., et al., Pharmacological inhibition of soluble epoxide hydrolase prevents renal interstitial fibrogenesis in obstructive nephropathy. Am J Physiol Renal Physiol, 2015. 308(2): p. F131-9.
21. Manhiani, M., et al., Soluble epoxide hydrolase gene deletion attenuates renal injury and inflammation with DOCA-salt hypertension. Am J Physiol Renal Physiol, 2009. 297(3): p. F740-8.
22. Elmarakby, A. A., et al., Deletion of soluble epoxide hydrolase gene improves renal endothelial function and reduces renal inflammation and injury in streptozotocin-induced type 1 diabetes. Am J Physiol Regul Integr Comp Physiol, 2011. 301(5): p. R1307-17.
23. Welsh, G. I., et al., Insulin signaling to the glomerular podocyte is critical for normal kidney function. Cell Metab, 2010. 12(4): p. 329-40.
24. Gomathi, D., et al., Evaluation of antioxidants in the kidney of streptozotocin induced diabetic rats. Indian J Clin Biochem, 2014. 29(2): p. 221-6.
25. Saito, T., et al., Nucleobindin-2 is a positive regulator for insulin-stimulated glucose transporter 4 translocation in fenofibrate treated E11 podocytes. Endocr J, 2014. 61(9): p. 933-9.
26. Oguro, A., N. Fujita, and S. Imaoka, Regulation of soluble epoxide hydrolase (sEH) in mice with diabetes: high glucose suppresses sEH expression. Drug Metab Pharmacokinet, 2009. 24(5): p. 438-45.
27. Chen, G., et al., Genetic disruption of soluble epoxide hydrolase is protective against streptozotocin-induced diabetic nephropathy. Am J Physiol Endocrinol Metab, 2012. 303(5): p. E563-75.
28. Wolf, G. and F. N. Ziyadeh, Cellular and molecular mechanisms of proteinuria in diabetic nephropathy. Nephron Physiol, 2007. 106(2): p. p 26-31.
29. Pagtalunan, M. E., et al., Podocyte loss and progressive glomerular injury in type II diabetes. J Clin Invest, 1997. 99(2): p. 342-8.
30. Coward, R. and A. Fornoni, Insulin signaling: implications for podocyte biology in diabetic kidney disease. Curr Opin Nephrol Hypertens, 2015. 24(1): p. 104-10.
31. Lopez-Giacoman, S. and M. Madero, Biomarkers in chronic kidney disease, from kidney function to kidney damage. World J Nephrol, 2015. 4(1): p. 57-73.
32. Wasung, M. E., L. S. Chawla, and M. Madero, Biomarkers of renal function, which and when? Clin Chim Acta, 2015. 438: p. 350-7.
33. Kubota, Y., et al., Evaluation of blood pressure measured by tail-cuff methods (without heating) in spontaneously hypertensive rats. Biol Pharm Bull, 2006. 29(8): p. 1756-8.
34. Wilding, J. P., The role of the kidneys in glucose homeostasis in type 2 diabetes: clinical implications and therapeutic significance through sodium glucose co-transporter 2 inhibitors. Metabolism, 2014. 63(10): p. 1228-37.
35. Luria, A., et al., Soluble epoxide hydrolase deficiency alters pancreatic islet size and improves glucose homeostasis in a model of insulin resistance. Proc Natl Acad Sci USA, 2011. 108(22): p. 9038-43.
36. Bettaieb, A., et al., Soluble epoxide hydrolase deficiency or inhibition attenuates diet-induced endoplasmic reticulum stress in liver and adipose tissue. J Biol Chem, 2013. 288(20): p. 14189-99.
37. Stumvoll, M., et al., Uptake and release of glucose by the human kidney. Postabsorptive rates and responses to epinephrine. J Clin Invest, 1995. 96(5): p. 2528-33.
38. Gerich, J. E., Role of the kidney in normal glucose homeostasis and in the hyperglycaemia of diabetes mellitus: therapeutic implications. Diabet Med, 2010. 27(2): p. 136-42.
39. Tessari, P., et al., Kidney, splanchnic, and leg protein turnover in humans. Insight from leucine and phenylalanine kinetics. J Clin Invest, 1996. 98(6): p. 1481-92.
40. Zhuang, A. and J. M. Forbes, Stress in the kidney is the road to pERdition: is endoplasmic reticulum stress a pathogenic mediator of diabetic nephropathy? J Endocrinol, 2014. 222(3): p. R97-111.
41. Bettaieb, A., et al., Soluble Epoxide Hydrolase Deficiency or Inhibition Attenuates Diet-Induced Endoplasmic Reticulum Stress in Liver and Adipose Tissue. J Biol Chem.
42. Harris, T. R., et al., Inhibition of soluble epoxide hydrolase attenuates hepatic fibrosis and endoplasmic reticulum stress induced by carbon tetrachloride in mice. Toxicol Appl Pharmacol, 2015.
43. Bettaieb, A., et al., Effects of soluble epoxide hydrolase deficiency on acute pancreatitis in mice. PLoS One, 2014. 9(11): p. e113019.
44. Bettaieb, A., et al., Soluble Epoxide Hydrolase Pharmacological Inhibition Ameliorates Experimental Acute Pancreatitis in Mice. Mol Pharmacol, 2015.
45. Ron, D. and P. Walter, Signal integration in the endoplasmic reticulum unfolded protein response. Nat Rev Mol Cell Biol, 2007. 8(7): p. 519-29.
46. Hotamisligil, G. S., Endoplasmic reticulum stress and the inflammatory basis of metabolic disease. Cell. 140(6): p. 900-17.
47. Shen, H. C. and B. D. Hammock, Discovery of inhibitors of soluble epoxide hydrolase: a target with multiple potential therapeutic indications. J Med Chem. 55(5): p. 1789-808.
48. Ravikumar, B., et al., Regulation of mammalian autophagy in physiology and pathophysiology. Physiol Rev, 2010. 90(4): p. 1383-435.
49. Wang, Z. and M. E. Choi, Autophagy in kidney health and disease. Antioxid Redox Signal, 2014. 20(3): p. 519-37.
50. Ding, Y. and M. E. Choi, Autophagy in diabetic nephropathy. J Endocrinol, 2015. 224(1): p. R15-30.
51. Lee, M. J., et al., A role for AMP-activated protein kinase in diabetes-induced renal hypertrophy. Am J Physiol Renal Physiol, 2007. 292(2): p. F617-27.
52. Kitada, M., et al., Resveratrol improves oxidative stress and protects against diabetic nephropathy through normalization of Mn-SOD dysfunction in AMPK/SIRT1-independent pathway. Diabetes, 2011. 60(2): p. 634-43.
53. Sokolovska, J., et al., Influence of metformin on GLUT1 gene and protein expression in rat streptozotocin diabetes mellitus model. Arch Physiol Biochem, 2010. 116(3): p. 137-45.
54. Lloberas, N., et al., Mammalian target of rapamycin pathway blockade slows progression of diabetic kidney disease in rats. J Am Soc Nephrol, 2006. 17(5): p. 1395-404.
55. Sakaguchi, M., et al., Inhibition of mTOR signaling with rapamycin attenuates renal hypertrophy in the early diabetic mice. Biochem Biophys Res Commun, 2006. 340(1): p. 296-301.
56. Ding, D. F., et al., Resveratrol attenuates renal hypertrophy in early-stage diabetes by activating AMPK. Am J Nephrol, 2010. 31(4): p. 363-74.
57. Weinberg, J. M., Mitochondrial biogenesis in kidney disease. J Am Soc Nephrol, 2011. 22(3): p. 431-6.
58. Ma, T., et al., High glucose induces autophagy in podocytes. Exp Cell Res, 2013. 319(6): p. 779-89.
59. Xiao, T., et al., Rapamycin promotes podocyte autophagy and ameliorates renal injury in diabetic mice. Mol Cell Biochem, 2014. 394(1-2): p. 145-54.
60. Cybulsky, A. V., The intersecting roles of endoplasmic reticulum stress, ubiquitin-proteasome system, and autophagy in the pathogenesis of proteinuric kidney disease. Kidney Int, 2013. 84(1): p. 25-33.
61. Takabatake, Y., et al., Autophagy and the kidney: health and disease. Nephrol Dial Transplant, 2014. 29(9): p. 1639-47.
62. Huang, Y. R., et al., Ureic clearance granule, alleviates renal dysfunction and tubulointerstitial fibrosis by promoting extracellular matrix degradation in renal failure rats, compared with enalapril. J Ethnopharmacol, 2014. 155(3): p. 1541-52.
63. Fukasawa, H., et al., Treatment with anti-TGF-beta antibody ameliorates chronic progressive nephritis by inhibiting Smad/TGF-beta signaling. Kidney Int, 2004. 65(1): p. 63-74.
64. Rose, T. E., et al., 1-Aryl-3-(1-acylpiperidin-4-yl)urea inhibitors of human and murine soluble epoxide hydrolase: structure-activity relationships, pharmacokinetics, and reduction of inflammatory pain. J Med Chem. 53(19): p. 7067-75.
65. Tsai, H. J., et al., Pharmacokinetic screening of soluble epoxide hydrolase inhibitors in dogs. Eur J Pharm Sci. 40(3): p. 222-38.
66. Liu, J. Y., et al., Substituted phenyl groups improve the pharmacokinetic profile and anti-inflammatory effect of urea-based soluble epoxide hydrolase inhibitors in murine models. Eur J Pharm Sci. 48(4-5): p. 619-27.
67. Carlisle, R. E., et al., 4-Phenylbutyrate inhibits tunicamycin-induced acute kidney injury via CHOP/GADD153 repression. PLoS One, 2014. 9(1): p. e84663.
68. Chou, T. F., et al., Reversible inhibitor of p97, DBeQ, impairs both ubiquitin-dependent and autophagic protein clearance pathways. Proc Natl Acad Sci USA, 2011. 108(12): p. 4834-9.
69. Cox, J. P., E. O'Brien, and K. O'Malley, The J-shaped curve in elderly hypertensives. J Hypertens Suppl, 1992. 10(2): p. S17-23.
70. Zambrowicz, B., et al., LX4211, a dual SGLT1/SGLT2 inhibitor, improved glycemic control in patients with type 2 diabetes in a randomized, placebo-controlled trial. Clin Pharmacol Ther, 2012. 92(2): p. 158-69.
71. Zambrowicz, B., et al., Effects of LX4211, a dual sodium-dependent glucose cotransporters 1 and 2 inhibitor, on postprandial glucose, insulin, glucagon-like peptide 1, and peptide tyrosine tyrosine in a dose-timing study in healthy subjects. Clin Ther, 2013. 35(8): p. 1162-1173 e8.
72. Kurosaki, E. and H. Ogasawara, Ipragliflozin and other sodium-glucose cotransporter-2 (SGLT2) inhibitors in the treatment of type 2 diabetes: preclinical and clinical data. Pharmacol Ther, 2013. 139(1): p. 51-9.
73. Qi, W., et al., Attenuation of diabetic nephropathy in diabetes rats induced by streptozotocin by regulating the endoplasmic reticulum stress inflammatory response. Metabolism, 2011. 60(5): p. 594-603.
74. Chen, Y., et al., Effect of taurine-conjugated ursodeoxycholic acid on endoplasmic reticulum stress and apoptosis induced by advanced glycation end products in cultured mouse podocytes. Am J Nephrol, 2008. 28(6): p. 1014-22.
75. Barbosa Junior Ade, A., et al., Inhibition of cellular autophagy in proximal tubular cells of the kidney in streptozotocin-diabetic and uninephrectomized rats. Virchows Arch B Cell Pathol Incl Mol Pathol, 1992. 61(6): p. 359-66.
76. Han, K., H. Zhou, and U. Pfeifer, Inhibition and restimulation by insulin of cellular autophagy in distal tubular cells of the kidney in early diabetic rats. Kidney Blood Press Res, 1997. 20(4): p. 258-63.
77. Vallon, V., et al., Knockout of Na-glucose transporter SGLT2 attenuates hyperglycemia and glomerular hyperfiltration but not kidney growth or injury in diabetes mellitus. Am J Physiol Renal Physiol, 2013. 304(2): p. F156-67.
78. Yamahara, K., et al., Obesity-mediated autophagy insufficiency exacerbates proteinuria-induced tubulointerstitial lesions. J Am Soc Nephrol, 2013. 24(11): p. 1769-81.
79. Hartleben, B., et al., Autophagy influences glomerular disease susceptibility and maintains podocyte homeostasis in aging mice. J Clin Invest, 2010. 120(4): p. 1084-96.
80. Fang, L., et al., Autophagy attenuates diabetic glomerular damage through protection of hyperglycemia-induced podocyte injury. PLoS One, 2013. 8(4): p. e60546.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for improving, increasing and/or promoting podocyte and/or kidney function and/or mitigating, reducing, inhibiting and/or delaying podocyte and/or kidney degradation and/or failure in a subject in need thereof, comprising administering to the subject an inhibitor of endoplasmic reticulum (ER) stress.

2. A method for improving, increasing and/or promoting podocyte and/or kidney function and/or mitigating, reducing, inhibiting and/or delaying podocyte and/or kidney degradation and/or failure in a subject in need thereof, comprising administering to the subject an agent that increases the production and/or level of epoxygenated fatty acids.

3. A method for improving, increasing and/or promoting podocyte and/or kidney function and/or mitigating, reducing, inhibiting and/or delaying podocyte and/or kidney degradation and/or failure in a subject in need thereof, comprising co-administering to the subject an agent that increases the production and/or level of epoxygenated fatty acids and an inhibitor of endoplasmic reticular (ER) stress.

4. The method of claim 3, wherein one or both of the agent that increases the production and/or level of epoxygenated fatty acids and the inhibitor of endoplasmic reticular stress are administered at a subtherapeutic dose.

5. The method of any one of claims 3 to 4, wherein one or both of the agent that increases the production and/or level of epoxygenated fatty acids and the inhibitor of endoplasmic reticular stress are targeted to the kidneys.

6. The method of any one of claims 3 to 5, wherein the agent that increases the production and/or level of epoxygenated fatty acids and the inhibitor of endoplasmic reticular stress are concurrently co-administered.

7. The method of any one of claims 3 to 5, wherein the agent that increases the production and/or level of epoxygenated fatty acids and the inhibitor of endoplasmic reticular stress are sequentially co-administered.

8. The method of claim 1 and any one of claims 3 to 7, wherein the inhibitor of ER stress acts as a molecular chaperone that facilitates correct protein folding and/or prevents protein aggregation and/or acts to enhance autophagy.

9. The method of claim 1 and any one of claims 3 to 8, wherein the inhibitor of ER stress modifies protein folding, regulates glucose homeostasis and/or reduces lipid overload.

10. The method of claim 1 and any one of claims 3 to 9, wherein the inhibitor of endoplasmic reticular stress performs one or more of the following:
a) prevents, reduces and/or inhibits phosphorylation of PERK (Thr980), Ire1α (Ser727), eIF2α (Ser51), p38 and/or JNK1/2;
b) prevents, reduces and/or inhibits cleavage of ATF6 and/or XBP1; and/or
c) prevents, reduces and/or inhibits mRNA expression of BiP, ATF4 and/or XBP1.

11. The method of claim 1 and any one of claims 3 to 10, wherein the inhibitor of endoplasmic reticular stress is selected from the group consisting of 4-phenyl butyric acid (“PBA”), 3-phenylpropionic acid (3-PPA), 5-phenylvaleric acid (5-PVA), 6-phenylhexanoic acid (6-PHA), butyrate, tauroursodeoxycholic acid, trehalose, deuterated water, docosahexaenoic acid (“DHA”), eicosapentaenoic acid (“EPA”), vitamin C, arabitol, mannose, glycerol, betaine, sarcosine, trimethylamine-N oxide, DMSO and mixtures thereof.

12. The method of claim 1 and any one of claims 3 to 11, wherein the inhibitor of endoplasmic reticular stress is selected from the group consisting of 4-phenyl butyric acid (4-PBA), 3-phenylpropionic acid (3-PPA), 5-phenylvaleric acid (5-PVA), 6-phenylhexanoic acid (6-PHA), esters thereof, pharmaceutically acceptable salts thereof, and mixtures thereof.

13. The method of any one of claims 2 to 12, wherein the agent that increases the production and/or level of epoxygenated fatty acids comprises one or more epoxygenated fatty acids.

14. The method of any one of claims 2 to 13, wherein the epoxygenated fatty acids are selected from the group consisting of cis-epoxyeicosantrienoic acids (“EETs”), epoxides of linoleic acid, epoxides of eicosapentaenoic acid (“EPA”), epoxides of docosahexaenoic acid (“DHA”), epoxides of the arachidonic acid (“AA”), epoxides of cis-7,10,13,16,19-docosapentaenoic acid, and mixtures thereof.

15. The method of any one of claims 2 to 14, wherein the agent that increases the production and/or level of epoxygenated fatty acids increases the production and/or levels of cis-epoxyeicosantrienoic acids (“EETs”).

16. The method of claim 15, wherein the agent that increases the production and/or level of EETs is an inhibitor of soluble epoxide hydrolase (“sEH”).

17. The method of claim 16, wherein the inhibitor of sEH comprises an inhibitory nucleic acid that specifically targets soluble epoxide hydrolase (“sEH”).

18. The method of claim 17, wherein the inhibitory nucleic acid is targeted to kidney tissue.

19. The method of any one of claims 17 to 18, wherein the inhibitory nucleic acid is targeted to podocyte cells.

20. The method of any one of claims 17 to 19, wherein the inhibitory nucleic acid is selected from the group consisting of short interfering RNA (siRNA), short hairpin RNA (shRNA), small temporal RNA (stRNA), and micro-RNA (miRNA).

21. The method of claim 16, wherein the inhibitor of sEH comprises a primary pharmacophore selected from the group consisting of a urea, a carbamate, and an amide.

22. The method of claim 21, wherein the inhibitor of sEH comprises a cyclohexyl moiety, aromatic moiety, substituted aromatic moiety or alkyl moiety attached to the pharmacophore.

23. The method of any claims 21 to 22, wherein the inhibitor of sEH comprises a cyclohexyl ether moiety attached to the pharmacophore.

24. The method of any one of claims 21 to 23, wherein the inhibitor of sEH comprises a phenyl ether or piperidine moiety attached to the pharmacophore.

25. The method of any one of claims 21 to 24, wherein the inhibitor of sEH comprises a polyether secondary pharmacophore.

26. The method of any one of claims 21 to 25, wherein the inhibitor of sEH has an IC50 of less than about 100 μM.

27. The method of any one of claims 21 to 26, wherein the inhibitor of sEH is selected from the group consisting of:
a) 3-(4-chlorophenyl)-1-(3,4-dichlorphenyl)urea or 3,4,4′-trichlorocarbanilide (TCC; compound 295);
b) 12-(3-adamantan-1-yl-ureido) dodecanoic acid (AUDA; compound 700);
c) 1-adamantanyl-3-{5-[2-(2-ethoxyethoxy)ethoxy]pentyl]}urea (AEPU; compound 950);
d) 1-(1-acetypiperidin-4-yl)-3-adamantanylurea (APAU; compound 1153);
e) trans-4-[4-(3-Adamantan-1-yl-ureido)-cyclohexyloxy]-benzoic acid (tAUCB; compound 1471);
f) cis-4-[4-(3-Adamantan-1-yl-ureido)-cyclohexyloxy]-benzoic acid (cAUCB; compound 1686);
g) 1-(1-methylsulfonyl-piperidin-4-yl)-3-(4-trifluoromethoxy-phenyl)-urea (TUPS; compound 1709);
h) trans-4-{4-[3-(4-Trifluoromethoxy-phenyl)-ureido]-cyclohexyloxy}-benzoic acid (tTUCB; compound 1728);
i) 1-trifluoromethoxyphenyl-3-(1-propionylpiperidin-4-yl) urea (TPPU; compound 1770);
j) 1-(1-ethylsulfonyl-piperidin-4-yl)-3-(4-trifluoromethoxy-phenyl)-urea (TUPSE; compound 2213);
k) 1-(1-(cyclopropanecarbonyl)piperidin-4-yl)-3-(4-(trifluoromethoxy)phenyl)urea (CPTU; compound 2214);
l) trans-N-methyl-4-[4-(3-Adamantan-1-yl-ureido)-cyclohexyloxy]-benzamide (tMAUCB; compound 2225);
m) trans-N-methyl-4-[4-((3-trifluoromethyl-4-chlorophenyl)-ureido)-cyclohexyloxy]-benzamide (tMTCUCB; compound 2226);
n) cis-N-methyl-4-{4-[3-(4-trifluoromethoxy-phenyl)-ureido]-cyclohexyloxy}-benzamide (cMTUCB; compound 2228);
o) 1-cycloheptyl-3-(3-(1,5-diphenyl-1H-pyrazol-3-yl)propyl)urea (HDP₃U; compound 2247);
p) trans-2-(4-(4-(3-(4-trifluoromethoxy-phenyl)-ureido)-cyclohexyloxy)-benzamido)-acetic acid (compound 2283);
q) N-(methylsulfonyl)-4-(trans-4-(3-(4-trifluoromethoxy-phenyl)-ureido)-cyclohexyloxy)-benzamide (compound 2728);
r) 1-(trans-4-(4-(1H-tetrazol-5-yl)-phenoxy)-cyclohexyl)-3-(4-(trifluoromethoxy)-phenyl)-urea (compound 2806);
s) 4-(trans-4-(3-(2-fluorophenyl)-ureido)-cyclohexyloxy)-benzoic acid (compound 2736);
t) 4-(4-(3-(4-(trifluoromethoxy)-phenyl)-ureido)-phenoxy)-benzoic acid (compound 2803);
u) 4-(3-fluoro-4-(3-(4-(trifluoromethoxy)-phenyl)-ureido)-phenoxy)-benzoic acid (compound 2807);
v) N-hydroxy-4-(trans-4-(3-(4-(trifluoromethoxy)-phenyl)-ureido)-cyclohexyloxy)-benzamide (compound 2761);
w) (5-methyl-2-oxo-1,3-dioxol-4-yl)methyl 4-((1r,4r)-4-(3-(4-(trifluoromethoxy)-phenyl)-ureido)-cyclohexyloxy)-benzoate (compound 2796);
x) 1-(4-oxocyclohexyl)-3-(4-(trifluoromethoxy)-phenyl)-urea (compound 2809);
y) methyl 4-(4-(3-(4-(trifluoromethoxy)-phenyl)-ureido)-cyclohexylamino)-benzoate (compound 2804);
z) 1-(4-(pyrimidin-2-yloxy)-cyclohexyl)-3-(4-(trifluoromethoxy)-phenyl)-urea (compound 2810); and
aa) 4-(trans-4-(3-(4-(difluoromethoxy)-phenyl)-ureido)-cyclohexyloxy)-benzoic acid (compound 2805).

28. The method of any one of claims 21 to 26, wherein the inhibitor of sEH is co-administered at a subtherapeutic dose.

29. The method of any one of claims 1 to 28, wherein the subject is a human.

30. The method of any one of claims 1 to 29, wherein the subject has or is suspected of having diabetes.

31. The method of any one of claims 1 to 29, wherein the subject has or is suspected of having pre-diabetes.

32. The method of any one of claims 1 to 31, wherein the subject is exhibiting one or more symptoms of renal function deficiency.

33. The method of any one of claims 1 to 32, wherein the subject is exhibiting one or more symptoms selected from the group consisting of proteinuria, renal inflammation and decline in glomerular filtration barrier (GFB).

34. The method of any one of claims 1 to 33, further comprising co-administering an inhibitor of sodium-glucose cotransporter-2 (SGLT2).

35. The method of claim 34, wherein the inhibitor of SGLT2 is selected from the group consisting of canagliflozin, dapagliflozin, empagliflozin, metformin, linagliptin, and mixtures thereof.

36. A kit for use in improving, increasing and/or promoting podocyte and/or kidney function and/or mitigating, reducing, inhibiting and/or delaying podocyte and/or kidney degradation and/or failure in a subject in need thereof, the kit comprising an agent that increases the production and/or level of epoxygenated fatty acids and an inhibitor of endoplasmic reticular stress.

37. The kit of claim 36, wherein the inhibitor of endoplasmic reticular stress is selected from the group consisting of 4-phenyl butyric acid (“PBA”), 3-phenylpropionic acid (3-PPA), 5-phenylvaleric acid (5-PVA), 6-phenylhexanoic acid (6-PHA), butyrate, tauroursodeoxycholic acid, trehalose, deuterated water, docosahexaenoic acid (“DHA”), eicosapentaenoic acid (“EPA”), vitamin C, arabitol, mannose, glycerol, betaine, sarcosine, trimethylamine-N oxide, DMSO and mixtures thereof.

38. The kit of claim 36, wherein the inhibitor of endoplasmic reticular stress is selected from the group consisting of 4-phenyl butyric acid (4-PBA), 3-phenylpropionic acid (3-PPA), 5-phenylvaleric acid (5-PVA), 6-phenylhexanoic acid (6-PHA), esters thereof, pharmaceutically acceptable salts thereof and mixtures thereof.

39. The kit of any one of claims 36 to 38, wherein the agent that increases the production and/or level of EETs is an inhibitory nucleic acid that specifically targets soluble epoxide hydrolase (“sEH”).

40. The kit of any one of claims 36 to 38, wherein the agent that increases the production and/or level of EETs is an inhibitor of soluble epoxide hydrolase (“sEH”).

41. The kit of claim 40, wherein the inhibitor of sEH comprises a primary pharmacophore selected from the group consisting of a urea, a carbamate, and an amide.

42. The kit of any one of claims 40 to 41, wherein the inhibitor of sEH comprises a cyclohexyl moiety, aromatic moiety, substituted aromatic moiety or alkyl moiety attached to the pharmacophore.

43. The kit of any one of claims 40 to 41, wherein the inhibitor of sEH comprises a cyclohexyl ether moiety attached to the pharmacophore.

44. The kit of any one of claims 40 to 41, wherein the inhibitor of sEH comprises a phenyl ether or piperidine moiety attached to the pharmacophore.

45. The kit of any one of claims 40 to 44, wherein the inhibitor of sEH comprises a polyether secondary pharmacophore.

46. The kit of any one of claims 40 to 45, wherein the inhibitor of sEH has an IC50 of less than about 100 μM.

47. The kit of any one of claims 40 to 46, wherein the inhibitor of sEH is selected from the group consisting of:
a) 3-(4-chlorophenyl)-1-(3,4-dichlorphenyl)urea or 3,4,4′-trichlorocarbanilide (TCC; compound 295);
b) 12-(3-adamantan-1-yl-ureido) dodecanoic acid (AUDA; compound 700);
c) 1-adamantanyl-3-{5-[2-(2-ethoxyethoxy)ethoxy]pentyl]}urea (AEPU; compound 950);
d) 1-(1-acetypiperidin-4-yl)-3-adamantanylurea (APAU; compound 1153);
e) trans-4-[4-(3-Adamantan-1-yl-ureido)-cyclohexyl oxy]-benzoic acid (tAUCB; compound 1471);
f) cis-4-[4(3-Adamantan-1-yl-ureido)-cyclohexyloxy]-benzoic acid (cAUCB; compound 1686);
g) 1-(1-methylsulfonyl-piperidin-4-yl)-3-(4-trifluoromethoxy-phenyl)-urea (TUPS; compound 1709);
h) trans-4-{4-[3-(4-Trifluoromethoxy-phenyl)-ureido]-cyclohexyloxy}-benzoic acid (tTUCB; compound 1728);
i) 1-trifluoromethoxyphenyl-3-(1-propionylpiperidin-4-yl) urea (TPPU; compound 1770);
j) 1-(1-ethyl sulfonyl-piperidin-4-yl)-3-(4-trifluoromethoxy-phenyl)-urea (TUPSE; compound 2213);
k) 1-(1-(cyclopropanecarbonyl)piperidin-4-yl)-3-(4-(trifluoromethoxy)phenyl)urea (CPTU; compound 2214);
l) trans-N-methyl-4-[4-(3-Adamantan-1-yl-ureido)-cyclohexyloxy]-benzamide (tMAUCB; compound 2225);
m) trans-N-methyl-4-[4-((3-trifluoromethyl-4-chlorophenyl)-ureido)-cyclohexyloxy]-benzamide (tMTCUCB; compound 2226);
n) cis-N-methyl-4-{4-[3-(4-trifluoromethoxy-phenyl)-ureido]-cyclohexyloxy}-benzamide (cMTUCB; compound 2228);
o) 1-cycloheptyl-3-(3-(1,5-diphenyl-1H-pyrazol-3-yl)propyl)urea (HDP₃U; compound 2247);
p) trans-2-(4-(4-(3-(4-trifluoromethoxy-phenyl)-ureido)-cyclohexyloxy)-benzamido)-acetic acid (compound 2283);
q) N-(methylsulfonyl)-4-(trans-4-(3-(4-trifluoromethoxy-phenyl)-ureido)-cyclohexyloxy)-benzamide (compound 2728);
r) 1-(trans-4-(4-(1H-tetrazol-5-yl)-phenoxy)-cyclohexyl)-3-(4-(trifluoromethoxy)-phenyl)-urea (compound 2806);
s) 4-(trans-4-(3-(2-fluorophenyl)-ureido)-cyclohexyloxy)-benzoic acid (compound 2736);
t) 4-(4-(3-(4-(trifluoromethoxy)-phenyl)-ureido)-phenoxy)-benzoic acid (compound 2803);
u) 4-(3-fluoro-4-(3-(4-(trifluoromethoxy)-phenyl)-ureido)-phenoxy)-benzoic acid (compound 2807);
v) N-hydroxy-4-(trans-4-(3-(4-(trifluoromethoxy)-phenyl)-ureido)-cyclohexyloxy)-benzamide (compound 2761);
w) (5-methyl-2-oxo-1,3-dioxol-4-yl)methyl 4-((1r,4r)-4-(3-(4-(trifluoromethoxy)-phenyl)-ureido)-cyclohexyloxy)-benzoate (compound 2796);
x) 1-(4-oxocyclohexyl)-3-(4-(trifluoromethoxy)-phenyl)-urea (compound 2809);
y) methyl 4-(4-(3-(4-(trifluoromethoxy)-phenyl)-ureido)-cyclohexylamino)-benzoate (compound 2804);
z) 1-(4-(pyrimidin-2-yloxy)-cyclohexyl)-3-(4-(trifluoromethoxy)-phenyl)-urea (compound 2810); and
aa) 4-(trans-4-(3-(4-(difluoromethoxy)-phenyl)-ureido)-cyclohexyloxy)-benzoic acid (compound 2805).

48. The kit of any one of claims 36 to 46, further comprising an inhibitor of sodium-glucose cotransporter-2 (SGLT2).

49. The kit of claim 48, wherein the inhibitor of SGLT2 is selected from the group consisting of canagliflozin, dapagliflozin, empagliflozin, metformin, linagliptin, and mixtures thereof.

50. The kit of any one of claims 36 to 49, wherein the agent that increases the production and/or level of epoxygenated fatty acids and the inhibitor of endoplasmic reticular stress are provided in a mixture.

51. The kit of any one of claims 36 to 49, wherein the agent that increases the production and/or level of epoxygenated fatty acids and the inhibitor of endoplasmic reticular stress are provided in separate containers.

52. The kit of any one of claims 36 to 51, wherein one or both of the agent that increases the production and/or level of epoxygenated fatty acids and the inhibitor of endoplasmic reticular stress are targeted to the kidneys.

53. A composition comprising an agent that increases the production and/or level of epoxygenated fatty acids and an inhibitor of endoplasmic reticular (ER) stress.

54. The composition of claim 53, wherein the inhibitor of endoplasmic reticular stress is selected from the group consisting of 4-phenyl butyric acid (“PBA”), 3-phenylpropionic acid (3-PPA), 5-phenylvaleric acid (5-PVA), 6-phenylhexanoic acid (6-PHA), butyrate, tauroursodeoxycholic acid, trehalose, deuterated water, docosahexaenoic acid (“DHA”), eicosapentaenoic acid (“EPA”), vitamin C, arabitol, mannose, glycerol, betaine, sarcosine, trimethylamine-N oxide, DMSO and mixtures thereof.

55. The composition of claim 53, wherein the inhibitor of endoplasmic reticular stress is selected from the group consisting of 4-phenyl butyric acid (4-PBA), 3-phenylpropionic acid (3-PPA), 5-phenylvaleric acid (5-PVA), 6-phenylhexanoic acid (6-PHA), esters thereof, pharmaceutically acceptable salts thereof and mixtures thereof.

56. The composition of any one of claims 53 to 55, wherein the agent that increases the production and/or level of EETs is an inhibitory nucleic acid that specifically targets soluble epoxide hydrolase (“sEH”).

57. The composition of any one of claims 53 to 56, wherein the agent that increases the production and/or level of EETs is an inhibitor of soluble epoxide hydrolase (“sEH”).

58. The composition of claim 57, wherein the inhibitor of sEH comprises a primary pharmacophore selected from the group consisting of a urea, a carbamate, and an amide.

59. The composition of any one of claims 57 to 58, wherein the inhibitor of sEH comprises a cyclohexyl moiety, aromatic moiety, substituted aromatic moiety or alkyl moiety attached to the pharmacophore.

60. The composition of any one of claims 57 to 59, wherein the inhibitor of sEH comprises a cyclohexyl ether moiety attached to the pharmacophore.

61. The composition of any one of claims 57 to 60, wherein the inhibitor of sEH comprises a phenyl ether or piperidine moiety attached to the pharmacophore.

62. The composition of any one of claims 57 to 61, wherein the inhibitor of sEH comprises a polyether secondary pharmacophore.

63. The composition of any one of claims 57 to 62, wherein the inhibitor of sEH has an IC50 of less than about 100 μM.

64. The composition of any one of claims 57 to 63, wherein the inhibitor of sEH is selected from the group consisting of:
a) 3-(4-chlorophenyl)-1-(3,4-dichlorphenyl)urea or 3,4,4′-trichlorocarbanilide (TCC; compound 295);
b) 12-(3-adamantan-1-yl-ureido) dodecanoic acid (AUDA; compound 700);
c) 1-adamantanyl-3-{5-[2-(2-ethoxyethoxy)ethoxy]pentyl]}urea (AEPU; compound 950);
d) 1-(1-acetypiperidin-4-yl)-3-adamantanylurea (APAU; compound 1153);
e) trans-4-[4-(3-Adamantan-1-yl-ureido)-cyclohexyloxy]-benzoic acid (tAUCB; compound 1471);
f) cis-4-[4-(3-Adamantan-1-yl-ureido)-cyclohexyloxy]-benzoic acid (cAUCB; compound 1686);
g) 1-(1-methylsulfonyl-piperidin-4-yl)-3-(4-trifluoromethoxy-phenyl)-urea (TUPS; compound 1709);
h) trans-4-{4-[3-(4-Trifluoromethoxy-phenyl)-ureido]-cyclohexyloxy}-benzoic acid (tTUCB; compound 1728);
i) 1-trifluoromethoxyphenyl-3-(1-propionylpiperidin-4-yl) urea (TPPU; compound 1770);
j) 1-(1-ethylsulfonyl-piperidin-4-yl)-3-(4-trifluoromethoxy-phenyl)-urea (TUPSE; compound 2213);
k) 1-(1-(cyclopropanecarbonyl)piperidin-4-yl)-3-(4-(trifluoromethoxy)phenyl)urea (CPTU; compound 2214);
l) trans-N-methyl-4-[4-(3-Adamantan-1-yl-ureido)-cyclohexyloxy]-benzamide (tMAUCB; compound 2225);
m) trans-N-methyl-4-[4-((3-trifluoromethyl-4-chlorophenyl)-ureido)-cyclohexyloxy]-benzamide (tMTCUCB; compound 2226);
n) cis-N-methyl-4-{4-[3-(4-trifluoromethoxy-phenyl)-ureido]-cyclohexyloxy}-benzamide (cMTUCB; compound 2228);
o) 1-cycloheptyl-3-(3-(1,5-diphenyl-1H-pyrazol-3-yl)propyl)urea (HDP₃U; compound 2247);
p) trans-2-(4-(4-(3-(4-trifluoromethoxy-phenyl)-ureido)-cyclohexyloxy)-benzamido)-acetic acid (compound 2283);
q) N-(methylsulfonyl)-4-(trans-4-(3-(4-trifluoromethoxy-phenyl)-ureido)-cyclohexyloxy)-benzamide (compound 2728);
r) 1-(trans-4-(4-(1H-tetrazol-5-yl)-phenoxy)-cyclohexyl)-3-(4-(trifluoromethoxy)-phenyl)-urea (compound 2806);
s) 4-(trans-4-(3-(2-fluorophenyl)-ureido)-cyclohexyloxy)-benzoic acid (compound 2736);
t) 4-(4-(3-(4-(trifluoromethoxy)-phenyl)-ureido)-phenoxy)-benzoic acid (compound 2803);
u) 4-(3-fluoro-4-(3-(4-(trifluoromethoxy)-phenyl)-ureido)-phenoxy)-benzoic acid (compound 2807);
v) N-hydroxy-4-(trans-4-(3-(4-(trifluoromethoxy)-phenyl)-ureido)-cyclohexyloxy)-benzamide (compound 2761);
w) (5-methyl-2-oxo-1,3-dioxol-4-yl)methyl 4-((1r,4r)-4-(3-(4-(trifluoromethoxy)-phenyl)-ureido)-cyclohexyloxy)-benzoate (compound 2796);
x) 1-(4-oxocyclohexyl)-3-(4-(trifluoromethoxy)-phenyl)-urea (compound 2809);
y) methyl 4-(4-(3-(4-(trifluoromethoxy)-phenyl)-ureido)-cyclohexylamino)-benzoate (compound 2804);
z) 1-(4-(pyrimidin-2-yloxy)-cyclohexyl)-3-(4-(trifluoromethoxy)-phenyl)-urea (compound 2810); and
aa) 4-(trans-4-(3-(4-(difluoromethoxy)-phenyl)-ureido)-cyclohexyloxy)-benzoic acid (compound 2805).

65. The composition of any one of claims 57 to 64, wherein one or both of the agent that increases the production and/or level of epoxygenated fatty acids and the inhibitor of endoplasmic reticular stress are targeted to the kidneys.


SEQUENCE LISTING (38 sequences)

SEQ ID NO: 1 - protein, 554 aa, Homo sapiens
MTLRGAVFDLDGVLALPAVFGVLGRTEEALALPRGLLNDAFQKGGPEGATTRLMKGEITLSQWIPLMEENCRKCSETAKVCLPKNFSIKEIFDKAISARKINRPMLQAALMLRKKGFTTAILTNTWLDDRAERDGLAQLMCELKMHFDFLIESCQVGMVKPEPQIYKFLLDTLKASPSEVVFLDDIGANLKPARDLGMVTILVQDTDTALKELEKVTGIQLLNTPAPLPTSCNPSDMSHGYVTVKPRVRLHFVELGWPAVCLCHGFPESWYSWRYQIPALAQAGYRVLAMDMKGYGESSAPPEIEEYCMEVLCKEMVTFLDKLGLSQAVFIGHDWGGMLVWYMALFYPERVRAVASLNTPFIPANPNMSPLESIKANPVFDYQLYFQEPGVAEAELEQNLSRTFKSLFRASDESVLSMHKVCEAGGLFVNSPEEPSLSRMVTEEEIQFYVQQFKKSGFRGPLNWYRNMERNWKWACKSLGRKILIPALMVTAEKDFVLVPQMSQHMEDWIPHLKRGHIEDCGHWTQMDKPTEVNQILIKWLDSDARNPPVVSKM

SEQ ID NO: 2 - DNA, 2100 bp, Homo sapiens
ggcacgagctctctctctctctctctctctctctcgccgccatgacgctgcgcggcgccgtcttcgaccttgacggggtgctggcgctgccagcggtgttcggcgtcctcggccgcacggaggaggccctggcgctgcccagaggacttctgaatgatgctttccagaaagggggaccagagggtgccactacccggcttatgaaaggagagatcacactttcccagtggataccactcatggaagaaaactgcaggaagtgctccgagaccgctaaagtctgcctccccaagaatttctccataaaagaaatctttgacaaggcgatttcagccagaaagatcaaccgccccatgctccaggcagctctcatgctcaggaagaaaggattcactactgccatcctcaccaacacctggctggacgaccgtgctgagagagatggcctggcccagctgatgtgtgagctgaagatgcactttgacttcctgatagagtcgtgtcaggtgggaatggtcaaacctgaacctcagatctacaagtttctgctggacaccctgaaggccagccccagtgaggtcgtttttttggatgacatcggggctaatctgaagccagcccgtgacttgggaatggtcaccatcctggtccaggacactgacacggccctgaaagaactggagaaagtgaccggaatccagcttctcaataccccggcccctctgccgacctcttgcaatccaagtgacatgagccatgggtacgtgacagtaaagcccagggtccgtctgcattttgtggagctgggctggcctgctgtgtgcctctgccatggatttcccgagagttggtattcttggaggtaccagatccctgctctggcccaggcaggttaccgggtcctagctatggacatgaaaggctatggagagtcatctgctcctcccgaaatagaagaatattgcatggaagtgttatgtaaggagatggtaaccttcctggataaactgggcctctctcaagcagtgttcattggccatgactggggtggcatgctggtgtggtacatggctctcttctaccccgagagagtgagggcggtggccagtttgaatactcccttcataccagcaaatcccaacatgtcccctttggagagtatcaaagccaacccagtatttgattaccagctctacttccaagaaccaggagtggctgaggctgaactggaacagaacctgagtcggactttcaaaagcctcttcagagcaagcgatgagagtgttttatccatgcataaagtctgtgaagcgggaggactttttgtaaatagcccagaagagcccagcctcagcaggatggtcactgaggaggaaatccagttctatgtgcagcagttcaagaagtctggtttcagaggtcctctaaactggtaccgaaacatggaaaggaactggaagtgggcttgcaaaagcttgggacggaagatcctgattccggccctgatggtcacggcggagaaggacttcgtgctcgttcctcagatgtcccagcacatggaggactggattccccacctgaaaaggggacacattgaggactgtgggcactggacacagatggacaagccaaccgaggtgaatcagatcctcattaagtggctggattctgatgcccggaacccaccggtggtctcaaagatgtagaacgcagcgtagtgcccacgctcagcaggtgtgccatccttccacctgctggggcaccattcttagtatacagaggtggccttacacacatcttgcatggatggcagcattgttctgaaggggtttgcagaaaaaaaagattttctttacataaagtgaatcaaatttgacattattttagatcccagagaaatcaggtgtgattagttctccaggcatgaatgcatcgtccctttatctgtaagaacccttagtgtcctgtagggggacagaatggggtggccaggtggtgatttctctttgaccaatgcatagtttggcagaaaaatcagccgttcatttagaagaatcttagcagagattgggatgccttactcaataaagctaagatgac

SEQ ID NO: 3 - DNA, 23 bp, Artificial Sequence
  Description of Artificial Sequence Synthetic oligonucleotide
cagtgttcattggccatgactgg

SEQ ID NO: 4 - DNA, 21 bp, Artificial Sequence
  Description of Artificial Sequence Synthetic oligonucleotide
  Description of Combined DNA/RNA Molecule Synthetic oligonucleotide
guguucauuggccaugacutt

SEQ ID NO: 5 - DNA, 21 bp, Artificial Sequence
  Description of Artificial Sequence Synthetic oligonucleotide
  Description of Combined DNA/RNA Molecule Synthetic oligonucleotide
agucauggccaaugaacactt

SEQ ID NO: 6 - DNA, 23 bp, Artificial Sequence
  Description of Artificial Sequence Synthetic oligonucleotide
gaaaggctatggagagtcatctg

SEQ ID NO: 7 - DNA, 21 bp, Artificial Sequence
  Description of Artificial Sequence Synthetic oligonucleotide
  Description of Combined DNA/RNA Molecule Synthetic oligonucleotide
aaggcuauggagagucauctt

SEQ ID NO: 8 - DNA, 21 bp, Artificial Sequence
  Description of Artificial Sequence Synthetic oligonucleotide
  Description of Combined DNA/RNA Molecule Synthetic oligonucleotide
gaugacucuccauagccuutt

SEQ ID NO: 9 - DNA, 23 bp, Artificial Sequence
  Description of Artificial Sequence Synthetic oligonucleotide
aaaggctatggagagtcatctgc

SEQ ID NO: 10 - DNA, 21 bp, Artificial Sequence
  Description of Artificial Sequence Synthetic oligonucleotide
  Description of Combined DNA/RNA Molecule Synthetic oligonucleotide
aggcuauggagagucaucutt

SEQ ID NO: 11 - DNA, 21 bp, Artificial Sequence
  Description of Artificial Sequence Synthetic oligonucleotide
  Description of Combined DNA/RNA Molecule Synthetic oligonucleotide
agaugacucuccauagccutt

SEQ ID NO: 12 - DNA, 23 bp, Artificial Sequence
  Description of Artificial Sequence Synthetic oligonucleotide
caagcagtgttcattggccatga

SEQ ID NO: 13 - DNA, 21 bp, Artificial Sequence
  Description of Artificial Sequence Synthetic oligonucleotide
  Description of Combined DNA/RNA Molecule Synthetic oligonucleotide
agcaguguucauuggccautt

SEQ ID NO: 14 - DNA, 21 bp, Artificial Sequence
  Description of Artificial Sequence Synthetic oligonucleotide
  Description of Combined DNA/RNA Molecule Synthetic oligonucleotide
auggccaaugaacacugcutt

SEQ ID NO: 15 - DNA, 23 bp, Artificial Sequence
  Description of Artificial Sequence Synthetic oligonucleotide
cagcacatggaggactggattcc

SEQ ID NO: 16 - DNA, 21 bp, Artificial Sequence
  Description of Artificial Sequence Synthetic oligonucleotide
  Description of Combined DNA/RNA Molecule Synthetic oligonucleotide
gcacauggaggacuggauutt

SEQ ID NO: 17 - DNA, 21 bp, Artificial Sequence
  Description of Artificial Sequence Synthetic oligonucleotide
  Description of Combined DNA/RNA Molecule Synthetic oligonucleotide
aauccaguccuccaugugctt

SEQ ID NO: 18 - DNA, 9 bp, Artificial Sequence
  Description of Artificial Sequence Synthetic oligonucleotide
ttcaagaga

SEQ ID NO: 19 - DNA, 23 bp, Artificial Sequence
  Description of Artificial Sequence Synthetic oligonucleotide
cagtgttcattggccatgactgg

SEQ ID NO: 20 - DNA, 59 bp, Artificial Sequence
  Description of Artificial Sequence Synthetic oligonucleotide
gatccccgtgttcattggccatgactttcaagagaagtcatggccaatgaacacttttt

SEQ ID NO: 21 - DNA, 59 bp, Artificial Sequence
  Description of Artificial Sequence Synthetic oligonucleotide
agctaaaaagtgttcattggccatgacttctcttgaaagtcatggccaatgaacacggg

SEQ ID NO: 22 - DNA, 23 bp, Artificial Sequence
  Description of Artificial Sequence Synthetic oligonucleotide
gaaaggctatggagagtcatctg

SEQ ID NO: 23 - DNA, 59 bp, Artificial Sequence
  Description of Artificial Sequence Synthetic oligonucleotide
gatccccaaggctatggagagtcatcttcaagagagatgactctccatagccttttttt

SEQ ID NO: 24 - DNA, 59 bp, Artificial Sequence
  Description of Artificial Sequence Synthetic oligonucleotide
agctaaaaaaaggctatggagagtcatctctcttgaagatgactctccatagccttggg

SEQ ID NO: 25 - DNA, 23 bp, Artificial Sequence
  Description of Artificial Sequence Synthetic oligonucleotide
aaaggctatggagagtcatctgc

SEQ ID NO: 26 - DNA, 59 bp, Artificial Sequence
  Description of Artificial Sequence Synthetic oligonucleotide
gatccccaggctatggagagtcatctttcaagagaagatgactctccatagcctttttt

SEQ ID NO: 27 - DNA, 59 bp, Artificial Sequence
  Description of Artificial Sequence Synthetic oligonucleotide
agctaaaaaaggctatggagagtcatcatctcttgaaagatgactctccatagcctggg

SEQ ID NO: 28 - DNA, 23 bp, Artificial Sequence
  Description of Artificial Sequence Synthetic oligonucleotide
caagcagtgttcattggccatga

SEQ ID NO: 29 - DNA, 59 bp, Artificial Sequence
  Description of Artificial Sequence Synthetic oligonucleotide
gatccccagcagtgttcattggccatttcaagagaatggccaatgaacactgctttttt

SEQ ID NO: 30 - DNA, 59 bp, Artificial Sequence
  Description of Artificial Sequence Synthetic oligonucleotide
agctaaaaaagcagtgttcattggccattctcttgaaatggccaatgaacactgctggg

SEQ ID NO: 31 - DNA, 23 bp, Artificial Sequence
  Description of Artificial Sequence Synthetic oligonucleotide
cagcacatggaggactggattcc

SEQ ID NO: 32 - DNA, 59 bp, Artificial Sequence
  Description of Artificial Sequence Synthetic oligonucleotide
gatccccgcacatggaggactggattttcaagagaaatccagtcctccatgtgcttttt

SEQ ID NO: 33 - DNA, 59 bp, Artificial Sequence
  Description of Artificial Sequence Synthetic oligonucleotide
agctaaaaagcacatggaggactggatttctcttgaaaatccagtcctccatgtgcggg

SEQ ID NO: 34 - RNA, 20 nt, Artificial Sequence
  Description of Artificial Sequence Synthetic oligonucleotide
uguccagugcccacaguccu

SEQ ID NO: 35 - RNA, 20 nt, Artificial Sequence
  Description of Artificial Sequence Synthetic oligonucleotide
uucccaccugacacgacucu

SEQ ID NO: 36 - RNA, 20 nt, Artificial Sequence
  Description of Artificial Sequence Synthetic oligonucleotide
guucagccucagccacuccu

SEQ ID NO: 37 - RNA, 19 nt, Artificial Sequence
  Description of Artificial Sequence Synthetic oligonucleotide
aguccucccgcuucacaga

SEQ ID NO: 38 - RNA, 21 nt, Artificial Sequence
  Description of Artificial Sequence Synthetic oligonucleotide
gcccacuuccaguuccuuucc